(19) United States
(12) Patent Application Publication
(10) Pub. No.: US 2005/0136480 A1
(43) Pub. Date: Jun. 23, 2005

(54) Computer based versatile method for identifying protein coding DNA sequences useful as drug targets

(76) Inventors: Samir Kumar Brahmachari, Delhi (IN); Debasis Dash, Delhi (IN); Ramakant Sharma, Delhi (IN); Jitendra Kumar Maheshwari, Delhi (IN)

Correspondence Address: ARENT FOX PLLC, 1050 CONNECTICUT AVENUE, N.W., SUITE 400, WASHINGTON, DC, 20036, US

(21) Appl. No.: 10/755,415

(22) Filed: Jan. 13, 2004

Related U.S. Application Data
(63) Continuation-in-part of application No. 10/727,989, filed on Dec. 5, 2003.

(51) Int. Cl. G01N 33/53; G06F 19/00; G01N 33/48; G01N 33/50

(52) U.S. Cl. 435/7.1; 702/19

(57) ABSTRACT

The present invention relates to a versatile method of identifying protein coding DNA sequences (genes) useful as drug targets in a genome using specially developed software GeneDecipher, said method comprising steps of generating peptide libraries from the known genomes with peptide of length ‘N’ computationally arranged in an alphabetical order, artificially translating the test genome to obtain a polypeptide corresponding to each reading frame, converting each polypeptide sequence into an alphanumeric sequence one corresponding to each reading frame on the basis of overlappings with the peptide libraries, training Artificial Neural Network (ANN) with sigmoidal learning function to the alphanumeric sequence, deciphering the protein coding regions in the test genome, thus, identifying longer streches of peptides mapping to large number of known genes and their corresponding proteins and lastly, a method of the management of the diseases caused by the pathogenic organisms comprising a step of evaluation of the proposed drug candidate by inhibiting the functioning of one or more proteins identified by the steps of the invention.

### FIELD OF THE PRESENT INVENTION

This invention relates to a versatile method for identifying protein coding DNA sequences useful as drug targets. More particularly this invention relates to a method for identification of novel genes in genome sequence data of various organisms, useful as potential drug targets. This invention further provides a method for assignment of function to hypothetical Open Reading Frames (proteins) of unknown function through exact amino acid sequence identity signature.

Emergence of high throughput sequencing technologies has necessitated identification of novel protein coding DNA sequences (genes) in newly sequenced genomes. The invention provides a novel method of converting DNA sequence to alphanumeric sequence by the use of peptide library. The invention also provides a method for use of artificial neural network (feed forward back propagation topology) with one input layer, one hidden layer with 30 neurons and one output layer for identification protein coding DNA sequences. The invention further provides a method for training of neural networks using sigmoid as a learning function with five parameters namely total score, mean, fraction of zeroes, maximum continuous non-zero stretch and variance for identification of protein coding DNA sequence.

### BACKGROUND AND PRIOR ART REFERENCES OF THE PRESENT INVENTION

The most reliable way to identify a protein coding DNA sequence (gene) in a newly sequenced genome is to find a close homolog from other organisms (BLAST (Altschul, S. F et al., 1990) and FASTA (Pearson, W. R., 1995)). Four nucleotides in a DNA sequence are not randomly distributed. The statistical distribution of nucleotides within a coding region is significantly different from the non-coding (Bird, A., 1987). Methods based on Hidden Markov Models (HMM) have used these statistical properties most efficiently (Salzberg, S. L et al., 1998; Delcher, A. L et al., 1999; Lukashin, A. V. and Borodovsky, M., 1998) and are able to predict ˜97-98% of all the genes in a genome when compared with published annotations (Delcher, A. L et al., 1999). Using HMM, various algorithms like GeneMark, Glimmer etc. have been developed to predict genes in prokaryotes. Glimmer 2.0 is the most successful method among all existing methods (Delcher, A. L et al., 1999). However, Glimmer also predicts 7-20% additional genes (false positives).

Each gene prediction method has its own strengths and weaknesses (Mathe, C. et al., 2002). Since the prediction is usually dependent on the training set, shortcomings arise because statistics for a coding region vary across various genomes. Also, these methods are unable to efficiently predict genes small in length (<100 amino acids), because it's very difficult to detect these genes by similarity searches or by statistical analysis. The problem becomes more severe in case of horizontal gene transfer (Kehoe, M. A et al., 1996). In this case statistical distribution of the nucleotide sequence of these genes differs within a genome itself.

The said method of the invention is based upon the observation that the difference between total number of theoretically possible peptides of a given length and that which are actually observed in nature, increases drastically as this length of peptide increases. For example, only about 2% of the theoretically possible heptapeptides are observed in a pool of 56 completely sequenced prokaryotic genomes. At octapeptide level this number reduces to even less than 0.1%. Moreover, it is interesting to note that most of these peptides selected by nature are found only in the coding regions and very rarely in theoretically translated non-coding regions. This observation has prompted us to exploit this exclusivity of natural selection of peptides that are present in protein coding sequences to differentiate between coding and non-coding regions.

In principle, using longer peptides to score a query ORF is always preferable to using shorter ones (Salzberg, S. L. et al., 1998), but only if sufficient data is available to estimate statistical parameters required to train the prediction algorithm. In case we use peptides of length 8 or more amino acids, it is difficult to get sufficient data to estimate the training parameters. This is because likelihood of an octapeptide being shared between two polypeptides is less than that of a heptapeptide. So we consider the length of 7 amino acids as optimum for scoring of an ORF.

The novelty of the said method is that it works on the basis of protein coding sequences at amino acid, not at nucleotide sequence level. It is noteworthy that the method does not need an organism specific training set, which is an obvious advantage over other methods. Unlike other methods, GeneDecipher does not employ any landmarks like ribosome binding sites, promoter sequences, transcription start sites or codon usage biases to predict the coding genes and their start locations. In addition, this method overcomes the difficulties of gene prediction for smaller genomes (Chen, L et. al., 2003) like SARS-CoV. Other than gene prediction, this method can also be utilized for similarity searches for polypeptides, putative functional assignment to proteins (based on presence of the oligo-peptide motifs), and in phylogenetic domain analysis, indicating the generic-ness and versatility of the method.

Current computational methods like GeneMark.hmm (Lukashin and Borodovsky, 1998), Glimmer (Salzberg et al., 1998), etc. face difficulty in analyzing the small genomes such as of SARS. Methods based on Hidden Markov Models (HMM) require thousands of parameters for training. This makes these methods less suitable for analyzing smaller genomes. The problem compounds in the case of SARS-CoV genomes, which are about 30 kb length. Even the method most suitable for viral gene prediction till date ZCURVE_CoV (Chen et al., 2003) needs 33 parameters for training. GeneDecipher needs only 5 parameters and can analyze smaller genomes too. The applicants have trained the Artificial Neural Network on ecoli-k12 genome coding and non-coding regions (ORFs not reported as a gene). To predict protein coding genes using GeneDecipher on viral genomes no additional training is required. This is an obvious advantage of this method over other methods.

### OBJECTS OF THE PRESENT INVENTION

The main object of the present invention is to provide a computer based method for predicting protein coding DNA sequences (genes) useful as drug targets.

Another main object of the present invention is to develop a versatile method of identifying genes using oligopeptides that are found to occur in the ORFs of other genomes using software GeneDecipher.

Still another object of the present invention is to develop a method applicable in the management of the diseases caused by the pathogenic organisms.

Still another object of the present invention is to develop a computer based system for performing the aforementioned methods.

Yet another object of the present invention is to develop a method useful for identification of novel protein coding DNA sequences useful as potential drug targets and can serve as drug screen for broad spectrum antibacterial as well as for specific diagnosis of infection. Still another object of the present invention is to identify strain specific or organism specific protein coding genes.

Yet another object of the method of invention is to identify protein coding DNA sequences (exons) in eukaryotic organisms.

Another object of the present invention is to assignment of function to hypothetical Open Reading Frames (proteins) of unknown function through exact amino acid sequence identity signature.

### SUMMARY OF THE PRESENT INVENTION

The present invention relates to a versatile method of identifying genes using oligopeptides that are found to occur in the ORFs of other genomes and is also suitable for analyzing small genomes using software GeneDecipher, said method comprising steps of generating peptide libraries from the known genomes with peptide of length ‘N’ computationally arranged in an alphabetical order, artificially translating the test genome to obtain a polypeptide in each reading frame, converting each polypeptide sequence into an alphanumeric sequence with one corresponding to each reading frame on the basis of overlappings with the peptide libraries, training Artificial Neural Network (ANN) with sigmoidal learning function to the alphanumeric sequence, deciphering the protein coding regions in the test genome, thus, identifying longer streches of peptides mapping to large number of known genes and their corresponding proteins and lastly, a method of the management of the diseases caused by the pathogenic organisms comprising a step of evaluation of the proposed drug candidate by inhibiting the functioning of one or more proteins identified by the steps of the invention.

### DETAILED DESCRIPTION OF THE PRESENT INVENTION

Accordingly, the present invention relates to a versatile method of identifying protein coding DNA sequences (genes) useful as drug targets in a genome using specially developed software GeneDecipher, said method comprising steps of generating peptide libraries from the known genomes with peptide of length ‘N’ computationally arranged in an alphabetical order, artificially translating the test genome to obtain a polypeptide corresponding to each reading frame, converting each polypeptide sequence into an alphanumeric sequence one corresponding to each reading frame on the basis of overlappings with the peptide libraries, training Artificial Neural Network (ANN) with sigmoidal learning function to the alphanumeric sequence, deciphering the protein coding regions in the test genome, thus, identifying longer streches of peptides mapping to large number of known genes and their corresponding proteins and lastly, a method of the management of the diseases caused by the pathogenic organisms comprising a step of evaluation of the proposed drug candidate by inhibiting the functioning of one or more proteins identified by the steps of the invention.

In an embodiment of the present invention, wherein a computer based versatile method for identifying protein coding DNA sequences useful as drug targets said method comprising steps of:
generating peptide libraries from the known genomes with oligopeptide of length ‘N’ computationally arranged in an alphabetical order,
artificially translating the test genome to obtain a polypeptide in each reading frame,
converting each polypeptide sequence into an alphanumeric sequence with one corresponding to each reading frame on the basis of occurrence of these oligopeptides in the peptide libraries,
training Artificial Neural Network (ANN) with sigmoidal learning function to the alphanumeric sequences corresponding to known protein coding DNA sequences and known non-coding regions,
deciphering the protein coding regions in the test genome, and
identifying longer stretches of peptides mapped to large number of known genes serving as functional signatures.

In another embodiment of the present invention, wherein the artificial neural network has one or more input layer, one or more hidden layer with varying number of neurons, and one or more output layer.

In yet another embodiment of the present invention, wherein the number of neurons in the hidden layer is preferably 30.

In still another embodiment of the present invention, wherein the value of the ‘N’ is 4 or more.

In still another embodiment of the present invention, wherein the sigmoidal learning function has five parameters comprising total score, mean, fraction of zeroes, maximum continuous non-zero stretch, and variance.

In still another embodiment of the present invention, wherein the method of identifying genes using oligopeptides that are found to occur in the ORFs of other genomes but not limited to genomes such as H. influenzae, M. genitalium, E. coli, B. subtilis, A. fulgidis, M. tuberculosis, T. pallidum, T. maritima, Synecho cystis, H. pylori, and SARS-CoV.

In still another embodiment of the present invention, wherein a method claimed in claim 1, wherein the peptide library data may be taken from any organism but not specifically limited to those used in the invention.

In still another embodiment of the present invention, wherein a set of genes of SEQ ID Nos. 1 to 44 of H. influenzae, identified by using aforementioned method.

In still another embodiment of the present invention, wherein a set of proteins of SEQ ID Nos. 170 to 213 corresponding to genes of SEQ ID Nos 1 to 44 of H. influenzae, identified by using aforementioned method.

In still another embodiment of the present invention, wherein a set of genes of SEQ ID Nos. 45 to 60 of H. pylori, identified by using aforementioned method.

In still another embodiment of the present invention, wherein a set of proteins of SEQ ID Nos. 214 to 229 corresponding to genes of SEQ ID Nos 45 to 60 of H. pylori identified by using aforementioned method.

In still another embodiment of the present invention, wherein a set of genes of SEQ ID Nos. 61 to 165 of M. tuberculosis, identified by using aforementioned method.

In still another embodiment of the present invention, wherein a set of proteins of SEQ ID Nos. 230 to 334 corresponding to genes of SEQ ID Nos 61 to 165 of M. Tuberculosis, identified by using aforementioned method.

In still another embodiment of the present invention, wherein a set of genes of SEQ ID Nos. 166 to 169 of SARS-corona virus identified by using aforementioned method.

In still another embodiment of the present invention, wherein a set of proteins of SEQ ID Nos. 335 to 338 corresponding to genes of SEQ ID Nos 166 to 169 of SARS-corona virus, identified by using aforementioned method.

In still another embodiment of the present invention, wherein use of proteins of SEQ ID Nos. 170 to 338 corresponding to the genes of SEQ ID Nos. 1 to 169, as the drug target for the managing disease conditions caused by the pathogenic organisms in a subject in need thereof.

In still another embodiment of the present invention, wherein the pathogenic organisms are selected from a group comprising SARS-corona virus, H. influenzae, M. tuberculosis, and H. pylori.

In still another embodiment of the present invention, wherein the subject is an animal.

In still another embodiment of the present invention, wherein the subject is a human.

In still another embodiment of the present invention, wherein the use is extended to eukaryotes and multicellular organisms.

Emergence of high throughput sequencing technologies has necessitated identification of novel protein coding DNA sequences (genes) in newly sequenced genomes. The invention provides a novel method of converting DNA sequence to alphanumeric sequence by the use of peptide library. The invention also provides a method for use of artificial neural network (feed forward back propagation topology) with one input layer, one hidden layer with 30 neurons and one output layer for identification protein coding DNA sequences. The invention further provides a method for training of neural networks using sigmoid as a learning function with five parameters namely total score, mean, fraction of zeroes, maximum continuous non-zero stretch and variance for identification of protein coding DNA sequence.

The applicants have invented a novel computer based method to identify protein coding DNA sequences by comparing with peptide library containing millions of peptides obtained from protein sequences of many organisms that has withstood natural selection. The method describes a generic and versatile new approach for gene identification. The computational method determines gene candidates among all possible Open Reading Frames (ORF) of a given DNA sequence through the use of a peptide library and an artificial neural network. The peptide library consists of all possible overlapping heptapeptides derived from proteins of completely sequenced 56 or more prokaryotic genomes. A given query ORF qualifies as a gene based upon the abundance and distribution pattern of library heptapeptides (heptapeptides present in library) along the ORF. Performance of the method is characterized by simultaneous high values of sensitivity and specificity. An analysis of 10 completely sequenced prokaryotic genomes is provided to demonstrate the capabilities of the method of the invention.

The present method also allows prediction of alternate target against a specific peptide motif of a pathogenic organism or any host protein target responsible for a disease process. The method could be extended with different peptide lengths to obtain larger number of protein coding genes and also for eukaryotes and multicellular organisms.

The invention relates to a novel method of converting DNA sequence to alphanumeric sequence by the use of peptide library and the invention also provides a method for use of artificial neural network (feed forward back propagation topology) with one input layer, one hidden layer with 30 neurons and one output layer for identification protein coding DNA sequences. The invention further relates to a method for training of neural networks using sigmoid as a learning function with five parameters namely total score, mean, fraction of zeroes, maximum continuous non-zero stretch and variance for identification of protein coding DNA sequence and the present method is useful for identification of new protein coding regions which can serve as drug screen for broad-spectrum antibacterials as well as for specific diagnosis of infections, and in addition, for assignment of function to newly identified proteins of yet unknown functions. The method allows identification of species or strain specific protein coding genes. This method also can be extended to any protein coding sequence identification even in eukaryotic genomes.

Accordingly, present invention discloses a computer based versatile method for identifying protein coding DNA sequences useful as drug targets, said method comprising steps of:
a. generating peptide libraries from the known genomes with oligopeptide of length ‘N’ computationally arranged in an alphabetical order,
b. artificially translating the test genome to obtain a polypeptide in each reading frame,
c. converting each polypeptide sequence into an alphanumeric sequence with one corresponding to each reading frame on the basis of occurrence of these oligopeptides in the peptide libraries,
d. training Artificial Neural Network (ANN) with sigmoidal learning function to the alphanumeric sequences corresponding to known protein coding DNA sequences and known non-coding regions,
e. deciphering the protein coding regions in the test genome, and
f. identifying longer stretches of peptides (evolutionary conserved oligopeptides) mapped to large number of known genes serving as functional signatures.

In yet another embodiment of the present invention the ANN has one or more input layer, one or more hidden layer with varying number of neurons, and one or more output layer.

In still another embodiment of the present invention the number of neurons in the hidden layer is preferably 30.

In yet another embodiment of the present invention the value of the ‘N’ is 4 or more.

In yet another embodiment of the present invention the sigmoidal learning function has five parameters comprising total score, mean, fraction of zeroes, maximum continuous non-zero stretch, and variance.

One more embodiment of the present invention a method of identifying genes having evolutionary conserved peptide sequences which occur in ORFs of various genomes but not limited to genomes such as H. influenzae, M. genitalium, E. coli, B. subtilis, A. fulgidis, M. tuberculosis, T. pallidum, T. maritima, Synecho cystis, H. pylori and SARS-CoV.

In still another embodiment of the present invention the method identifies 169 novel genes identified in genomes of SARS-corona virus and H. influenzae, M. tuberculosis, H. pylori of SEQ IDs 1 to 169.

In further embodiment of the present invention, a method of the management of the diseases caused by the pathogenic organisms such as SARS-corona virus, H. influenzae, M. tuberculosis and H. pylori, said method comprising step of evaluation of the proposed drug candidate for inhibition of the functioning of one or more evolutionary conserved peptide sequences identified by the instant method and selected from a group comprising proteins of SEQ IDs 170 to 338 corresponding to the novel genes of SEQ IDs 1 to 169.

In yet another embodiment of the present invention the peptide library data may be taken from any organism but not specifically limited to those used in the invention.

Detailed Methodology:

The method has been described in five major steps (as shown in FIG. 1):
1. Generation of a peptide library
2. Artificial translation of a given genome into 6 reading frames
3. Conversion of each translated sequence into an alphanumeric sequence. (one corresponding to each reading frame)
4. Training of artificial neural network (ANN).

5. Deciphering genes using trained ANN.

1. Generation of Peptide Library

The method requires a reference peptide library to predict genes in a given genome. In the present invention, the applicants have used proteins from 56 completely sequenced prokaryotic genomes. The protein files for our database were obtained in FASTA format from ftp://ftp.ncbi.nlm.nih.gov/genomes. To prepare a peptide library for deciphering genes in a particular genome, the applicants exclude protein file(s) belonging to that particular species from our database in order to avoid any bias. For example, when analyzing E. coli-k12 genome the protein files corresponding to all strains of E. coli were excluded from the database to create the peptide library. This has been done to eliminate the signal that is obtained from peptides of that organism, which would be the case while analyzing a newly sequenced genome. This strengthens the method in terms of gene prediction on a newly sequenced genome for which annotated protein file is not available. While creating peptide library all possible overlapping heptapeptides have been taken care of by shifting the window by one amino acid. Redundant peptides were eliminated from the peptide library and each peptide is given an occurrence value based on number of discrete organisms in which it is present.

This occurrence value is a measure of conservation of a heptapetide in coding regions. Presence of a heptapeptide with high occurrence value in an ORF increases the likelihood of that ORF being a protein coding gene. In our algorithm, occurrence value of 9 or more is treated as 9 based on the assumption that if a heptapeptide is present in 9 or more than 9 different organisms' protein files, it can be considered as highly conserved heptapeptide. It is not worthwhile to use any higher value to further discriminate the amount of conservation.

The heptapeptide library database consists of two columns, first for heptapeptide sequence and second for score (occurrence value) of that heptapeptide. Heptapeptides are sorted in dictionary order. The peptide library database also retains other information about the heptapeptides, like the accession number and NCBI annotation of all proteins containing the particular heptapeptide. This can be utilized for putative function prediction of a given ORF. Same approach can be used for phylogenetic domain analysis also.

2. Artificial Translation of a Given Genome into 6 Reading Frames

Second step in the algorithm is artificial translation of the whole query genome in all six reading frames using a standard codon table. However user specified codon table may be used wherever necessary. Applicants used letter ‘z’ corresponding to the stop codons TTA, TAG and TGA, and letter ‘b’ for all triplets containing any non standard nucleotide(s) (K, N, W, R, and S etc.) while artificially translating the genome.

3. Conversion of Each Translated Sequence into an Alphanumeric Sequence (One Corresponding to Each Reading Frame)

The next step in our algorithm is to convert artificially translated amino acid sequence with stop codon (z) interruption, into an alphanumeric sequence. Applicants search each overlapping heptapeptide in the peptide library, assign a corresponding number (occurrence value), and append it to the alphanumeric sequence. If a heptapeptide is not present in the library applicants assign the number 0. If a heptapeptide begins with an amino acid corresponding to any of the start codon ATG, GTG and TTG applicants append character ‘s’ in the alphanumeric sequence. This will be helpful to detect the location of a probable start codon. In case a heptapeptide contains character ‘z’ applicants append a character ‘*’ corresponding to that heptapeptide. Thus consecutive seven ‘*’ (*******) in the alphanumeric sequence is a signal for stop codon. Applicants append ‘-’ character for any heptapeptide containing character ‘b’. This signals the presence of a non standard nucleotide character and conveys no information about sequence being a part of gene or non-gene. So, the alphanumeric sequence thus generated contain 13 characters viz. any integer (0-9), ‘s’, ‘*’, and ‘-’. In this way, applicants convert all six translated protein files into six alphanumeric sequences.

4. Training of Artificial Neural Network (ANN)

The neural network used here has a multi-layer feed-forward topology. It consists of one input layer, one hidden layer, and an output layer. This is a ‘fully-connected’ neural network where each neuron i is connected to each unit j of the next layer (FIG. 2). The weight of each connection is denoted by wᵢⱼ. The state Iᵢ of each neuron in the input layer is assigned directly from the input data, whereas the states of hidden layer neurons are computed by using the sigmoid function, hⱼ=1/(1+exp−λ(wⱼ₀+ΣwᵢⱼIᵢ)), where, wⱼ₀ is the bias weight, and λ=1.

The back propagation algorithm is used to minimize the differences between the computed output and the desired output. One thousand cycles (epochs) of iterations are performed. Subsequently, the epoch with minimum error in validation set is identified and the corresponding weights (wᵢⱼ) are assigned as the final weights for the ANN. The network trains on the training set, checks error and optimizes using the validation set through back propagation.

The ‘training set’ consists of 1610 E. coli-k12 NCBI listed protein coding genes and 3000 E. coli-k12 ORFs (a stretch of sequence of length more than 20 amino acids and having start codon, stop codon in the same frame) which have not been reported as genes (non-genes). The ‘validation set’ has 1000 known genes and 1000 non-genes from E. coli-k12, distinct from those used in the training set. The ‘test set’ contains another 1000 genes and 1000 non-genes from the same organism. For training of the ANN, genes and the non-genes are assigned a probability value of 1 and 0 respectively.

To train the neural network, first applicants convert all the E. coli-k12 genes and non-genes into corresponding alphanumeric strings by the method described above (steps 2 and 3). Here it is important to note that the alphanumeric sequences corresponding to a gene is number rich compared to the alphanumeric sequences corresponding to non-genes. To quantify this number richness of an alphanumeric sequence, five parameters derived from the alphanumeric sequence have been selected. These five parameters are as follows:

(i). Total Score

This is an algebraic sum of all the integers of a given alphanumeric sequence. Here rule of thumb is higher the score, more are the chances to qualify as a gene.

(ii). Fraction of Zeroes

Fraction of zeroes equals to total no. of zero characters in the alphanumeric sequence divided by total no. of characters in the sequence. More the fraction of zeros, lesser is the chance to qualify as a gene.

(iii). Mean

Mean equals to total score divided by total length of the sequence. Higher the Mean, more is the chance to qualify as a gene. Virtually this parameter seems same as a total score but it is important because this incorporates the length of the sequence also (score per unit length)

(iv). Variance

It is the variance of occurrence values about the mean occurrence value for the whole ORF.

(v). Length of the Maximum Continuous Non Zero Stretch

Higher the value of this parameter more is the chance to qualify as a gene. Consider a sequence region like ‘45’. Here, ‘4’ denotes a heptapeptide conserved in 4 organisms, and the succeeding ‘5’ denotes an overlapping heptapeptide conserved in 5 organisms. So if there exists at least one organism which is common between these two sets, eventually applicants have an octapeptide common between that organism and the query ORF. This raises our confidence level in prediction of the coding region. For example, sequence ‘s45467000000*******’ is more likely to be a gene when compared to sequence ‘s40540607000*******’. This is because there are greater chances of presence of conserved longer peptide in the first sequence. Value of the parameter is 5 for first string and 2 for second one. However, other parameters used in the algorithm can not discriminate between these two sequences.

While calculating these parameters from the alphanumeric sequences, characters such as ‘s’, ‘*’ and ‘-’ have been excluded.

To find an optimum combination, the neural network is trained using all the five parameters together. Parameters corresponding to alphanumeric sequences of genes and non-genes are calculated. The training, validation and test sets contain 6 columns, first 5 columns contains values of the 5 parameters and the last column contains the number ‘1’ for genes and the number ‘0’ for non-genes.

The number of neurons in the input layer was equal to the number of input data points. The optimal number of neurons in the hidden layer was determined by hit and trial while minimizing the error at the best epoch for the network. Computer program to compute all 5 parameters and for the artificial neural network are written in C and executed on a PC under Red Hat Linux version 7.3 or 8.0.

Training of the ANN (step 4 of the algorithm) is generally executed only once, and the same trained neural network can be utilized to execute the method on any prokaryotic genome. Although if applicants use organism specific training set, results might improve in some cases, but it would be marginal. This is because our method predicts gene on the basis of the number distribution of the alphanumeric sequence of an ORF. So the gene prediction is more dependent on the peptide library used rather than training set.

5. Deciphering Genes Using Trained ANN

While creation of peptide library (step 1) and training of ANN (step 4) are considered as preparatory phases for executing the method of invention, step 2 and step 3 are mandatory for each genome sequence. After translating computationally a genome into all six reading frames and converting them into six alphanumeric sequences, deciphering genes using ANN is executed. This step can be further divided into following five sub-steps:
1. Breaking of all the six alphanumeric sequences into possible ORFs. (all possible fragments starting with ‘s’ and ending with ‘*’)
2. Calculate all the five parameters (total score, fraction of zeroes, mean, variance, and length of maximum continuous non zero stretch) for all possible ORFs (all the alphanumeric string sequences between ‘s’ and ‘*’)
3. Calculate the probability of the ORF corresponding to a given alphanumeric string as a protein coding gene, using the trained ANN.

4. Filter out the protein coding ORFs from the non coding ones by using a cutoff probability value.

5. Remove all the encapsulated protein coding regions (Shibuya, T. and Rigoutsos, I., 2002).
If two ORFs are predicted in distinct translation frames, such that one's span completely encapsulates other, it is a commonly believed that only one of them can be an actual gene. In this case the applicants report the ORF with a higher probability value as a gene. In case of same probability value applicants take longer ORF as a gene.

The method of the invention predicts a probability value corresponding to a query ORF being a protein coding region. The training of ANN is done using a sigmoid learning function with =1 (probability ‘1’for genes and ‘0’ for non-genes); therefore most of the time this probability value lies either below 0:1 or above 0.9. Due to this any cutoff value lying between 0.1 and 0.9 generate very similar results. In our analysis applicants use a default cutoff value of 0.5. It's important to note that the method does not require a trade-off between sensitivity and specificity because the choice of cut-off probability has no major consequences on the results.

Other and further aspects, features and advantages of the present invention will be apparent from the following description of the presently preferred embodiments of the invention given for the purpose of disclosures.

Brief Description of the Computer Programs:

1. File Name: genedcodchr.cxx

Application: Translation of nucleotide sequence (FASTA file format) into 6 hypothetical polypeptides in 6 respective frames.

Input format : <Program_name> <Nucleotide_file> <Output1> <Output2>

| | |
|---|---|
| <frame> e.g., | ./genedcodchr ecoli.fna pf1 pr1 0 |

| | |
|---|---|
| Output | format: |

AGTFYRYmGHVNMKIYTASLPTYRYGYFSHRED.....HGOIEKSDWEzDFGTRE

2. File Name: searchchr.cxx

Application: Converts the polypeptide file into an alphanumeric sequence through a heptapetide library (given as an input) search.

```
  Input format :< Program_name> 7 <peptide library file name>
  out Y <Input1>
```

| | |
|---|---|
| <Input2> | <Output1> <Output 2> |

| | |
|---|---|
| e.g., | ./searchchr 7 ecoli.peplib out Y pf1 pr1 bf1 br1 |

| | |
|---|---|
| Output | format: |

s1124500001090003000020000023000000000*******0001000..........

3. File Name: cutf.c
Application: Cuts all possible ORFs (i.e., all ‘s’ to ‘*’ regions) from the alphanumeric sequence of forward strand and generates a file containing locations of all the ‘s’ in alphanumeric sequence.
Input format:<Program_name><Input file name><Output1><Output2>e.g./cutf bf1 unknown_bf1 bf1_location
Output format: output1—s1111000s00000000563*, output2—starting locations of ‘s’ in a column.
   4. File Name: cutr.c

Application: Cuts the all possible ORFs (all ‘s’ to ‘* regions) from the reverse strand's alphanumeric sequences and produces a file which contains the starting locations in alphanumeric sequence file for all 3 forward frames corresponding to all ORFs.

| Input format :< Program_name> <Input file name> <Output1> | |
|---|---|
| <Output2> | |
| e.g. | ./cutr br1 unknown_br1 br1_location |

Outputformat:
output1-*010340000222200067900000s000001000200s00230000s,

output2—starting location of ‘s’
   5. File Name: stat.c

Application: Calculates the five parameters: fraction of zeros, mean, total score, length of maximum continuous stretch, and variance for a given alphanumeric sequence.

| Input format :< Program_name> <Input file name><Output> 1 | |
|---|---|
| e.g. | ./stat unknown_bf1 bf1.data 1 |

| | | | | |
|---|---|---|---|---|
| Output format: 0.334 | 3.2 | 48 | 15 | 0.452 1 |

6. File Name: train .c

Application: Training of Artificial Neural Network (single hidden layer, 1 input and 1 output layer) with feed forward back propagation algorithm and using sigmoid (=1) as a learning function.

Input format :< Program_name> <Input specification file
name> <Input1>

<Input2> <Input3> > output

| | |
|---|---|
| e.g. | ./train train.spec.fast trainset.data |

validateset.data testset.data >

train.net

Output format: output containing the final neural network wieghts in a single column.
   7. File Name: recognize.c

Application: Recognizes a given pattern on the basis of trained weights and generates a probability value as output.

Input tormat :< Program_name> <Input specification file
name> <Input1>

<Input2>

| <Output> | |
|---|---|
| e.g. | ./recognize recognize.spec bf1.data train.net |
| f1.out | |

Output format: pat1 probability <value>

8. File Name: Filter_prediction.c

Application: Filters out the completely overlapping ORFs in same frame based on probability and length parameter.

| Input format :< Program_name> <Input1> <Input2> <Output> | |
|---|---|
| e.g. | ./Filter_prediction f1.out unknown_bf1 bf1.out.res |

| | | |
|---|---|---|
| Output format: pat1 probability | <value> | <integer string> |

9. File Name: locationf.c

Application: Filters out the genes of length<20 amino acids, and reports starting location of the remaining ones with the alphanumeric sequence for all 3 forward frames.

| Input format :< Program_name> <Input1> <Output> <Input2> | |
|---|---|
| e.g. | ./locationf bf1.out.res bf1.out.res1 bf1_location |

Output format:<Pattern No> <Probability value> <integer string>
<Start> <End>

10. File Name: locationr.c

Application: Filters out the genes of length<20 amino acids, and reports starting location of the remaining ones with the alphanumeric sequence for all 3 reverse frames.

| Input format :< Program_name> <Input1> <Output> <Input2> | |
|---|---|
| e.g. | ./locationr br1.out.res br1.out.res1 br1_location |

Output format:<Pattern No> <Probability value> <integer string>
<Start> <End>

11. File Name: finalf.c

Application: Converts the start and end locations of the alphanumeric sequence into the corresponding genome locations for 3 forward frames.

| Input format :< Program_name> <Input1> <Input2> <Input3> | |
|---|---|
| <Output> | |
| e.g. | ./finalf bf1.out.res1 bf2.out.res1 bf3.out.res1 |

Final_outputf
Output format:<Start> <End> <frame> <length> <Probability
value> <integer

string>

12. File Name: finalr.c

Application: Converts the start and end locations of the alphanumeric sequence into the corresponding genome locations for 3 reverse frames.

| Input format :< Program_name> <Input1> <Input2> <Input3> | |
|---|---|
| <Output> | |
| e.g. | ./finalf br1.out.res1 br2.out.res1 br3.out.res1 |

Final_outputr
Output format:<Start> <End> <frame> <length> <Probability value>
<integer

string>

13. File Name: sort.c
File Name: sort.c

Applications: Prints the finally predicted genes into descending order along the genome start location.

Input format :< Program_name> <Input1> <Input2> <Input3> <Output>

| | | | |
|---|---|---|---|
| e.g. | ./sort | Final_outputf | Final_outputr |

OUTPUTF_with_encap
OUTPUTR_with_encap OUTPUT
Output format:<Start> <End> <Probability value>

14. File Name: removeencap.c

Application: Removes encapsulated genes found in other five frames.

| Input format :< Program_name> <Input1> <Input2> <Input3> | | |
|---|---|---|
| <Output> | | |
| e.g. | ./removeencap | OUTPUTF_with_encap |

OUTPUTR_with_encap

OUTPUT OUTPUTF OUTPUTR
Output format:<Start> <End> <frame> <length> <Probability
value> <integer

string>

The present invention relates to a novel computer based method for predicting protein coding DNA sequences useful as drug targets. In this method occurrence of oligopeptide signatures have been used as probes. The method is versatile and does not necessarily require organism specific training set for the Artificial Neural Network. The method is not only dependent on statistical analysis but also integrates with the biological information that is retained in the conserved peptides, which withstood evolutionary pressure. Logical extension of the method will be to predict protein coding DNA sequences (exons) in eukaryotic genomes.

### BRIEF DESCRIPTION OF THE ACCOMPANYING DRAWINGS

FIG. 1 shows a logic circuit of GeneDecipher.

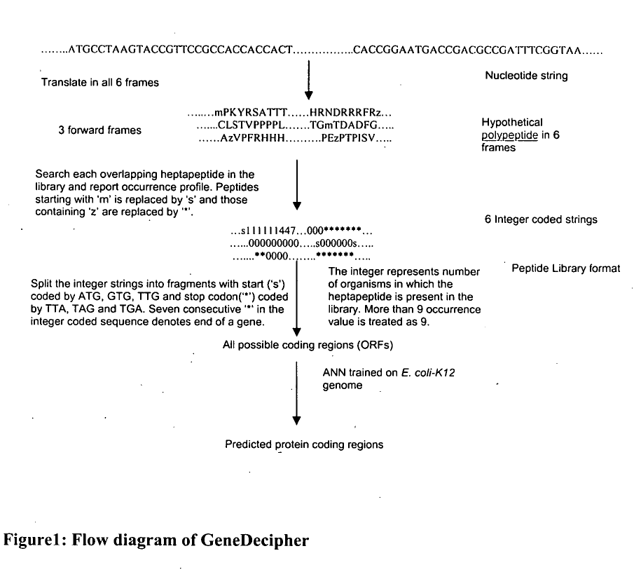

FIG. 2 shows a architecture of neural network.

FIG. 3 shows analysis of results of GeneDecipher on 10 organisms.

The particulars of the organisms used for the invention comprising name, strain, accession number and other details are given below.

| | | | | | Date of |
|---|---|---|---|---|---|
| S. No. | Genome | Strain | Accession Number | Total Base Sequences | Completion |
| 1 | H. Influenzae | Rd | NC_000907 | 1830138 | Sep. 30, 1996 |

Fleischmann, R. D. et. al Science 269 (5223), 496-512 (1995)

| | | | | | |
|---|---|---|---|---|---|
| 2 | M. Genitalium | — | NC_000908 | 580074 | Jan. 8, 2001 |

Fraser, C. M., et. al Science 270 (5235), 397-403 (1995

| | | | | | |
|---|---|---|---|---|---|
| 3 | E. coli | K-12 | NC_000913 | 4639221 | Oct. 15, 2001. |

Blattner, F. R. et. al Science 277 (5331), 1453-1474 (1997)

| | | | | | |
|---|---|---|---|---|---|
| 4 | B. Subtilis | 168 | NC_000964 | 4214814 | Nov. 20, 1997 |

Kunst, F. et. al Nature 390 (6657), 249-256 (1997)

| | | | | | |
|---|---|---|---|---|---|
| 5 | A. Fulgidis | DSM 4304 | NC_000917 | 2178400 | Dec. 17, 1997 |

Klenk, H. P. et. al Nature 390 (6658), 364-370 (1997)

| | | | | | |
|---|---|---|---|---|---|
| 6 | M. Tuberculosis | H37RV | NC_000962 | 4411529 | Sep. 7, 2001 |

Cole, S. T. et. al Nature 393 (6685), 537-544 (1998)

| | | | | | |
|---|---|---|---|---|---|
| 7 | T. Pallidum | — | NC_000919 | 1138011 | Sep. 7, 2001 |

Fraser, C. M., et. al Science 281 (5375), 375-388 (1998)

| | | | | | |
|---|---|---|---|---|---|
| 8 | T. Maritima | — | NC_000853 | 1860725 | Sep. 10, 2001. |

Nelson, K. E. et. al Nature 399 (6734), 323-329 (1999)

| | | | | | |
|---|---|---|---|---|---|
| 9 | Synecho cystis | PCC6803 | NC_000911 | 3573470 | Oct. 30, 1996 |

Kaneko, T. et. al DNA Res. 3(3), 109-136 (1996)

| | | | | | |
|---|---|---|---|---|---|
| 10 | H. Pylori | 26695 | NC_000915 | 1667867 | Sep. 7, 2001 |

Tomb, J. -F. et. al Nature 388 (6642), 539-547 (1997)

The following examples are given by way of illustration of the present invention and should not be construed to limit the scope of the present invention.

### EXAMPLE 1

Conversion of DNA Sequence into Alphanumeric Sequence

The purpose of this module in our software is to translate computationally the whole query genome (DNA sequence) in all six reading frames using a specified codon table. Applicants used letter ‘z’ corresponding to the stop codons TTA, TAG and TGA, and letter ‘b’ for all triplets containing any non standard nucleotide(s) (K, N, W, R, and S etc.) while artificially translating the genome. Subsequently the translated genome sequence is converted computationally into an alphanumeric sequence ([0-9], ‘s’, ‘*’, and ‘-’). Applicants search each overlapping heptapeptide in the peptide library, assign a corresponding number (occurrence value), and append it to the alphanumeric sequence. If a heptapeptide is not present in the library applicants assign the number 0. If a heptapeptide begins with an amino acid corresponding to any of the start codon ATG, GTG and TTG Applicants append character ‘s’ in the alphanumeric sequence. This will be helpful to detect the location of a probable start codon. In case a heptapeptide contains character ‘z’ applicants append a character ‘*’ corresponding to that heptapeptide. Thus consecutive seven ‘*’ (*******) in the alphanumeric sequence is a signal for stop codon. Applicants append a ‘-’ character for any heptapeptide containing character ‘b’. This signals the presence of a non-standard nucleotide character.

The aforementioned conversion is further elaborated with the help of following six sequences.

| SEQ ID No. 12 | | | | | |
|---|---|---|---|---|---|
| GDC_HINF_243018 | 243018 | 243215 | 65 | + | Cell wall-associated |
| | | | | | hydrolase |

>gi_GDC_HINF_243018
GTGATGAGCCGACATCGAGGTGCCAAACACCGCCGTCGATATGAACTCTTGGG
CGGTATCAGCCTGTTATCCCCGGAGTACCTTTTATCCGTTGAGCGATGGCCCTT
CCATTCAGAACCACCGGATCACTATGACCTACTTTCGTACCTGCTCGACTTGTC
TGTCTCGCAGTTAAGCTTGCTTATACCATTGCACTAA

Computationally Translated Protein Sequence

>gi_GDC_HINF_243018
VMSRHRGAKHRRRYELLGGISLLSPEYLLSVERWPFHSEPPDHYDLLSYLLDLSVSQLSLLIPLH

Computationally Generated Alphanumeric Sequence

ss10000000000001s03111431000000000000000000110000100s001030*

| SEQ ID No. 4 | | | | | |
|---|---|---|---|---|---|
| GDC_HINF_170553 | 170553 | 170732 | 59 | − | dicarboxylate transport protein |
| | | | | | homolog HI0153 |

>gi_GDC_HINF_170553
GTGTTTATGCTTTATTTAGAATTTTTATTTTTACTATTAATGCTCTATATCGGTA
GCCGTTACGGCGGTATCGGATTAGGTGTTGTTTCTGGTATCGGTCTTGCTATCG
AGGTTTTCGTATTTCGTATGCCAGTGGGGAAGCACCGATTGATGTTATGCTTAT
CATTCTTGCAGTGGTGA

Computationally Translated Protein Sequence

>gi_GDC_HINF_170553
VFMLYLEFLFLLLMLYIGSRYGGIGLGVVSGIGLAIEVFVFRMPVGKHRLMLCLSFLQW

Computationally Generated Alphanumeric Sequence

s0s1131231142s1111445232254238000000000000s0s0000ss00*

| SEQ ID No. 73 | | | | | |
|---|---|---|---|---|---|
| GDC_MTUB_688806 | 688806 | 689060 | 84 | + | MCE-FAMILY PROTEIN |
| | | | | | MCE2B |

>gi_GDC_MTUB_688806
TTGCTGCACAGCAGCTTCGGGCACCTCGAGGGCATCCAGCAGCCGCTCATAGA
CGAGCTGGCAGAACTCGACCACGTGTTGGGCAAGCTGCCGGACGCCTACCGGA
TCATCGGCCGCGCCGGCGGCATATACGGTGACTTCTTCAACTTCTATCTGTGTG
ACATCTCACTGAAAGTCAACGGATTACAGCCTGGAGGTCCGGTACGCACCGTC
AAGTTGTTCGGCCAGCCGACCGGCAGGTGCACACCGCAATGA

Computationally Translated Protein Sequence

>gi_GDC_MTUB_688806
LLHSSFGHLEGIQQPLIDELAELDHVLGKLPDAYRIIGRAGGIYGDFFNFYLCDISLK
VNGLQPGGPVRTVKLFGQPTGRCTPQ

Computationally Generated Alphanumeric Sequence

s000000000110110530100000ss000000000000100000000000000000001111210000000s00100*

| SEQ ID No. 92 | | | | | |
|---|---|---|---|---|---|
| GDC_MTUB_1286282 | 1286282 | 1286587 | 101 | − | pterin-4-alpha- |
| | | | | | carbinolamine |
| | | | | | dehydratase |

>gi_GDC_MTUB_1286282
GTGACGGTATACCGTCGAGGTATGGCTGTGTTAACGGATGAGCAGGTCGACGC
CGCACTGCACGACCTCAACGGCTGGCAGCGCGCCGGTGGTGTCCTGCGTAGGT
CAATCAAGTTTCCGACGTTTATGGCCGGTATCGACGCCGTACGCCGGGTGGCC
GAGCGAGCCGAGGAGGTAAATCATCATCCGGACATCGATATCCGTTGGCGAAC
AGTAACTTTCGCGCTGGTTACGCATGCGGTAGGTGGTATCACGGAAAACGACA
TTGCGATGGCGCACGATATCGACGCAATGTTTGGGGCCTAA

Computationally Translated Protein Sequence

>gi_GDC_MTUB_1286282
VTVYRRGMAVLTDEQVDAALHDLNGWQRAGGVLRRSIKFPTFMAGIDAVRRVA
ERAEEVNHHPDIDIRWRTVTFALVTHAVGGITENDIAMAHDIDAMFGA

Computationally Generated Alphanumeric Sequence

s000000s0s21110001000000300000000011000000s01031100s00020000110000000030000000013310000000s0001*

| SEQ ID No. 49 | | | | | |
|---|---|---|---|---|---|
| GDC_HPYL_583607 | 583607 | 583876 | 89 | + | probable DNA |
| | | | | | helicase |

>gi_GDC_HPYL_583607
TTGATGGAATTTGATGTTACCATCATAGATGAGACAGGCAGGGCCACAGCACC
AGAAATCTTGATTCCTGCACTTCGCACTAAAAAACTGATCTTAATAGGCGATC
ACAACCAGCTCCCACCTAGCATTGATAGGTACCTCCTAGAACAATTAGAGAGC
GATGATATTCAAAACTTGGATGCCATTGATCGCCAATTATTGGAAGAGAGTTT
TTTTGAAAATCTCTATAAGTATATTCCAGAGAGTAATAAGGCCATGCTTAATG
AGTAA

Computationally Translated Protein Sequence

>gi_GDC_HPYL_583607
LMEFDVTIIDETGRATAPEILIPALRTKKLILIGDHNQLPPSIDRYLLEQLESDDIQNL
DAIDRQLLEESFFENLYKYIPESNKAMLNE

Computationally Generated Alphanumeric Sequence

ss001000000001000000s0000011000020000000000030310000000002s0003020s0000000000000000*

| SEQ ID No. 54 | | | | | |
|---|---|---|---|---|---|
| GDC_HPYL_954846 | 954846 | 955217 | 123 | − | PHOSPHOTRANSACETY |
| | | | | | LASE |

>gi_GDC_HPYL_954846
GTGAGCCTGGTTTCAAGCGTGTTTTTAATGTGTTTAGACACTCAAGTGCTAGTC
TTTGGGGATTGCGCGATTATCCCTAACCCTAGCCCTAAAGAATTAGCCGAGAT
CGCTACCACTTCCGCACAAACCGCCAAGCAATTCAATATTGCGCCTAAAGTGG
CCTTGCTTTCTTATGCGACAGGCGATTCCGCTCAAGGCGAAATGATAGACAAA
ATCAACGAAGCTTTAACAATCGCTCAAAAGTTGGATCCCCAATTAGAAATTGA
TGGCCCCTTACAATTTGACGCTTCCATTGATAAAAGCGTAGCCAAGAAAAAAT
GCCTAACAGCCAAGTGGCTGGGCAAGCTAGCGTTTTTATTTTCCCGGATTTAA

Computationally Translated Protein Sequence

>gi_GDC_HPYL_954846
VSLVSSVFLMCLDTQVLVFGDCAIIPNPSPKELAEIATTSAQTAKQFNIAPKVALLS
YATGDSAQGEMIDKINEALTIAQKLDPQLEIDGPLQFDASIDKSVAKKKCLTAKWL
GKLAFLFSRI

Computationally Generated Alphanumeric Sequence
s80000s00s00002s200222000000003100000000000000000010s0s100000000000s0000000100000s00000000000000000000000000030000010*

### EXAMPLE 2

Training of Artificial Neural Network (ANN)

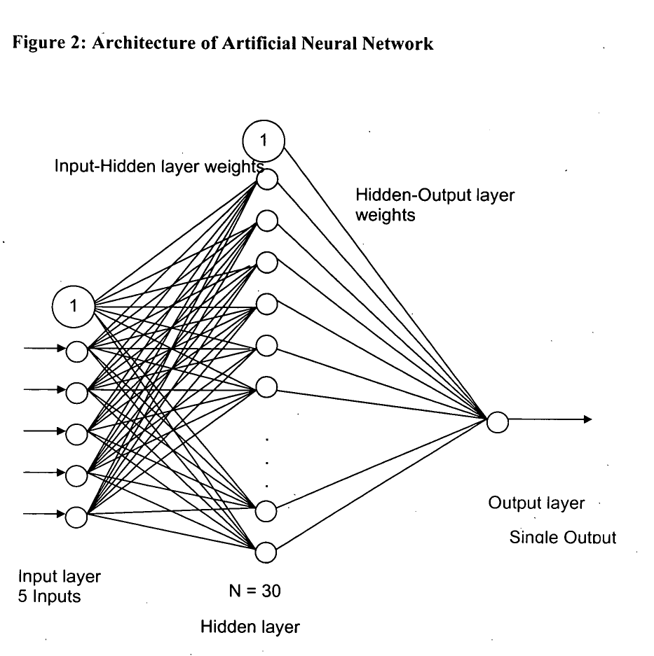

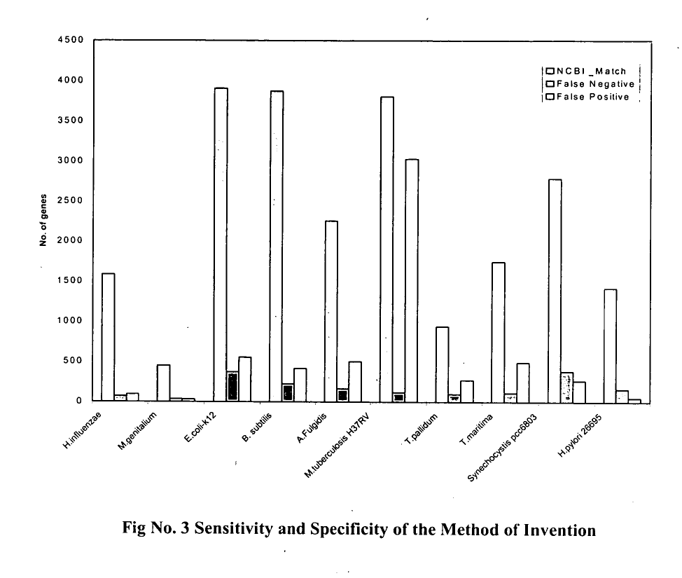

The purpose of this module in the software is to train the designed neural network (FIG. 2) with a specified no. of genes and non-genes. In this example the training set consists of 1610 E. coli-k12 NCBI listed protein coding genes and 3000 E. coli-k12 ORFs which have not been reported as genes (non-genes). The validation set has 1000 known genes and 1000 non-genes from E. coli-k12, distinct from those used in the training set. The test set contains another 1000 genes and 1000 non-genes from the same organism. For training of the ANN, genes and the non-genes are assigned a probability value of 1 and 0 respectively. To train the neural network, first applicants convert all the E. coli-k12 genes and non-genes into corresponding alphanumeric strings by the method described above (steps 2 and 3). Samples of two E. coli-k12 genes and two non-genes in alphanumeric sequence format are shown in FIG. 3. Here it is important to note that the alphanumeric sequences corresponding to a gene is number rich compared to the alphanumeric sequences corresponding to non-genes. This supports our hypothesis. To quantify this number richness of an alphanumeric sequence, five parameters derived from the alphanumeric sequence have been selected. These five parameters are as follows:

Total Score (algebraic sum of all the integers of a given alphanumeric sequence), Fraction of zeroes (total no. of zero characters in the alphanumeric sequence divided by total no. of characters in the sequence), Mean (total score divided by total length of the sequence), Variance (variance of occurrence values about the mean occurrence value for the whole ORF), Length of the maximum continuous non zero stretch (represents the occupancy of uninterrupted non-zero numbers in a sequence) as explained in table 1(a) and 1(b).

TABLE 1(a)
Training of ANN (genes)

| | | | | Biggest | | |
|---|---|---|---|---|---|---|
| S. | Fraction | Total | | Continuous | | |
| No | of Zeros | Score | Average | stretch | Variance | Probability |
| 1 | 0.663116 | 587 | 0.7816 | 19 | 2.10146 | 1 |
| 2 | 0.693950 | 214 | 0.7616 | 18 | 2.43068 | 1 |
| 3 | 0.597436 | 412 | 1.0590 | 13 | 3.16832 | 1 |
| 4 | 0.898876 | 12 | 0.1348 | 4 | 0.20654 | 1 |

TABLE 1(b)
Training of ANN (Non-genes)

| | | | | Biggest | | |
|---|---|---|---|---|---|---|
| S. | Fraction | Total | | Continuous | | |
| No | of Zeros | Score | Average | stretch | Variance | Probability |
| 1 | 0.946429 | 3 | 0.0536 | 2 | 0.05070 | 0 |
| 2 | 1.000000 | 0 | 0.0000 | 0 | 0.00000 | 0 |
| 3 | 0.955556 | 2 | 0.0444 | 1 | 0.04247 | 0 |
| 4 | 0.956522 | 2 | 0.0435 | 1 | 0.04159 | 0 |

While calculating these parameters from the alphanumeric sequences characters ‘s’, ‘*’ and ‘-’ have been excluded. To determine the contribution of each parameter towards discriminating genes from non-genes, the neural network is trained using all the five parameters together. Parameters corresponding to alphanumeric sequences of genes and non-genes are calculated. The training, validation and test sets contain 6 columns, first 5 columns contains values of the 5 parameters and the last column contains the number ‘I’ for genes and the number ‘0’ for non-genes.

### EXAMPLE 3

The applicants have analyzed 10 prokaryotic genomes using the method of invention. Efficiency of the method has been defined as percentage of the NCBI listed protein coding regions predicted by said method. All the encapsulated protein coding regions have been eliminated automatically by a specifically developed program. The method is able to predict on an average 92.7% of the NCBI listed genes with a standard deviation of 2.8%. Both sensitivity and specificity values of the method are high except in M. tuberculosis H37RV genome (as shown in FIG. No. 3).

### EXAMPLE 4

Prediction of Start Site of Protein Coding DNA Sequences

Correct start site prediction rate of the method of invention varies from 49.5% in M. tuberculosis H37Rv (where specificity is also least) to 81.1% in H. pylori 26695. The applicants method decides start location based on the presence of start codon plus conservation of the surrounding heptapeptides. This method can also be utilized to predict the start site of a query protein coding DNA sequences predicted by some other method. This can be done by simply converting the protein sequence into corresponding integer sequence and then deciding the valid start site ‘s’ on the basis of surrounding heptapeptides. The applicants report three such cases from E. coli K-12 genome (two from the forward strand and one from the reverse strand), to exemplify the start site prediction (as shown below).

In prediction of start site there is a trade-off between number richness and length of the ORF. In Case 1 (PID 16132273), the start location of the gene has been shifted from location 85540 to 85630 by NCBI. By visual inspection of the integer sequences corresponding to this gene it is evident that earlier there was a region after ‘s’ which was full of zeroes; or in other terms not a number rich region (bold region in Case 1 of figure shown below). The start site has now been shifted so that it now lies before a number rich region as predicted by the said method of invention. Case 2 is an example of 5′ upstream shifting of the start codon because there is a number rich region (‘2011111’ and one ‘3’ and one ‘2’) upstream of this start codon. So this has been shifted to location 4611050 from 4611194. Case 3 is another example of shifting of start site in the reverse strand where there is a number rich region (‘16531311’ and many other numbers in the string) upstream of the earlier NCBI start location.

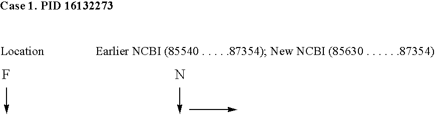

s0s0000000000000s000000000s000s2ss4222s111000000000999922224210000s00s40004
466442223s0s0120000000177s9999855553239888440s001111000113002s1116311112ss
22222s430100000000100s0100000639977100011100100000001000000000s2000010030
000011110111100000161171000000000s201s12s0000002ss10000000001099s76s621110
0s0s0000s00014444441111100000000000234331211000s033221s000000014s000s00000
002000000000001110000000000000000000s000001s000000s48976531s11111100012234
59999999s92554010010s0s0002s2236667778s75221001s000s000ss00000066ss11111s32
11100000s000002204332110000000000210010010000s00000s11000000354211s000000s
00s22*******

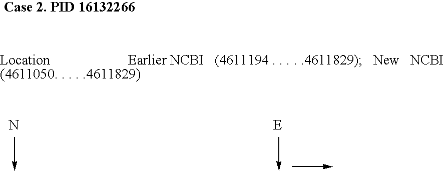 s00020111110000000000000300000000020000010000030ss000000001110s0s000ss0000
0s102110000000100ss3s2000000000000000000000100021100011s110000000000s00000
000001s10100000010100002222222000000000000000010321002s3321111s1101111001
0000000s00s000s00101010100s00000*******

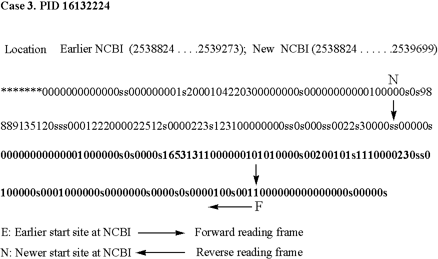

### EXAMPLE 5

Prediction of Protein Coding DNA Sequences

The method is utilized for prediction of protein coding DNA sequences for various genomes in a publicly available database (NCBI) by employing the following steps:
i) generating computationally overlapping peptide libraries from all the protein sequences of the selected organisms available at http://www.ncbi.nlm.nih.gov,
ii) sorting computationally the peptides of length ‘N’ obtained as above, alphabetically, according to single letter amino acid code,
iii) cataloging every peptide and their unique occurrence different organisms,
iv) converting DNA sequence to alphanumeric sequence using peptide library obtained from steps 1 and 2,
v) retrieving all possible open reading frames (ORFs) from the alphanumeric sequence,
vi) training of the modified neural network for discriminating protein coding and non-coding DNA sequences,
vii) predicting DNA coding sequences in the open reading frames (obtained in step 4) using trained neural network,
viii) removing the encapsulated protein coding DNA sequences (genes within genes)

Using the steps of the invention the inventors have arrived at disclosure of novel 169 genes from the genomes of organisms selected from SARS-corona virus, H. influenzae, M. tuberculosis, and H. pylori as detailed in the table 2. The Table No. 2 provides the said novel genes in the sequence of SEQ ID No. 1 to SEQ ID No. 169.

TABLE 2

| | | | | | | |
|---|---|---|---|---|---|---|
| 1 | GDC_HINF_{—} | 5641 | 6273 | 210 | + | Formate dehydrogenase major |
| | 5641 | | | | | subunit |
| 2 | GDC_HINF_{—} | 6322 | 8748 | 808 | + | Formate dehydrogenase major |
| | 6322 | | | | | subunit |
| 3 | GDC_HINF_{—} | 124181 | 124378 | 65 | + | Cell wall-associated hydrolase |
| | 124181 | | | | | |
| 4 | GDC_HINF_{—} | 170553 | 170732 | 59 | − | dicarboxylate transport protein |
| | 170553 | | | | | homolog HI0153 |
| 5 | GDC_HINF_{—} | 231874 | 232173 | 99 | + | type I restriction system |
| | 231874 | | | | | adenine methylase |
| 6 | GDC_HINF_{—} | 232170 | 232991 | 273 | + | type I restriction system |
| | 232170 | | | | | adenine methylase |
| 7 | GDC_HINF_{—} | 232813 | 233139 | 108 | + | type I restriction system |
| | 232813 | | | | | adenine methylase |
| 8 | GDC_HINF_{—} | 233190 | 233393 | 67 | + | Type I restriction enzyme |
| | 233190 | | | | | EcoprrI M protein |
| 9 | GDC_HINF_{—} | 235441 | 235932 | 163 | + | prrD protein homolog |
| | 235441 | | | | | |
| 10 | GDC_HINF_{—} | 235913 | 238519 | 868 | + | Type I restriction enzyme |
| | 235913 | | | | | EcoR124II R protein |
| 11 | GDC_HINF_{—} | 240336 | 241379 | 347 | − | Aerobic respiration control |
| | 240336 | | | | | sensor protein |
| 12 | GDC_HINF_{—} | 243018 | 243215 | 65 | + | Cell wall-associated hydrolase |
| | 243018 | | | | | |
| 13 | GDC_HINF_{—} | 274892 | 276853 | 653 | − | Adhesion and penetration |
| | 274892 | | | | | protein precursor |
| 14 | GDC_HINF_{—} | 276992 | 279121 | 709 | − | Adhesion and penetration |
| | 276992 | | | | | protein precursor |
| 15 | GDC_HINF_{—} | 370413 | 370808 | 131 | + | NapA |
| | 370413 | | | | | |
| 16 | GDC_HINF_{—} | 370747 | 372912 | 721 | + | NapA |
| | 370747 | | | | | |
| 17 | GDC_HINF_{—} | 628407 | 628604 | 65 | − | Cell wall-associated hydrolase |
| | 628407 | | | | | |
| 18 | GDC_HINF_{—} | 654365 | 655015 | 216 | − | Probable D-methionine |
| | 654365 | | | | | transport system permease |
| 19 | GDC_HINF_{—} | 661444 | 661641 | 65 | − | Cell wall-associated hydrolase |
| | 661444 | | | | | |
| 20 | GDC_HINF_{—} | 737160 | 737297 | 45 | + | glycerophosphodiester |
| | 737160 | | | | | phosphodiesterase |
| 21 | GDC_HINF_{—} | 775792 | 775989 | 65 | − | Cell wall-associated hydrolase |
| | 775792 | | | | | |
| 22 | GDC_HINF_{—} | 848166 | 848678 | 170 | − | ribosomal protein |
| | 848166 | | | | | |
| 23 | GDC_HINF_{—} | 928073 | 929080 | 335 | + | Peptidase B (Aminopeptidase |
| | 928073 | | | | | B) |
| 24 | GDC_HINF_{—} | 929037 | 929402 | 121 | + | Peptidase B (Aminopeptidase |
| | 929037 | | | | | B) |
| 25 | GDC_HINF_{—} | 1018846 | 1021371 | 841 | − | Isoleucyl-tRNA synthetase |
| | 1018846 | | | | | |
| 26 | GDC_HINF_{—} | 1021582 | 1021683 | 33 | − | Isoleucyl-tRNA synthetase |
| | 1021582 | | | | | |
| 27 | GDC_HINF_{—} | 1082407 | 1082514 | 35 | − | protein V6, truncated - |
| | 1082407 | | | | | Haemophilus influenzae |
| 28 | GDC_HINF_{—} | 1144501 | 1145004 | 167 | − | PnuC transporter |
| | 1144501 | | | | | |
| 29 | GDC_HINF_{—} | 1279189 | 1279935 | 248 | − | Peptide chain release factor 2 |
| | 1279189 | | | | | (RF-2) |
| 30 | GDC_HINF_{—} | 1347200 | 1347445 | 81 | + | putative ABC transport protein |
| | 1347200 | | | | | |
| 31 | GDC_HINF_{—} | 1347942 | 1348478 | 178 | + | putative iron compound ABC |
| | 1347942 | | | | | transporter |
| 32 | GDC_HINF_{—} | 1476415 | 1476615 | 66 | − | PstB |
| | 1476415 | | | | | |
| 33 | GDC_HINF_{—} | 1476557 | 1477183 | 208 | − | PstB |
| | 1476557 | | | | | |
| 34 | GDC_HINF_{—} | 1505851 | 1506048 | 65 | − | terminase large subunit |
| | 1505851 | | | | | |
| 35 | GDC_HINF_{—} | 1524561 | 1525421 | 286 | − | ThiI |
| | 1524561 | | | | | |
| 36 | GDC_HINF_{—} | 1568974 | 1569300 | 108 | + | DNA-binding protein rdgB |
| | 1568974 | | | | | homolog |
| 37 | GDC_HINF_{—} | 1586944 | 1587765 | 273 | + | putative tail protein |
| | 1586944 | | | | | |
| 38 | GDC_HINF_{—} | 1594339 | 1594854 | 171 | − | NifC |
| | 1594339 | | | | | |
| 39 | GDC_HINF_{—} | 1634710 | 1636722 | 670 | + | Probable hemoglobin and |
| | 1634710 | | | | | hemoglobin-haptoglobin |
| 40 | GDC_HINF_{—} | 1638626 | 1639372 | 248 | − | Putative integrase/recombinase |
| | 1638626 | | | | | HI1572 |
| 41 | GDC_HINF_{—} | 1639409 | 1639726 | 105 | − | Putative integrase/recombinase |
| | 1639409 | | | | | HI1572 |
| 42 | GDC_HINF_{—} | 1660491 | 1662080 | 529 | − | Cell division protein ftsK |
| | 1660491 | | | | | homolog |
| 43 | GDC_HINF_{—} | 1807963 | 1808859 | 298 | − | adhesin homolog HI1732 |
| | 1807963 | | | | | |
| 44 | GDC_HINF_{—} | 1817220 | 1817417 | 65 | + | Cell wall-associated hydrolase |
| | 1817220 | | | | | |
| 45 | GDC_HPYL_{—} | 51094 | 51432 | 112 | − | putative HP0052-like protein |
| | 51094 | | | | | |
| 46 | GDC_HPYL_{—} | 155367 | 156164 | 265 | − | 2-oxoglutarate/malate |
| | 155367 | | | | | translocator |
| 47 | GDC_HPYL_{—} | 447632 | 447850 | 72 | − | Cell wall-associated hydrolase |
| | 447632 | | | | | |
| 48 | GDC_HPYL_{—} | 506250 | 507134 | 294 | + | site-specific DNA- |
| | 506250 | | | | | methyltransferase |
| 49 | GDC_HPYL_{—} | 583607 | 583876 | 89 | + | probable DNA helicase |
| | 583607 | | | | | |
| 50 | GDC_HPYL_{—} | 583883 | 584437 | 184 | + | probable DNA helicase |
| | 583883 | | | | | |
| 51 | GDC_HPYL_{—} | 665045 | 665695 | 216 | + | putative lipopolysaccharide |
| | 665045 | | | | | biosynthesis protein |
| 52 | GDC_HPYL_{—} | 953783 | 954664 | 293 | − | acetate kinase |
| | 953783 | | | | | |
| 53 | GDC_HPYL_{—} | 954679 | 954900 | 73 | − | phosphate acetyltransferase |
| | 954679 | | | | | |
| 54 | GDC_HPYL_{—} | 954846 | 955217 | 123 | − | PHOSPHOTRANSACETYLASE |
| | 954846 | | | | | |
| 55 | GDC_HPYL_{—} | 955261 | 955557 | 98 | − | phosphate acetyltransferase |
| | 955261 | | | | | |
| 56 | GDC_HPYL_{—} | 1068602 | 1069459 | 285 | − | IS606 TRANSPOSASE |
| | 1068602 | | | | | |
| 57 | GDC_HPYL_{—} | 1069456 | 1069929 | 157 | − | transposase-like protein, |
| | 1069456 | | | | | PS3IS |
| 58 | GDC_HPYL_{—} | 1376803 | 1377126 | 107 | + | ribosomal protein |
| | 1376803 | | | | | |
| 59 | GDC_HPYL_{—} | 1474291 | 1474509 | 72 | + | Cell wall-associated hydrolase |
| | 1474291 | | | | | |
| 60 | GDC_HPYL_{—} | 1600102 | 1600689 | 195 | − | TYPE III DNA |
| | 1600102 | | | | | MODIFICATION ENZYME |
| 61 | GDC_MTUB_{—} | 26830 | 27534 | 234 | − | putative protoporphyrinogen |
| | 26830 | | | | | oxidase |
| 62 | GDC_MTUB_{—} | 36276 | 36785 | 169 | − | fibronectin-attachment protein |
| | 36276 | | | | | FAP-P |
| 63 | GDC_MTUB_{—} | 76032 | 76595 | 187 | + | retinoblastoma inhibiting gene |
| | 76032 | | | | | 1 |
| 64 | GDC_MTUB_{—} | 80423 | 81214 | 263 | − | mucin 5 |
| | 80423 | | | | | |
| 65 | GDC_MTUB_{—} | 167239 | 168084 | 281 | + | putative secreted peptidase |
| | 167239 | | | | | |
| 66 | GDC_MTUB_{—} | 214625 | 215116 | 163 | − | glycoprotein gp2 |
| | 214625 | | | | | |
| 67 | GDC_MTUB_{—} | 424142 | 424657 | 171 | − | PPE FAMILY PROTEIN |
| | 424142 | | | | | |
| 68 | GDC_MTUB_{—} | 459316 | 461076 | 586 | + | 63 kDa protein |
| | 459316 | | | | | |
| 69 | GDC_MTUB_{—} | 549643 | 550758 | 371 | − | carR |
| | 549643 | | | | | |
| 70 | GDC_MTUB_{—} | 566823 | 567284 | 153 | + | MAPK-interacting and |
| | 566823 | | | | | spindle-stabilizing protein |
| 71 | GDC_MTUB_{—} | 591109 | 591345 | 78 | + | excisionase, putative |
| | 591109 | | | | | |
| 72 | GDC_MTUB_{—} | 663028 | 663426 | 132 | + | PROBABLE |
| | 663028 | | | | | RIBONUCLEOSIDE- |
| | | | | | | DIPHOSPHATE |
| | | | | | | REDUCTASE |
| 73 | GDC_MTUB_{—} | 688806 | 689060 | 84 | + | MCE-FAMILY PROTEIN |
| | 688806 | | | | | MCE2B |
| 74 | GDC_MTUB_{—} | 701762 | 702643 | 293 | − | u1764ad |
| | 701762 | | | | | |
| 75 | GDC_MTUB_{—} | 731710 | 731877 | 55 | + | ribosomal protein L33 |
| | 731710 | | | | | |
| 76 | GDC_MTUB_{—} | 772761 | 773402 | 213 | − | ENSANGP00000004917 |
| | 772761 | | | | | |
| 77 | GDC_MTUB_{—} | 868821 | 869216 | 131 | − | cold-shock induced protein of |
| | 868821 | | | | | the Srp1p/Tip1p |
| 78 | GDC_MTUB_{—} | 890358 | 891254 | 298 | − | orf2 |
| | 890358 | | | | | |
| 79 | GDC_MTUB_{—} | 904043 | 904840 | 265 | + | aminoimidazole ribotide |
| | 904043 | | | | | synthetase |
| 80 | GDC_MTUB_{—} | 1045383 | 1046129 | 248 | + | u650i |
| | 1045383 | | | | | |
| 81 | GDC_MTUB_{—} | 1068100 | 1068726 | 208 | − | anchorage subunit of a- |
| | 1068100 | | | | | agglutinin; Aga1p |
| 82 | GDC_MTUB_{—} | 1115707 | 1116369 | 220 | − | mucin 7 precursor, salivary |
| | 1115707 | | | | | |
| 83 | GDC_MTUB_{—} | 1124996 | 1125712 | 238 | − | putative oxidoreductase |
| | 1124996 | | | | | |
| 84 | GDC_MTUB_{—} | 1138949 | 1139665 | 238 | − | platelet binding protein GspB |
| | 1138949 | | | | | |
| 85 | GDC_MTUB_{—} | 1170285 | 1170749 | 154 | − | MC8 |
| | 1170285 | | | | | |
| 86 | GDC_MTUB_{—} | 1176592 | 1176858 | 88 | + | gp85 |
| | 1176592 | | | | | |
| 87 | GDC_MTUB_{—} | 1202653 | 1203198 | 181 | − | s19 chorion protein |
| | 1202653 | | | | | |
| 88 | GDC_MTUB_{—} | 1231843 | 1232460 | 205 | + | carboxylesterase |
| | 1231843 | | | | | |
| 89 | GDC_MTUB_{—} | 1241031 | 1241468 | 145 | − | PE |
| | 1241031 | | | | | |
| 90 | GDC_MTUB_{—} | 1252888 | 1253748 | 286 | − | ppg3 |
| | 1252888 | | | | | |
| 91 | GDC_MTUB_{—} | 1264312 | 1264554 | 80 | + | ketoacyl-CoA thiolase-related |
| | 1264312 | | | | | protein |
| 92 | GDC_MTUB_{—} | 1286282 | 1286587 | 101 | − | pterin-4-alpha-carbinolamine |
| | 1286282 | | | | | dehydratase |
| 93 | GDC_MTUB_{—} | 1301742 | 1302053 | 103 | − | similar to ORF starts at 87, |
| | 1301742 | | | | | first start codon |
| 94 | GDC_MTUB_{—} | 1351907 | 1352614 | 235 | − | ppg3 |
| | 1351907 | | | | | |
| 95 | GDC_MTUB_{—} | 1476279 | 1476647 | 122 | − | Cell wall-associated hydrolase |
| | 1476279 | | | | | |
| 96 | GDC_MTUB_{—} | 1485311 | 1486399 | 362 | − | 4-hydroxyphenylpyruvate |
| | 1485311 | | | | | dioxygenase C terminal |
| 97 | GDC_MTUB_{—} | 1486309 | 1487727 | 472 | − | cell wall surface anchor family |
| | 1486309 | | | | | protein |
| 98 | GDC_MTUB_{—} | 1515112 | 1515846 | 244 | − | putative ABC transporter ATP |
| | 1515112 | | | | | binding protein |
| 99 | GDC_MTUB_{—} | 1515464 | 1516198 | 244 | − | extracellular protein, gamma- |
| | 1515464 | | | | | D-glutamate-meso-d . . . |
| 100 | GDC_MTUB_{—} | 1596569 | 1596892 | 107 | − | putative translation initiation |
| | 1596569 | | | | | factor IF-2 |
| 101 | GDC_MTUB_{—} | 1600905 | 1601861 | 318 | − | carboxylesterase family |
| | 1600905 | | | | | protein |
| 102 | GDC_MTUB_{—} | 1616064 | 1616951 | 295 | − | PUTATIVE |
| | 1616064 | | | | | TRANSCRIPTION |
| | | | | | | REGULATOR PROTEIN |
| 103 | GDC_MTUB_{—} | 1672449 | 1673216 | 255 | + | MAV278 |
| | 1672449 | | | | | |
| 104 | GDC_MTUB_{—} | 1673708 | 1675000 | 430 | − | MAV301 |
| | 1673708 | | | | | |
| 105 | GDC_MTUB_{—} | 1699549 | 1700226 | 225 | + | gmdA |
| | 1699549 | | | | | |
| 106 | GDC_MTUB_{—} | 1742061 | 1742858 | 265 | − | ENSANGP00000020758 |
| | 1742061 | | | | | |
| 107 | GDC_MTUB_{—} | 1782153 | 1782932 | 259 | + | GLP_26_54603_52153 |
| | 1782153 | | | | | |
| 108 | GDC_MTUB_{—} | 2060659 | 2061114 | 151 | + | nuclear factor of kappa light |
| | 2060659 | | | | | polypeptide gene |
| 109 | GDC_MTUB_{—} | 2093062 | 2093994 | 310 | − | PROBABLE 6- |
| | 2093062 | | | | | PHOSPHOGLUCONATE |
| | | | | | | DEHYDROGENASE GND1 |
| 110 | GDC_MTUB_{—} | 2105797 | 2106912 | 371 | + | ATP-binding subunit of ABC- |
| | 2105797 | | | | | transport system |
| 111 | GDC_MTUB_{—} | 2133554 | 2134069 | 171 | − | KIAA0324 protein |
| | 2133554 | | | | | |
| 112 | GDC_MTUB_{—} | 2183418 | 2184026 | 202 | − | putative transport protein |
| | 2183418 | | | | | |
| 113 | GDC_MTUB_{—} | 2192571 | 2193488 | 305 | − | putative oxidoreductase |
| | 2192571 | | | | | |
| 114 | GDC_MTUB_{—} | 2234641 | 2234889 | 82 | − | DNA-binding protein, CopG |
| | 2234641 | | | | | family |
| 115 | GDC_MTUB_{—} | 2320829 | 2321062 | 77 | + | DNA-binding protein, CopG |
| | 2320829 | | | | | family |
| 116 | GDC_MTUB_{—} | 2321250 | 2322509 | 419 | − | cell wall surface anchor family |
| | 2321250 | | | | | protein |
| 117 | GDC_MTUB_{—} | 2487508 | 2488524 | 338 | − | ORF1 |
| | 2487508 | | | | | |
| 118 | GDC_MTUB_{—} | 2567990 | 2568457 | 155 | + | B1158F07.3 |
| | 2567990 | | | | | |
| 119 | GDC_MTUB_{—} | 2577106 | 2577699 | 197 | + | POSSIBLE CONSERVED |
| | 2577106 | | | | | MEMBRANE PROTEIN |
| 120 | GDC_MTUB_{—} | 2577486 | 2577920 | 144 | + | POSSIBLE CONSERVED |
| | 2577486 | | | | | MEMBRANE PROTEIN |
| 121 | GDC_MTUB_{—} | 2690012 | 2690509 | 165 | + | PROBABLE CONSERVED |
| | 2690012 | | | | | INTEGRAL MEMBRANE |
| | | | | | | PROTEIN |
| 122 | GDC_MTUB_{—} | 2698040 | 2698243 | 67 | − | POSSIBLE CONSERVED |
| | 2698040 | | | | | MEMBRANE PROTEIN |
| 123 | GDC_MTUB_{—} | 2712275 | 2714008 | 577 | + | MLCL536.10 protein |
| | 2712275 | | | | | |
| 124 | GDC_MTUB_{—} | 2725593 | 2725859 | 88 | − | PROBABLE HYDROGEN |
| | 2725593 | | | | | PEROXIDE-INDUCIBLE |
| | | | | | | GENES |
| 125 | GDC_MTUB_{—} | 2733212 | 2734420 | 402 | − | lycoprotein gp2 |
| | 2733212 | | | | | |
| 126 | GDC_MTUB_{—} | 2828257 | 2828937 | 226 | + | MC8 |
| | 2828257 | | | | | |
| 127 | GDC_MTUB_{—} | 2895354 | 2897222 | 622 | + | antigen T5 |
| | 2895354 | | | | | |
| 128 | GDC_MTUB_{—} | 2983047 | 2984033 | 328 | − | MC8 |
| | 2983047 | | | | | |
| 129 | GDC_MTUB_{—} | 3005316 | 3005696 | 126 | − | ABC transporter, ATP-binding |
| | 3005316 | | | | | protein |
| 130 | GDC_MTUB_{—} | 3048559 | 3049095 | 178 | − | recX protein |
| | 3048559 | | | | | |
| 131 | GDC_MTUB_{—} | 3065095 | 3066549 | 484 | + | ppg3 |
| | 3065095 | | | | | |
| 132 | GDC_MTUB_{—} | 3100192 | 3100452 | 86 | − | IS1537, transposase |
| | 3100192 | | | | | |
| 133 | GDC_MTUB_{—} | 3129118 | 3129594 | 158 | − | KIAA1139 protein |
| | 3129118 | | | | | |
| 134 | GDC_MTUB_{—} | 3237815 | 3238096 | 93 | − | acylphosphatase |
| | 3237815 | | | | | |
| 135 | GDC_MTUB_{—} | 3283182 | 3283718 | 178 | − | Putative mycocerosyl |
| | 3283182 | | | | | transferase in MAS 5′r . . . |
| 136 | GDC_MTUB_{—} | 3289702 | 3290232 | 176 | + | POSSIBLE TRANSPOSASE |
| | 3289702 | | | | | |
| 137 | GDC_MTUB_{—} | 3319076 | 3319546 | 156 | − | u0002d |
| | 3319076 | | | | | |
| 138 | GDC_MTUB_{—} | 3339006 | 3339851 | 281 | − | membrane glycoprotein |
| | 3339006 | | | | | |
| 139 | GDC_MTUB_{—} | 3356995 | 3357831 | 278 | − | sensor histidine kinase |
| | 3356995 | | | | | |
| 140 | GDC_MTUB_{—} | 3381198 | 3381755 | 185 | + | MC8 |
| | 3381198 | | | | | |
| 141 | GDC_MTUB_{—} | 3388071 | 3389003 | 310 | + | cellulosomal scaffoldin |
| | 3388071 | | | | | anchoring protein C |
| 142 | GDC_MTUB_{—} | 3482312 | 3482770 | 152 | − | MC8 |
| | 3482312 | | | | | |
| 143 | GDC_MTUB_{—} | 3581973 | 3582620 | 215 | + | similar to mucin, submaxillary - |
| | 3581973 | | | | | pig |
| 144 | GDC_MTUB_{—} | 3711717 | 3712613 | 298 | − | orf2 |
| | 3711717 | | | | | |
| 145 | GDC_MTUB_{—} | 3716987 | 3718534 | 515 | − | similar to profilaggrin - human |
| | 3716987 | | | | | (fragments) |
| 146 | GDC_MTUB_{—} | 3754581 | 3755711 | 376 | − | putative transposase |
| | 3754581 | | | | | |
| 147 | GDC_MTUB_{—} | 3794808 | 3795026 | 72 | − | deoxyxylulose-5-phosphate |
| | 3794808 | | | | | synthase |
| 148 | GDC_MTUB_{—} | 3796793 | 3797512 | 239 | + | membrane glycoprotein |
| | 3796793 | | | | | [imported] - equine |
| | | | | | | herpesvirus |
| 149 | GDC_MTUB_{—} | 3879013 | 3879534 | 173 | − | ribosomal protein S11 |
| | 3879013 | | | | | |
| 150 | GDC_MTUB_{—} | 3921024 | 3921665 | 213 | − | 3-oxoacyl-(acyl-carrier- |
| | 3921024 | | | | | protein) reductase |
| 151 | GDC_MTUB_{—} | 3974481 | 3975056 | 191 | + | mucin 10 |
| | 3974481 | | | | | |
| 152 | GDC_MTUB_{—} | 3994808 | 3995446 | 212 | + | MAV278 |
| | 3994808 | | | | | |
| 153 | GDC_MTUB_{—} | 3998938 | 3999642 | 234 | − | protease inhibitor/seed |
| | 3998938 | | | | | storage/lipid transfer |
| 154 | GDC_MTUB_{—} | 4021183 | 4021425 | 80 | − | PUTATIVE TRNA/RRNA |
| | 4021183 | | | | | METHYLTRANSFERASE |
| 155 | GDC_MTUB_{—} | 4045946 | 4046290 | 114 | − | chalcone/stilbene synthase |
| | 4045946 | | | | | family protein |
| 156 | GDC_MTUB_{—} | 4053033 | 4053635 | 200 | + | putative protein (2G313) |
| | 4053033 | | | | | |
| 157 | GDC_MTUB_{—} | 4140236 | 4140460 | 74 | − | DNA-binding protein, CopG |
| | 4140236 | | | | | family |
| 158 | GDC_MTUB_{—} | 4169350 | 4169706 | 118 | + | PROBABLE CUTINASE |
| | 4169350 | | | | | PRECURSOR CUT5 |
| 159 | GDC_MTUB_{—} | 4170798 | 4171211 | 137 | + | PUTATIVE |
| | 4170798 | | | | | OXIDOREDUCTASE |
| 160 | GDC_MTUB_{—} | 4252190 | 4252921 | 243 | + | Salivary gland secretion 1 |
| | 4252190 | | | | | CG3047-PA |
| 161 | GDC_MTUB_{—} | 4260620 | 4261213 | 197 | + | SPAPB15E9.01c |
| | 4260620 | | | | | |
| 162 | GDC_MTUB_{—} | 4302166 | 4302858 | 230 | + | u1764ad |
| | 4302166 | | | | | |
| 163 | GDC_MTUB_{—} | 4317863 | 4318309 | 148 | + | POSSIBLE TRANSPOSASE |
| | 4317863 | | | | | [SECOND PART] |
| 164 | GDC_MTUB_{—} | 4341852 | 4342388 | 178 | − | GLP_49_64409_65443 |
| | 4341852 | | | | | |
| 165 | GDC_MTUB_{—} | 4391527 | 4391988 | 153 | − | AT9S |
| | 4391527 | | | | | |
| 166 | gi!Sars174_ref | 701 | 1225 | 174 | + | ABC transporter ATP binding |
| | seq_OUTPUT | | | | | protein/Cytochrome c oxidase |
| | F_GDC_701_{—} | | | | | folding protein |
| | 1225 | | | | | |
| 167 | gi!Sars68_refs | 1397 | 1603 | 68 | + | Major facilitator for |
| | eq_OUTPUTF_{—} | | | | | superfamily protein or |
| | GDC_1397_{—} | | | | | serine/threonine kinase 2 |
| | 1603 | | | | | |
| 168 | gi!Sars61_refs | 8828 | 9013 | 61 | + | Putative protein |
| | eq_OUTPUTF_{—} | | | | | |
| | GDC_8828_{—} | | | | | |
| | 9013 | | | | | |
| 169 | gi!Sars78_refs | 24492 | 24764 | 90 | + | NADH dehydrogenase I chain |
| | eq_OUTPUTF_{—} | | | | | |
| | GDC_28559_{—} | | | | | |
| | 28795 | | | | | |

A systematic sensitivity and specificity analysis of GeneDecipher has been done on 10 microbial genomes (FIG. 3). Further analysis of GeneDecipher on viral genomes is presented here.

SARS-CoV genome sequence:Sequences of the 18 SARS-CoV strains available in the GenBank database (http://www.ncbi.nlm.nih.gov/Entrez/genomes/viruses) were downloaded and analyzed. These include SARS-CoV Refseq (NC_{—}004718.3), SARS-CoV TWC(AY32118), SIN2774(AY283798), SIN2748(AY283797) SIN267\hat{ }(AY283796), SIN2677(AY283794), SIN25ti6(AY283794), Frankfurt 1 (AY291315), BJ04(AY279354) BJ03(AY278490), BJ02(AY278487), GZ01(AY278848), CUHKW1(AY278554), TOR2(AY274119), TW1(AY291451), BJ01(AY278488), Urban(AY278741), HKU-39849(AY278491). Other information related to protein coding genes was retrieved from http://www.ncbi.nlm.nih.gov/genomes/SARS/SAks.html.

Testing of GeneDecipher on Viral Genomes:

To test our method on viral genomes the applicants first analyzed Human Respiratory Syncytial Virus (HRSV), complete genome using GeneDecipher. Comparison of GeneDecipher results with state of the art method ZCURVE_CoV has been done (Table 3). ZCURVE_CoV is able to predict 8 annotated proteins out of 11 reported at NCBI without any false positives. ZCURVE_CoV was unable to predict the following three genes: PID 9629200 (location 626 . . . 1000, non-structural protein2 (NS2)); PID 9629205 (location 4690 . . . 5589, attachment glycoprotein (G)); and PID 9629208 (location 8171 . . . 8443, matrix protein 2(M2)). GeneDecipher predicted 10 out of total 11 annotated proteins of HRSV without any false positives. The gene missed by GeneDecipher was PID 9629208 (location 8171 . . . 8443, matrix protein 2) which was notably missed by ZCURVE_CoV too.

This successful prediction of protein coding regions in HRSV genome increases our confidence to predict protein coding regions on newly sequenced SARS-CoV genomes.

Analysis of SARS-CoV Using GeneDecipher:

The applicants analyzed all 18 strains of SARS-CoV using GeneDecipher. (Detailed results are available on the website given above). GeneDecipher predicts a total of 15 protein coding regions in SARS-CoV genomes including both the polyproteins 1a, 1ab (Sars2628 C-terminal end of Polyprotein 1ab), and all four known structural proteins (M, N, S, and E) for each of the 18 strains. GeneDecipher also predicts 6 to 8 additional coding regions depending on the genome sequence of the strain used. The length of these additional coding regions varied between 61 and 274 amino acids.

GeneDecipher predicts 12 coding regions which are common to all 18 strains (Table 4), and one coding region (Sars63, sars6 at NCBI refseq genome) present in 5 strains. GeneDecipher predicts gene Sars90 in GZ01 strain, and Sars154 (Sars 3b at NCBI refseq genome) in BJ02 strain specifically.

These 12 common protein coding regions consist of the 6 basic proteins of SARS-CoV (2 polyproteins and the 4 structural proteins); Sars274 (Sars3a at NCBI refseq database), Sars122 (Sars7a at NCBI refseq database), Sars78 (already reported with start shifted as ORF14/Sars9c in TOR2 strain); and three newly predicted (false positives with respect to current annotation at NCBI) protein coding regions Sars 174, Sars68, and Sars61. The three newly predicted genes lie completely within polyprotein 1a genomic region. Although our method discards such genes in bacterial genomes, possibility of finding such genes in viral genomes has not been ruled out. As these genes are present in all 18 strains it is likely that they are protein coding genes.

The applicants predict three more coding regions Sars63, Sars154, and Sars90 apart from the 12 discussed above. Sars63 is identified in 5 strains and not identified in remaining 13 strains. This coding region is already reported in NCBI refseq (Sars6). Here the applicants can not comment much about the existence of Sars63 (Sars6 at NCBI refseq) because it is identified in 5 strains and not identified in rest 13. This is due to high density of non-synonymous mutations across strains in this region. Two coding regions Sars154 (sars3b at NCBI), and Sars90 (newly predicted in GZ01 starin) are identified in only one strain. Since these two coding regions are identified in only one strain, they are less likely to be protein coding regions, as also suggested by ZCURVE_CoV (Chen et al., 2003) analysis. The locations of these three genes in different strains are provided in Table 5.

Since the peptide libraries are made from the genome sequences of various organisms, the evolutionary origin of a given protein can be traced. If the protein is rich in heptapeptides found occurring in viral genomes then that protein is considered to be of viral origin. The applicants found that 5 core proteins (two polyproteins and three structural proteins M, N, and S) are of viral origin. The remaining, including 3 new predictions, are of prokaryotic origin. It is interesting to that from the same DNA region the applicants are getting proteins in different frames which contain peptides from different origin. Here, how same DNA sequence can code for both bacterial and viral origin is intriguing. This might explain why these new protein coding genes were not detected in primary attempts based on homology to other known viral genome sequences.

Comparison with the Existing System—ZCURVE_CoV.

Comparison of GeneDecipher, ZCURVE_CoV results with the known annotations for Urbani and TOR2 strains of SARS-CoV are presented in Tables 6a and 6b.

In general, GeneDecipher results are in good agreement with the known annotations. In case of Urbani strain GeneDecipher predicts all the known genes except Sars84(X5), Sars63(X3) and Sars154(X2). Sars84(X5) and Sars63(X3) are supported by ZCURVE_CoV whereas Sars154(X2) is missed by both the methods. GeneDecipher predicts four new genes in this strain which incidentally are not supported by ZCURVE_CoV. It is noticeable that out of these four genes Sars78 is already known for strain TOR2 as ORF14/Sars9c. This supports the likelihood of the gene being present in Urbani strain. However, ZCURVE_CoV predicts 2 new genes which are not supported by GeneDecipher either.

GeneDecipher predictions for TOR2 strain are identical with those for Urbani strain. In this strain GeneDecipher predicts 9 known genes but fails to predict 6 genes with known annotations. These 6 genes are: Sars154 (ORF4), Sars98 (ORF13), Sars63 (ORF7), Sars44 (ORF9), Sars39 (ORF10), and Sars84 (ORF11). Of these, Sars154 (ORF4) and Sars98 (ORF13) are also missed by ZCURVE_CoV. It is to be noted that both Sars44 (ORF9) and Sars39 (ORF10) are ORFs very small in length (44 and 39 amino acids respectively), and their presence too is not consistent across various SARS strains. Sars63 (ORF7) has been predicted by GeneDecipher in 5 other strains but not in the two strains considered here.

Mutation Analysis:

Analysis using multiple sequence alignment (ClustalW) for 3 newly predicted protein coding genes Sars174, Sars68 and Sars61 across all 18 strains shows:
1. Sars68 has one point mutation at location 80 GAT->GGT (D->G) SIN2677 strain.
2. Sars174 has two synonymous point mutations at location 204 CGA->CGC in GZ01 strain and at location 447 CTG->CTT in BJ04 strain.
3. Sars61 has one point mutation at location 119 CTG->CAG (L->Q) in GZ01 strain.

These three newly predicted genes are present in all 18 strains without significant mutations and has no significant hits with BLASTP in non-redundant database. This indicates that these three proteins might have crucial biological functions specific to SARS-CoV. Therefore these coding sequences might serve as candidate drug targets against SARS.

Function Assignment:

In total the applicants predict 15 coding regions in SARS-CoV out of which functions of the four structural proteins (M, N, S and E) have already been assigned. Although the polyprotein 1ab has been assigned only replicase activity, our analysis implies that the replicase activity is associated with Sars2628 (C terminal of ORF 1ab) fragment. The complete 1ab polyprotein contains 6 functional signatures of which polyprotein 1a contains signatures associated with metabolic enzymes (Table 7a). Functions were assigned to the polyproteins on the basis of peptides (length 7 or more amino acids) occurring in proteins having similar functions in at least 5 different organisms. Other predicted genes/protein coding regions contain peptides which occur in fewer genomes. Based on these peptides the applicants suggest functions, albeit with lesser confidence (Table 7b). The biological relevance of these finding remains to be explored.

TABLE 3
Comparison of GeneDecipher results with ZCURVE_CoV results
on HRSV genome, with respect to annotated genes

| Annotated genes | | | ZCURVE_CoV | | | GeneDecipher | | |
|---|---|---|---|---|---|---|---|---|
| Start | End | Length | Start | End | Length | Start | End | Length |
| 99 | 518 | 139 | 99 | 518 | 139 | 99 | 518 | 139 |
| 626 | 1000 | 124 | — | — | — | 626 | 1000 | 124 |
| 1140 | 2315 | 391 | 1140 | 2315 | 391 | 1140 | 2315 | 391 |
| 2348 | 3073 | 241 | 2348 | 3073 | 241 | 2348 | 3073 | 241 |
| 3263 | 4033 | 256 | 3158 | 4033 | 291 | 3158 | 4033 | 291 |
| 4303 | 4500 | 65 | 4303 | 4500 | 65 | 4303 | 4500 | 65 |
| 4690 | 5589 | 299 | — | — | — | 4690 | 5589 | 299 |
| 5666 | 7390 | 574 | 5666 | 7390 | 574 | 5621 | 7390 | 589 |
| 7618 | 8205 | 195 | 7618 | 8205 | 195 | 7618 | 8205 | 195 |
| 8171 | 8443 | 90 | — | — | — | — | — | — |
| 8509 | 15009 | 2166 | 8443 | 15009 | 2188 | 8443 | 15009 | 2188 |

TABLE 4
Protein coding genes predicted by GeneDecipher
in SARS-CoV Refseq common to all 18 strains.

| S. | | | | Length | | |
|---|---|---|---|---|---|---|
| No. | Start | Stop | Frame | bp | aa | Feature |
| 1 | 265 | 13413 | 1+ | 13149 | 4382 | Sars1a polyprotein |
| 2 | 701 | 1225 | 2+ | 525 | 174 | Sars174(new predic- |
| | | | | | | tion) |
| 3 | 1397 | 1603 | 2+ | 207 | 68 | Sars68(new predic- |
| | | | | | | tion) |
| 4 | 8828 | 9013 | 2+ | 186 | 61 | Sars61(new predic- |
| | | | | | | tion) |
| 5 | 13599 | 21485 | 3+ | 7887 | 2628 | Sars2628(C-terminal |
| | | | | | | end of polyprotein |
| | | | | | | lab) |
| 6 | 21492 | 25259 | 3+ | 3768 | 1255 | Spike (S) protein |
| 7 | 25268 | 26092 | 2+ | 825 | 274 | Sars274(Sars 3a) |
| 8 | 26117 | 26347 | 2+ | 231 | 76 | Sars76(Sars4) |
| 9 | 26398 | 27063 | 1+ | 666 | 221 | Sars221(Sars5) |
| 10 | 27273 | 27641 | 3+ | 369 | 122 | Sars122(Sars7a) |
| 11 | 28120 | 29388 | 1+ | 1269 | 422 | Sars422(Sars9a) |
| 12 | 28559 | 28795 | 2+ | 237 | 78 | Sars78 (Identical |
| | | | | | | to ORF 14/Sars9c |
| | | | | | | in TOR2 with |
| | | | | | | shifted start) |

TABLE 5
Identification of Sars90, Sars63, Sars154 as protein coding
genes by GeneDecipher in various strains of SARS-CoV

| S. | Strain | Sars90 (New | Sars63(Sars6 | Sars154(Sars |
|---|---|---|---|---|
| No. | name | prediction) | at NCBI) | 3b at NCBI) |
| 1 | SIN2748 | — | — | — |
| 2 | BJ01 | — | 27055 . . . 27246 | — |
| 3 | BJ02 | — | 27074 . . . 27265 | 25689 . . . |
| | | | | 26153 |
| 4 | BJ03 | — | 27070 . . . 27261 | — |
| 5 | BJ04 | — | 27058 . . . 27249 | — |
| 6 | Frank- | — | — | — |
| | furtt1 | | | |
| 7 | Urbani | — | — | — |
| 8 | GZ01 | 24492 . . . 24764 | 27058 . . . 27249 | — |
| 9 | SIN2500 | — | — | — |
| 10 | SIN2677 | — | — | — |
| 11 | SIN2679 | — | — | — |
| 12 | SIN2774 | — | — | — |
| 13 | CHUKW1 | — | — | — |
| 14 | TW1 | — | — | — |
| 15 | TWC | — | — | — |
| 16 | HKU- | — | — | — |
| | 39849 | | | |
| 17 | Refseq | — | — | — |
| 18 | TOR2 | — | — | — |

TABLE 6(a)
Comparison of GeneDecipher results with ZCURVE_CoV results on
SARS-CoV genome Urbani strain, with respect to annotated genes

| Annotated genes | | | ZCURVE_CoV | | | GeneDecipher | | | |
|---|---|---|---|---|---|---|---|---|---|
| Start | End | Length | Start | End | Length | Start | End | Length | Features |
| 265 | 13398 | 4377 | 265 | 13398 | 4377 | 265 | 13413 | 4382 | ORF 1a |
| — | — | — | — | — | — | 701 | 1225 | 174 | Sars174(New |
| | | | | | | | | | prediction by |
| | | | | | | | | | GeneDecipher) |
| — | — | — | — | — | — | 1397 | 1603 | 68 | Sars68(New |
| | | | | | | | | | prediction by |
| | | | | | | | | | GeneDecipher) |
| — | — | — | — | — | — | 8828 | 9013 | 61 | Sars61(New |
| | | | | | | | | | prediction by |
| | | | | | | | | | GeneDecipher) |
| 13398 | 21485 | 2695 | 13398 | 21485 | 2695 | 13599 | 21485 | 2628 | ORF 1b |
| 21492 | 25259 | 1255 | 21492 | 25259 | 1255 | 21492 | 25259 | 1255 | S protein |
| 25268 | 26092 | 274 | 25268 | 26092 | 274 | 25268 | 26092 | 274 | Sars274(X1) |
| 25689 | 26153 | 154 | — | — | — | — | — | — | Sars154(X2) |
| 26117 | 26347 | 76 | 26117 | 26347 | 76 | 26117 | 26347 | 76 | E protein |
| 26398 | 27063 | 221 | 26398 | 27063 | 221 | 26389 | 27063 | 224 | M protein |
| 27074 | 27265 | 63 | 27074 | 27265 | 63 | — | — | — | Sars63(X3) |
| 27273 | 27641 | 122 | 27273 | 27641 | 122 | 27273 | 27641 | 122 | Sars122(X4) |
| — | — | — | 27638 | 27772 | 44 | — | — | — | Sars44 |
| — | — | — | 27779 | 27898 | 39 | — | — | — | Sars39 |
| 27864 | 28118 | 84 | 27864 | 28118 | 84 | — | — | — | Sars84(X5) |
| 28120 | 29388 | 422 | 28120 | 29388 | 422 | 28120 | 29388 | 422 | N protein |
| — | — | — | — | — | — | 28559 | 28795 | 78 | Sars78(Identical |
| | | | | | | | | | to ORF |
| | | | | | | | | | 14/Sars9c in |
| | | | | | | | | | TOR2 with |
| | | | | | | | | | shifted start) |

TABLE 6(b)
Comparison of GeneDecipher results with ZCURVE_CoV results on
SARS-CoV genome TOR2 strain, with respect to annotated genes

| | | | ZCURVE_CoV | | | GeneDecipher | | | |
|---|---|---|---|---|---|---|---|---|---|
| Annotated genes | | | predicted genes | | | predicted genes | | | |
| Start | End | Length | Start | End | Length | Start | End | Length | Features |
| 265 | 13398 | 4377 | 265 | 13398 | 4377 | 265 | 13413 | 4382 | ORF 1a |
| — | — | — | — | — | — | 701 | 1225 | 174 | Sars174(New |
| | | | | | | | | | prediction by |
| | | | | | | | | | GeneDecipher) |
| — | — | — | — | — | — | 1397 | 1603 | 68 | Sars68(New |
| | | | | | | | | | prediction by |
| | | | | | | | | | GeneDecipher) |
| — | — | — | — | — | — | 8828 | 9013 | 61 | Sars61(New |
| | | | | | | | | | prediction by |
| | | | | | | | | | GeneDecipher) |
| 13398 | 21485 | 2695 | 13398 | 21485 | 2695 | 13599 | 21485 | 2628 | ORF 1b |
| 21492 | 25259 | 1255 | 21492 | 25259 | 1255 | 21492 | 25259 | 1255 | S protein |
| 25268 | 26092 | 274 | 25268 | 26092 | 274 | 25268 | 26092 | 274 | ORF3(Sars274) |
| 25689 | 26153 | 154 | — | — | — | — | — | — | ORF4(Sars154) |
| 26117 | 26347 | 76 | 26117 | 26347 | 76 | 26117 | 26347 | 76 | E protein |
| 26398 | 27063 | 221 | 26398 | 27063 | 221 | 26389 | 27063 | 224 | M protein |
| 27074 | 27265 | 63 | 27074 | 27265 | 63 | — | — | — | Sars63(ORF7) |
| 27273 | 27641 | 122 | 27273 | 27641 | 122 | 27273 | 27641 | 122 | Sars122(ORF8) |
| 27638 | 27772 | 44 | 27638 | 27772 | 44 | — | — | — | Sars44(ORF9) |
| 27779 | 27898 | 39 | 27779 | 27898 | 39 | — | — | — | Sars39(ORF10) |
| 27864 | 28118 | 84 | 27864 | 28118 | 84 | — | — | — | Sars84(ORF11) |
| 28120 | 29388 | 422 | 28120 | 29388 | 422 | 28120 | 29388 | 422 | N protein |
| 28130 | 28426 | 98 | — | — | — | — | — | — | ORF13 |
| 28583 | 28795 | 70 | — | — | — | 28559 | 28795 | 78 | Sars78(Identical |
| | | | | | | | | | to ORF |
| | | | | | | | | | 14/Sars9c in |
| | | | | | | | | | TOR2 with |
| | | | | | | | | | shifted start) |

TABLE 7(a)
Functional assignment of polyproteins
in SARS (Urbani) Genome using PLHOST

| S. | NCBI | Conserved peptide | |
|---|---|---|---|
| No. | annotation | signature | Function assigned |
| 1 | Sars1ab | RIRASLPT | Phosphoglycerate kinase |
| | (Poly | | |
| | protein1ab) | | |
| | | RSETLLPL | Sulfite reductase (NADPH), |
| | | | Flavoprotein |
| | | | beta subunit |
| | | LDKLKSLL | Probable acyl-CoA thiolase |
| | | ATVVIGTS | cell division protein ftsZ |
| | | NVAITRAK | DNA-binding protein, |
| | | | probably DNA |
| | | | helicase |
| | | LQGPPGTGK | DNA helicase related |
| | | | protein |
| 2 | Sars1a poly | RIRASLPT | Phosphoglycerate kinase |
| | protein1a | | |
| | | RSETLLPL | Sulfite reductase (NADPH), |
| | | | Flavoprotein |
| | | | beta subunit |
| | | LDKLKSLL | Probable acyl-CoA thiolase |
| 3 | Sars 2628 | ATVVIGTS | cell division protein ftsZ |
| | (C terminal | | |
| | of Sars1ab) | | |
| | | NVAITRAK | DNA-binding protein, |
| | | | probably DNA |
| | | | helicase |
| | | LQGPPGTGK | DNA helicase related |
| | | | protein |

TABLE 7(b)
Suggested functions for some of the non-structural
genes in SARS-CoV using PLHOST

| S. | | Peptide | |
|---|---|---|---|
| No. | Gene | Signature | Suggested function |
| 1 | Sars174(new | TLSKGNAQ | ABC transporter ATP |
| | prediction) | | binding protein |
| | | | [Lactococcus lactis subsp. |
| | | | lactis] |
| | | VAQMGTLL | Cytochrome c oxidase |
| | | | folding protein |
| | | | [Synechocystis sp. |
| | | | PCC 6803] |
| 2 | Sars68(new | LVLVLILA | putative major facilitator |
| | prediction) | | superfamily protein |
| | | | [Schizosaccharomyces |
| | | | pombe] |
| | | TQTLKLDS | serine/threonine kinase 2; |
| | | | Serine/threonine |
| | | | protein kinase-2 |
| | | | [Homo sapiens] |
| 3* | Sars90(new | GLLHRGT | NADH Dehydrogenase I |
| | prediction | | Chain |
| | only in | | |
| | GZ01 strain) | | |
| 4 | Sars61(new | LLPLLAFL | Putative protein |
| | prediction) | | (Conserved across 2 |
| | | | organisms) |
| 5 | Sars274(Sars3a) | LLLFVTIY | Polyamine transport |
| | | | protein; Tpo1p |
| | | | [Saccharomyces |
| | | | cerevisiae] |
| 6 | Sars154(Sars3b) | QTLVLKML | K550.3.p [Caenorhabditis |
| | | | elegans] |
| 7 | Sars63(Sars6) | DDEELMEL | Elongation factor Tu |
| | | | [Lactococcus lactis |
| | | | subsp. lactis] |
| 8 | Sars122(Sars7a) | LIVAALVF | Putative transport |
| | | | transmembrane protein |
| | | | [Sinorhizobium meliloti] |
| | | RARSVSPK | Src homology domain 3 |
| | | | [Caenorhabditis |
| | | | elegans] |
| 9* | Sars78(Sars9c) | QLLAAVG | Gamma-glutamate kinase |
| | | | (Conserved across |
| | | | 8 organisms) |

*No conserved octapeptide was found. However, function has been assigned on the basis of the highly conserved heptapeptide.

From the aforementioned The applicants have disclosed 4 new genes including Sars78 in SARS-CoV. The analysis further corroborates the finding of ZCURVE_CoV (Chen et al., 2003) that ORF Sars154 (listed in Refseq as Sars3b) is unlikely to be a coding region. The applicants have also assigned functions to the two polyproteins 1ab and 1a. In addition to replication associated function of C-terminal of 1ab polyprotein, the applicants' analysis implies that the polyprotein 1a may be associated with metabolic enzyme like functions. In all, six peptide signatures are present in polyprotein 1ab. The applicants have suggested putative function for other 9 proteins including ones newly predicted Ly GeneDecipher.

Advantages:

1. Main advantage of the present invention is to provide a new method for prediction of protein coding DNA sequences without using any external evidences like ribosome binding sites, promoter sequences, transcription start sites or codon usage biases.
2. It provides a method for statistical analysis of protein coding DNA sequences that utilizes the biological information retained in the conserved peptides which withstood evolutionary pressure.
3. It provides a simple method for start site prediction of a protein coding gene.
4. It provides a method to detect organism specific, strain specific protein coding DNA sequences.
5. It provides novel protein coding DNA sequences, which could be used as potential drug targets.

### REFERENCES

Altschul, S. F., Gish, W., Miller, W., Myers, E. W., Lipman, D. J. (1990) Basic local alignment search tool. J. Mol. Biol., 215, 403-10
Bird, A. (1987) C_{P}G islands as gene markers in the vertebrate nucleus. Trends Genet., 3, 342-47
Chen, L., Ou, H., Zhang, R. and Zhang, C. (2003) ZCURVE_CoV: a new system to recognize protein coding genes in coronavirus, and its applications in analyzing SARS-CoV genomes. Biochemical and Biophysical Research Communications, 307, 382-8.
Delcher, A. L., Harmon, D., Kasif, S., White, O. and Salzberg, S. L. (1999) Improved microbial gene identification with GLIMMER. Nucleic Acid Research, 27, 4636-41.
Kehoe, M. A., et al., (1996) Horizontal gene transfer among group A streptococci: implications for pathogenesis and epidemiology. Trends Microbial., 4, 436-43.
Lukashin, A. V. and Borodovsky, M. (1998) GeneMark.hmm: New solution for gene finding. Nucleic Acid Research, 26, 1107-15.
Mathe, C., Sagot, M. F., Schiex, T. and Rouze, P. (2002) Current Methods of gene prediction their strength and the applicantsaknesses. Nucleic Acid Research, 30, 4103-17
Medigue, C., et al. (1999) Detecting and Analyzing DNA Sequencing Errors:Toward a Higher Quality of the Bacillus subtilis Genome Sequence. Genome Research, 9, 1116-27
Pearson, W. R. (1995) Comparison of methods for searching protein sequence databases. Protein Science, 4, 1145-60.
Salzberg, S. L., Delcher, A. L., Kasif, S. and White, O. (1998) Microbial gene identification using interpolated Markov models. Nucleic Acid Research, 26, 544-8.
Shibuya, T. and Rigoutsos, I. (2002) Dictionary-driven prokaryotic gene finding. Nucleic Acid Research, 30, 2710-25.
Brahmachari, S. K. and Dash, D. (2001) a computer based method for identifying peptides useful as drug targets. PCT international patent publication (WO 01/74130 A2, 11 Oct. 2001). Cumulative number of reported cases of severe acute respiratory syndrome (SARS) Geneva: World Health Organization, 2003. (Accessed Apr. 9, 2003 at http://www.who.int/csr/sarscountry/2003_{—}04_{—}04/en/.)
Drosten, C., Giinther, S. and Preiser, W., (2003) Identification of a Novel Coronavirus in Patients with Severe Acute Respiratory Syndrome. N Engl J. Med., (www.nejm.org on Apr. 10, 2003.)
Ksiazek, T. G., Dean Erdman, P. H. and Goldsmith, C. S. (2003) A Novel Coronavirus Associated with Severe Acute Respiratory Syndrome. N Engl J Med, 348, 1947-58.
Marra, M. A., Jones, S. J., Astell, C. R., Holt, R. A., Brooks-Wilson, A. (2003) The Genome sequence of the SARS-associated coronavirus. Science, 300, 1399-404.
Tsang, K. W., Ho, P. L. and Ooi, G. C., (2003) A cluster of cases of severe acute respiratory syndrome in Hong Kong. N Engl J Med, 348, 1977-85.

1. A computer based versatile method for identifying protein coding DNA sequences useful as drug targets said method comprising steps of:
a. generating peptide libraries from the known genomes with oligopeptide of length ‘N’ computationally arranged in an alphabetical order,
b. artificially translating the test genome to obtain a polypeptide in each reading frame,
c. converting each polypeptide sequence into an alphanumeric sequence with one corresponding to each reading frame on the basis of occurrence of these oligopeptides in the peptide libraries,
d. training Artificial Neural Network (ANN) with sigmoidal learning function to the alphanumeric sequences corresponding to known protein coding DNA sequences and known non-coding regions,
e. deciphering the protein coding regions in the test genome, and
f. identifying longer stretches of peptides mapped to large number of known genes serving as functional signatures.

2. A method claimed in claim 1 wherein the artificial neural network has one or more input layer, one or more hidden layer with varying number of neurons, and one or more output layer.

3. A method claimed in claim 1 wherein the number of neurons in the hidden layer is preferably 30.

4. A method claimed in claim 1 wherein the value of the ‘N’ is 4 or more.

5. A method claimed in claim 1 wherein the sigmoidal learning function has five parameters comprising total score, mean, fraction of zeroes, maximum continuous non-zero stretch, and variance.

6. A method claimed in claim 1, wherein the method of identifying genes using oligopeptides that are found to occur in the ORFs of other genomes but not limited to genomes such as H. influenzae, M. genitalium, E. coli, B. subtilis, A. fulgidis, M. tuberculosis, T. pallidum, T. maritima, Synecho cystis, H. pylori, and SARS-CoV.

7. A method claimed in claim 1, wherein the peptide library data may be taken from any organism but not specifically limited to those used in the invention.

8. A set of genes of SEQ ID Nos. 1 to 44 of H. influenzae, identified by using method of claim 1.

9. A set of proteins of SEQ ID Nos. 170 to 213 corresponding to genes of SEQ ID Nos 1 to 44 of H. influenzae, identified by using method of claim 1.

10. A set of genes of SEQ ID Nos. 45 to 60 of H. pylori, identified by using method of claim 1.

11. A set of proteins of SEQ ID Nos. 214 to 229 corresponding to genes of SEQ ID Nos 45 to 60 of H. pylori identified by using method of claim 1.

12. A set of genes of SEQ ID Nos. 61 to 165 of M. tuberculosis, identified by using method of claim 1.

13. A set of proteins of SEQ ID Nos. 230 to 334 corresponding to genes of SEQ ID Nos 61 to 165 of M. Tuberculosis, identified by using method of claim 1.

14. A set of genes of SEQ ID Nos. 166 to 169 of SARS-corona virus identified by using method of claim 1

15. A set of proteins of SEQ ID Nos. 335 to 338 corresponding to genes of SEQ ID Nos 166 to 169 of SARS-corona virus, identified by using method of claim 1.

16. Use of proteins of SEQ ID Nos. 170 to 338 corresponding to the genes of SEQ ID Nos. 1 to 169, as the drug target for the managing disease conditions caused by the pathogenic organisms in a subject in need thereof.

17. A use as claimed in claim 16, wherein the pathogenic organisms are selected from a group comprising SARS-corona virus, H. influenzae, M. tuberculosis, and H. pylori.

18. A use as claimed in claim 16, wherein the use is extended to eukaryotes and multicellular organisms.

19. A use as claimed in claim 16, wherein the subject is an animal.

20. A use as claimed in claim 16, wherein the subject is a human.


SEQUENCE LISTING (373 sequences)

SEQ ID NO: 1 - DNA, 633 bp, Haemophilus influenzae
ttgttgttgaaaggagtgattatgcaggtctcaagaagaaaattcttcaagatctgtgcaggaggtatggcgggaacgtcagctgcaatgttgggctttgctccagcaaacgtattagctgcgccacgcgaatataaattattacgcgcgtttgaatcccgtaacacctgtacatattgcgctgtaagttgcggtatgttgttatatagcacaggcaaaccttacaattcattaagcagccatactggcacaaatactcgttcaaaactctttcatattgagggtgatccagatcatccagtcagtcgtggtgcgctttgcccgaaaggtgctggctcactcgattatgtcaatagtgaaagccgttctttatatcctcaatatcgtgcgccaggttctgataaatgggaacgaatttcttggaaagatgccattaaacgtattgctcgtttaatgaaagatgaccgagatgccaactttgttgaaaaagattcaaatggaaaaacggttaatcgttgggcaacgacaggaattatgactgcatcagcaatgagcaatgaagctgcgttattaacacaaaagtggattagaatgctcggtatggtgccagtatgtaaccaagcgaatacttga

SEQ ID NO: 2 - DNA, 1024 bp, Haemophilus influenzae
atgacaaataactgggttgatattaaaaatgccaacttaatcatcgttcaaggcggtaaccctgcagaagcccatcctgttggcttccgttgggcaattgaagcgaagaaaaacggtgcgaaaatcatcgttattgatccgcgttttaaccgtacagcatccgttgctgatcttcatgcgccaattcgttctggttctgatattacgttcttaatgggcgtgatccgttacctattggaaacaaaccaaattcaacacgaatatgttaaacactataccaacgcatcattcttaattgatgaaggtttcaaatttgaagatggtttatttgtagggtataacgaagaaaaacgtaactacgataaatctaaatggaactaccaatttgatgaaaatggtcacgctaaacgtgatatgacattacaacatcctcgttgtgtcattaacatcttaaaagagcacgtttctcgttataccccagaaatggttgaacgtattacaggcgtaaaacaaaaactcttcttacaaatctgtgaagaaattggtaaaacctctgtgccaaataaaacgatgacgcatctatatgcattaggttttacagagcattcaatcggtacacaaaatattcgctcaatggcgataatccagttacttttaggtaatatggggatgccaggtggcggtattaacgcattacgtggacactccaatgtgcaaggtacgacagatatgggcttattgccaatgtctttaccaggttatatgcgtttgccaaacgataaagatacctcttacgatcaatacattaacgcaattacaccaaaagatatcgttccaaaccaagtgaactattatcgtcatacttcaaaattctttgttagcatgatgaaaactttctacggagataatgccactaaggaaaatggctggggattcgatttcttaccaaaagcagatcgcctatatgatccaattactcacgttaaattgatgaatgaaggcaaattacacggttggattttac

SEQ ID NO: 3 - DNA, 198 bp, Haemophilus influenzae
gtgatgagccgacatcgaggtgccaaacaccgccgtcgatatgaactcttgggcggtatcagcctgttatccccggagtaccttttatccgttgagcgatggcccttccattcagaaccaccggatcactatgacctactttcgtacctgctcgacttgtctgtctcgcagttaagcttgcttataccattgcactaa

SEQ ID NO: 4 - DNA, 180 bp, Haemophilus influenzae
gtgtttatgctttatttagaatttttatttttactattaatgctctatatcggtagccgttacggcggtatcggattaggtgttgtttctggtatcggtcttgctatcgaggttttcgtatttcgtatgccagtggggaagcaccgattgatgttatgcttatcattcttgcagtggtga

SEQ ID NO: 5 - DNA, 300 bp, Haemophilus influenzae
atggctgctgcaattcaacaacgtgccgaacttcaacgccgtatttggcaaattgctaatgatgtgcgaggctcggtcgatggctgggatttcaaacaatatgtgcttggcacacttttttaccgttttattagcgaaaattttgccaattacattgaagcgggcgatgaaagcgtaaattatgcccaattacctgatgaaatcattacacagatgccattaaaacgaaaggctactttatttacccaagccaattatttaagaatgttgcggctaatgctggcagcaatcctaatttga

SEQ ID NO: 6 - DNA, 822 bp, Haemophilus influenzae
ttgaatactgatttaaaacagatttttactgatattgaaaactcagcgacgggctttccgtctgaacaagatattaaagggttatttgccgattttgataccaccagcaatcgcttaggcaataccgtaaaagataaaaacgaccgcttaacggctgttttgaaaggcgtggctgaacttgattttggcaaatttgaagataaccacattgatttatttggcgatgcatacgaatatcttatttctaactatgccgccaatgcaggcaaatctggtggcgaattttttaccccacaaagtgtttccaaactcattgctcaaattgcaatgcacgggcaaacctcggtcaataaaatttatgaccctgcagcaggttctggctcacttttgcttcaagccaaaaaacaatttgatgaacatattattgaagaaggctttttcgggcaggaaattaaccataccacatacaaccttgcccgtatgaatatgtttttgcataacatcaactacgacaagtttgatattgctttaggcaacaccttaatggaaccacaatttggcgataataaacctttcgatgccattgtttcgaacccgccttactccgtgaaatgggctggctccgacgatccaacattgattaatgatgaacgatttgccccccgcaggcgtgcttgcaccaaaatccaaagcggactttgcctttattttacatgcgttaagttatctttcagcaaaaggccgcgcggcgattgtttccttccctggtattttttatcgtggcggtgccgagcaaaaaattcgtcaatatttggtggataa

SEQ ID NO: 7 - DNA, 327 bp, Haemophilus influenzae
atgatgaacgatttgccccccgcaggcgtgcttgcaccaaaatccaaagcggactttgcctttattttacatgcgttaagttatctttcagcaaaaggccgcgcggcgattgtttccttccctggtattttttatcgtggcggtgccgagcaaaaaattcgtcaatatttggtggataataactatgtggacgcggtgattgcgcttgcgccaaatctcttttttggcaccagtattgcggtgaatattttggtgctttccaaacacaaacccaatttatcgatgccagcggtttatttaaatctgccactaataaccacattttag

SEQ ID NO: 8 - DNA, 204 bp, Haemophilus influenzae
gtgccgcatttggcaaaatccatatcctttgaagaaatcgcccaaaatgactacaaccttgcagtaagttcgtatgtggaacaaaaagacactcgtgaagtgattaatattgatgaactcaatgctcaaattcgtgaaactgttaccaatattgaccacttgcgtgcggaaattgacaagattgttgcagaaattgaagggtaa

SEQ ID NO: 9 - DNA, 492 bp, Haemophilus influenzae
atgacccaatacaaaactatcgctgaatccaataattttatcgttttagatcaatataataaatttgtggaagaatctaatgctggttatcaaacggaaaggagccttgagcgtgagtttattcgtgatttacaggctcaaggctatgagtatttacaatggcttaataatcacgatgaactgattaaaaacttacgggcgcaattacaacgcttaaataacgtggttttctccgatgcagaatggcaacgttttttagaggaatatttggataaaccgagcgataatctgattgagaaaacccgcaaaattcacgatgattatatttatgattttgtgttcgataacggacgcattcagaacatctatttgcttgataagaaaaatcttgccaataattctctgcaagtcatcaatcaatttaagcaaactggcagctatgataatcgttatgatgtgacaattttggtgaatggtttacccctttattga

SEQ ID NO: 10 - DNA, 1024 bp, Haemophilus influenzae
atggtttacccctttattgaattaaaaaaacgcggcgtggcgattcgtgaagcctttaaccaaattcaccgttacagcaaagaaagtttcaataaagaaaattctctctttaaatatattcagatttttgtcatttctaatggcacggatactcgctattttgctaatacgactaaacgcaataagaatagctacgacttcacaatgaattgggcaacggcaaaaaatactctgattaaagatttaaaggattttaccgcgactttcttgcaaaagaatactttgctcaatgtgttggtaaattactgcgtgtttgatgtgagtgatacgttgttaattatgcgtccgtatcaaattgccgcaacagaacgtattttatggaaaattcaaatttcttacttagcaaaaaattggagtaatcgtgaaagtggtggctatatttggcataccacaggttcaggcaaaaccctcaccagttttaaagcctctcgccttgcgactgaacttgattttattgataaagtcttttttgtggtcgatcgtaaagacttagactaccaaacgatgaaagaatatcagcgtttttcgcctgatagcgtgaatgggtcggaaagtaccgctgggcttaaacgcaatattgaaaaagatgataacaaaattatcgtaaccaccattcaaaaattgaataatttaatgaaaagtgaagaaaacctgtctatttatcaaaaacaggtggtctttattttcgatgaagcacatcgctctcaatttggcgaagcacaaaaaaatctaaaacgtaaattcaaaaaattctatcaatttggttttactggcacgcctattttccctgaaaacgcattaggtgcggaaacgacagcaagtgtgttcggtgcggaattgcattcttatgtgattaccgatgctattcgtgatgacaaagtactgaaattcaaagtcgattacaacgatgtccgcccacaatttaaagccttagaaacagaaaaagatcctgaaaaattgaccg

SEQ ID NO: 11 - DNA, 1024 bp, Haemophilus influenzae
atggatataataaagcctatatgcacaggttttttttataacgataataatgttttaggagatttgatgaaaaatttcaaatattttgctcagagttatgtggattgggttattcgtcttgggcgtcttcgtttttctcttttaggcgtgatgattctcgcggttttagctctttgtactcagattttatttagtctatttattgttcatcagatatcttgggtagatatttttcgttcggtaacttttggcttactcactgcgccttttgttatttattttttcactttattagtagaaaaacttgaacattctcgtcttgatctttctagctcggttaatcgattggaaaatgaggtcgccgagcgaattgctgctcagaaaaaattatcccaagcattggaaaagttagaaaaaaatagccgtgataaaagtaccttacttgccacaataagccatgaatttcgcacgccattgaatgggattgtcgggcttagccagattttacttgatgatgaattggatgatctccagcgtaattatttaaaaactatcaacataagtgcggtcagtttaggctatatttttagcgatattattgatttggaaaaaattgatgccagccgaattgaattaaatcgccagccaacagatttccctgccttattaaacgatatttataattttgctagtttcctcgccaaagaaaaaaatcttattttttctttagagcttgaacctaatttgcctaattggttgaatcttgatcgtgttcgcttgagccaaattttgtggaacttaattagtaatgcggtgaagtttacggatcagggaaatattattcttaaaattatgagaaatcaggattgttaccattttattgtgaaagatacaggaatggggatttcacctgaagaacaaaaacatatttttgaaatgtattatcaagtgaaagaaagccgccagcaaagtgcgggtagcggtattgggttggctatttctaaaaatcttgctcagttaatgg

SEQ ID NO: 12 - DNA, 198 bp, Haemophilus influenzae; identical to SEQ ID NO: 3

SEQ ID NO: 13 - DNA, 1024 bp, Haemophilus influenzae
gtgaatattcatggtttagcaaaacttaatggtaatgtcactttaatagatcacagccaatttacattgagcaacaatgccacccaaacaggcaatatcaaactttcaaatcacgcaaatgcaacggtaaataatgccacgttaaacggcaatgtgcatttaacggattctgctcaattttctttaaaaaacagccatttttggcaccaaattcagggcgacaaagacacaacagtgacgttggaaaatgcgacttggacaatgcctagcgatactacattgcagaatttaacgctaaataatagtactgttacgttaaattcagcttattcagctagctcaaataatgcgccacgtcaccgccgttcattagagacggaaacaacgccaacatcggcagaacatcgtttcaacacattgacagtaaatggtaaattgagcgggcaaggcacattccaatttacttcatctttatttggctataaaagcgataaattaaaattatccaatgacgctgagggcgattacacattatctgttcgcaacacaggcaaagaacctgtgacccttgagcaattaactttgattgaaagcttagataataaaccgttatcagataagctcaaatttactttagaaaatgaccacgttgatgcaggtgcattacgttataaattagtgaagaataagggcgaattccgcttgcataacccaataaaagagcaggaattgctcaatgatttagtaagagcagagcaagcagaacaaacattagaagccaaacaagttgaacagactgctgaaaaacaaaaaagtaaggcaaaagcgcggtcaagaagagcggtgttgtctgataccccgtctgctcaaagcctgttaaacgcattagaagccaaacaagttgaacagactactgaaacacaaacaagtaagccaaaaacaaaaaaagggcggtcaaaaagagcattgagtgcagcgttttctgataccccgtttgatctaagccagttaaaggtattcgaag

SEQ ID NO: 14 - DNA, 1024 bp, Haemophilus influenzae
atgaaaaaaactgtatttcgtcttaattttttaaccgcttgtgtttcattagggatagcatcacaagcctgggcaggtcatacttattttgggattgactaccaatattatcgtgattttgccgagaataaagggaagttcacagttggggctaaaaatattgaggtttataacaaagaagggcaattagttggcacatcaatgacaaaagccccgatgattgatttttccgtggtgtcgcgtaacggcgtggcggcattagtaggcgatcagtatattgtgagcgtggcacataacggcggatataacgatgttgattttggtgcagaaggacgaaaccctgatcagcaccgctttacttatcaaattgtaaaaagaaataattatcaagcttgggagagaaagcatccttatgatggagattatcatatgcctcgtttacataaatttgtaactgaagctgaacctgtgggtatgacaacaaatatggatggaaaagtatatgctgatagagagaactatcctgagcgtgtacgtataggctcaggacgtcagtattggcgtacagataaagatgaagaaacgaatgtacatagttcatattatgtctcaggtgcatatcgttatcttactgcaggaaatacccatactcagagtggaaatggtaatggtacagtcaatcttagtggtaatgtagttagccctaatcattatggtccattaccaacgggtggttctaaaggcgatagcggttcgccaatgtttatttatgatgcgaagaagaaacaatggcttataaatgctgtattacaaactgggcatccttttttcggaagaggtaatgggtttcagttaatacgtgaagaatggttttataatgaagttcttgcggttgatacccctagtgtttttcaacgctatattcccccaataaatggacattattcctttgtatcaaataatgatggtacaggtaaattaactttaactagacctagtaaagatggctctaaagcaaaatcag

SEQ ID NO: 15 - DNA, 396 bp, Haemophilus influenzae
gtgggggaaaacgcgatgaatttaagtcgtcgagactttatgaaagccaatgcggctatggcagccgcaacggcagcggggctaaccatcccagtcaaaaatgtggttgcggctgaatccgaaattaaatgggacaaagcagtatgtcgtttctgtggtaccggttgtgcagtattagttggtactaaagatggacgtgttgtggcatctcaaggcgatcctgatgcagaagtaaaccgtggtttaaactgtattaaaggttatttcttgccaaaaattatgtacggtaaagaccgtttaacgcagccgcttttacgtatgacaaacggaaaatttgataagaacggcgattttgcgccagtttcttgggattttgccgttcaaaacaatggctga

SEQ ID NO: 16 - DNA, 1024 bp, Haemophilus influenzae
ttgataagaacggcgattttgcgccagtttcttgggattttgccgttcaaaacaatggctgaaaaattcaaagaagcgttcaaaaagaacggtcaaaatgcagtaggtatgtttagttctggtcagtctaccatttgggaaggctatgcaaagaacaaactttggaaagcaggttttcgttctaacaacgtagacccgaatgcgcgtcactgtatggcatctgcagcggttgcgtttatgcgcaccttcggtatggatgaacctatgggttgttataacgacattgaacaggcagatgcttttgttctttggggctcaaatatggcggaaatgcacccaattttgtggtcgcgtattactgatcgccgtatttctaatcctgatgttcgtgtcactgtactttctacttacgaacatcgtagttttgaacttgccgatcacggtttgatatttacaccgcaaactgatttggcaattatgaactacatcatcaattatcttattcaaaataatgcgattaattgggattttgttaataaacataccaaatttaaacgcggagaaacgaatattggctatggtttgcgtccagagcatccattagaaaaagacacgaatcgtaaaacagctgggaaaatgcacgattcttcttttgaagaattaaagcaacttgtatcagaatatacagtggaaaaagtatcgaaaatgtctgggttagataaagtccagttagaaactttagcgaaactttatgctgatccaacgaagaaagtggtttcctactggacaatgggctttaaccaacatacacgtggtgtgtgggtaaaccaattaatctacaatattcatttacttactggaaaaatttcaatcccaggttgtgggccattttcattaactggtcagccttctgcttgtggtacggcgcgtgaagtaggttcattccctcatcgtttacctgccgacttagtggtaactaatccgaaacaccgtgaaattgctgaacgtatttggaaattaccaa

SEQ ID NO: 17 - DNA, 198 bp, Haemophilus influenzae; identical to SEQ ID NO: 3

SEQ ID NO: 18 - DNA, 651 bp, Haemophilus influenzae
ttggttatgttcaatgattttttggcaacattcagccagcaattaacacctcaaatgtggggcgttgtcgcaaccgcaacttatgaaactgtttatatcagttttgcatctaccctacttgctgtactagtcggcgtgcctgttggcatatggacttttttaactggaaaaaatgagattttacaaaataaccgcactcattttgtgttaaacacgattattaatattgggcgttccattccatttattattttgctcctaatcttattacctgtaactcgtttcatcgtgggaactgtattaggtacaacagcagcaattattccattgagtatttgtgcaatgccattcgtggctcgcttaactgctaatgcactaatggaaattccaaatggtttaaccgaagcagctcaagcaatgggggctactaaatggcaaattgttcgtaaattctatttgtcagaagctctacctacgctaattaatggcgttactcttacgctagtcactttagttggttattctgcaatggcaggaacacaagggggcggtggtttaggtagcctcgctatcaactacgggcgtatatcgcaatatgccttatgtaacttgggtggcaaccattattattgtgctattcgttatgattag

SEQ ID NO: 19 - DNA, 198 bp, Haemophilus influenzae; identical to SEQ ID NO: 3

SEQ ID NO: 20 - DNA, 138 bp, Haemophilus influenzae
ttgcgtaaagatgcactacccgcatttttcacagacgtaaatcaaatgtatgatgccttattgaataaatcaggggcaacaggtgtatttactgatttcccagatacttgcgtggaattcttaaaaggaataaaataa

SEQ ID NO: 21 - DNA, 198 bp, Haemophilus influenzae; identical to SEQ ID NO: 3

SEQ ID NO: 22 - DNA, 513 bp, Haemophilus influenzae
ttgcctaaacctgaaccaataccacgaccgaggcgtttagcactatgctttgcaccttcagccggagatagagtatttaaacgcatctcttactcctccactttaaccatgtatgaaacttggttaatcataccacgtactgcaggcgtatcaattaactcaacagtgtggtgtatatggcgaagaccaagaccacgcaaggtagctttatgcttcggtaaacgagcaattgagctacgaacttgtgttactttaatagttttagccattattcattaccccaagatttcatcaacagttttaccgcgttttgcagcaaccatttctggtgatttcatatttgctaatgcatcaatagttgcacgaacaacgttaattgggttggtagaaccatacgctttagaaagaacgttacgtacacctgcaacttccaataccgcacgcattgcaccaccagcgatgatacctgtaccttcacttgctggctgcataaatacacgtgaaccagtatga

SEQ ID NO: 23 - DNA, 1008 bp, Haemophilus influenzae
ttgtttatatatgggggaataaatatgcaaattacactttcaaataccttagcgaatgatgcttggggaaaaaatgcgattttgagctttgactctaataaagctatgattcatttaaaaaataatggaaaaactgaccgcactttagttcaacaagctgctcgtaaattgcgtgggcaaggaatcaaagaggtggagttggtcggcgagaaatgggatttggaattttgctgggcgttttatcaaggtttttataccgcaaaacaagattacgcgattgagtttccacatttagatgatgaaccgcaagatgaattgttagcacgtattgaatgtggcgattttgtgcgtggaattattaatgaaccagcacaaagtttaacgcctgtgaaattagtagagcgagcggctgaatttatcttaaaccaagcggacatttataatgaaaaaagtgcggtaagttttaagattatttctggcgaggaacttgagcaacaaggttatcacggaatttggactgtgggtaaaggctctgcgaacttgccagccatgttgcaacttgatttcaatccaacacaggattcgaatgcgcccgtgttagcttgtttagttggtaaggggattacttttgatagtggcggctatagtatcaaaccaagtgatggtatgagtacaatgcgaactgatatgggcggggctgcattattaacgggggctttaggtttcgctatcgctcgtggattaaatcaacgcgttaagctgtatttatgttgcgcagaaaatttggtaagcaataatgcctttaagctaggcgatattattacttataaaaatggcgtgagcgcagaagtactgaatactgatgcggaaggtcgtttggtgttagctgatggattgattgaggctgataaccaaaatccaggttttattattgattgcgcgactttaactggcgcagcaaaaagtggctgtaggaaacgactatcattctgtattatctatggatga

SEQ ID NO: 24 - DNA, 366 bp, Haemophilus influenzae
gtggctgtaggaaacgactatcattctgtattatctatggatgatgaacttgtgaaaaatcttttccaatccgcacaagcagaaaatgaacctttctggcgtttaccatttgaagattttcatcgttcacaaattaattcatcttttgccgatattgctaatattggttcggttccagttggagctggggcaagcactgcaacggcatttttatcgtattttgtaaaaaattataaacaaaattggttgcatattgattgctccgcgacttatcgtaaatctggtagtgatttatggtctgttggggcaacaggaattggtgtgcaaactttagctaatttaatgttatcaagatcattgaagtaa

SEQ ID NO: 25 - DNA, 1024 bp, Haemophilus influenzae
ttgccaattgaattaaaagtagaaggtttagtgggtaaaccaaacgagaaaatttctgcggcagaatttcgtcaaaaatgtcgtgaatacgcggcggaacaggtcgagggtcaaaagaaagactttatccgtttaggtgtgttgggcgattgggataatccatatctcacgatgaatttcgataccgaagcgaatattatccgcactttaggtaaagtgattgaaaatggtcatttgtataaaggctcaaaaccagttcactggtgtttggattgcggttcttctttagcagaagcagaagtggaatatgaagacaaagtttctccgtcaatttacgttcgtttccctgcggaaagtgcggatgaaattgaagctaaattttctgcacaaggtagaggacaaggtaaattatcagccatcatttggactaccacaccttggacgatgccatctaaccgtgcgattgcggtgaatgcagacttagaatacaacttagtccaacttggcgatgagcgtgtaattttagctgctgaattagttgagtcagtggcaaaagcggtgggtattgagcacattgaaattctgggttctgtaaaaggtgatgatcttgaattaagccgtttccatcatccgttctatgattttactgtgccagtgattttaggcgatcacgtaaccactgatggcggtacaggtttagtacataccgcacctgatcacggtttagacgactttatcgtgggtaaacaatatgatttaccaatggcgggtcttgtatcgaatgatggtaaatttatttcaacgaccgaattctttgcaggcaaaggcgtatttgaagcaaatccgcttgtgatagaaaaattacaagaagtaggtaacttattaaaagttgaaaaaatcaaacacagctatccacactgctggcgtcacaaaacgccaattattttccgtgcaacaccgcaatggtttatcggcatggaaacgcaaggtttacgccaacaagcattaggcgaaattaaacaag

SEQ ID NO: 26 - DNA, 102 bp, Haemophilus influenzae
ttggaaaataaaatgacagtcgattacaaaaacactcttaacctaccggaaaccagctttccaatgcgcggtgatttagctaagcgcgaacctgataagtag

SEQ ID NO: 27 - DNA, 108 bp, Haemophilus influenzae
atgaagataactcattgtaaattaaagaaatctatacaaaataagctacttgaattttttgtattagaagttacagcccgagcagcggctgatttactcgatatctaa

SEQ ID NO: 28 - DNA, 504 bp, Haemophilus influenzae
ttgtttctggttggaaaccttttgaggtgggtttggcttgcgctttttatcattgcgcaaatttgggcttatgtacaaacacctgattcttggttagcaatgatttctggtatttctggtattttgtgtgtggtattggtaagtaaaggtaaaattagtaattatttctttggattgatttttgcctatacttatttttatgttgcttggggatcgaatttcttaggcgaaatgaacaccgtactttacgtatatttgccctctcaatttattggttactttatgtggaaagccaatatgcaaaatagcgatggtggagaaagcgtgattgcaaaagcgttaactgttaaaggatggatgacattaattgttgtgactacggttggtactttgctttttgttcaagcattacaagcggctggtggtagctcaacaggtttagatggtctaactacaattattacggttgcggcacagattttaatgattttgccgttatcgtga

SEQ ID NO: 29 - DNA, 747 bp, Haemophilus influenzae
atgtttagtggcgaacatgatgcttgcgattgctatgtggacctacaagcaggttctggcggcaccgaagctcaagattggacagaaatgttgctccgtatgtatctccgttgggctgaaagcaaaggttttaaaacagaactgatggaagtctctgacggcgatgtagctggattgaaatcagcaaccattaaagtgagcggtgaatatgcttttggttggttacgaacagaaacggggattcatcgtttagtgcgtaaaagtccatttgattccaataaccgtcgtcacacatcattcagcgcagcatttgtctaccctgaaattgatgatgatattgatattgaaatcaatcctgctgatttacgtattgatgtttatcgtgcatcaggggcaggtggtcagcacgtaaacaaaactgaaagtgcggtgcgaattacccatatgccaagtggcattgtggtgcaatgtcaaaacgaccgttcacagcacaagaacaaagatcaagcaatgaaacaattaaaagcgaaattgtatgagcttgaattacaaaagaaaaatgcggataaacaagcaatggaagataataaatctgacattggttggggaagccaaattcgctcttatgtattagacgattcacgcattaaagatttacgtactggcgtagaaaaccgtaatacgcaagccgtattagacggggatttagatcgatttattgaagcgagtttaaaagcgggcttgtag

SEQ ID NO: 30 - DNA, 246 bp, Haemophilus influenzae
ttgcttggtaacgaaaaacaagctgaagcacaagctaaatatgcggaagacacgctgaaacaagcacgcgattttgctaaacaacatcataaaacagcctatttagcgcgtaatgcggatggcttacaaactggtcaaaaaggttcgattcatacggaagcaatggaattggttggcttggaaaacgtcgcagagggagaacaaaaaggcttaactcaagtttcaatggaacagcttttattgtga

SEQ ID NO: 31 - DNA, 537 bp, Haemophilus influenzae
ttgccacgtatttttgccgcttgttttgtcggggcggcgcttgcttgtgggggcgcaacttatcaaggtatgtttaaaaatccgcttgtttcgccagatattttgggtgtttcagcgggggcaggttttggggcaagtttggcaattttttataatttgccaatgatttatatccaattttttgcttttagcggtggcattttagctgtgttatgtgtatcgctcattgcctcgcgtagtcgtacacaagatcctattttagtgctggtgctttctgggattgcaattggttctttacttggtgcaggcatttctttgttaaaaattcttgcggatcctttcactcaattaccttcaatcactttttggctacttggtagcctgacggctattaatcaacaagatttaattcaattgatcccgatgttgttgctagggattgttcccatttttttattacttactgatacgctggctcgcacgattgcaccgattgaactgccactcggtattctgacttctgcttgtggttattag

SEQ ID NO: 32 - DNA, 201 bp, Haemophilus influenzae
  misc_feature (29)..(30): n is a, c, g, or t
ttgaagaactcattacgggagttaaaacnngattatactgtggttatagtaactcataatatgcaacaagctacacgttgctccgactatacggcatttatgtatttgggtgaattagttgaatttggtcaaacacaacaaatttttgatagacccaagatacaacgtacagaagattatattcgcggtaaaatggggtag

SEQ ID NO: 33 - DNA, 627 bp, Haemophilus influenzae
  misc_feature (597)..(598): n is a, c, g, or t
atgattagtctacaagaaaccaaaatagctgtgcaaaatctaaatttctactatgaggattttcatgcattaaaaaacattaatttacgtatcgctaagaataaagtgaccgcctttattggtccttcaggttgcggtaaatctactttattgcggagttttaatcggatgtttgaactatatccaaatcaaaaagctactggtgaaattaatttagacggtgaaaatttactcacaacaaagatggatatttctctgattcgtgctaaggttggtatggttttccaaaaaccaacgccatttccaatgtcgatttatgataatattgcattcggtgttcgtttgtttgaaaaattatcaaaagaaaagatgaatgaacgagtagaatgggcattgactaaggccgctctttggaatgaagtgaaagataaattacataaaagcggagatagtttatctggcggacaacagcaacgcttgtgcattgctcgagggattgctattaaacctagtgtgttgttgttagatgaaccttgttcggcattagatcctatttcgactatgaaaattgaagaactcattacgggagttaaaacnngattatactgtggttatagtaactcataa

SEQ ID NO: 34 - DNA, 198 bp, Haemophilus influenzae
atgagccagcttaatattcaatttccgacaaaattcaaaccgctctttgaatctatttggcggtttattattttctacggtgggcgaggttcaggtaaaagttttagtatcgctagagcattagtattgcgagcctatcaatcgcctgttcgagttttgtgttccgtgaaattcagaaatcgatttctgattctgtga

SEQ ID NO: 35 - DNA, 861 bp, Haemophilus influenzae
  misc_feature (578)..(578): n is a, c, g, or t
gtggttcccgagttcattattgtttctttaatcttggtggcacagtccatgaaattggcgttaaacaaatggcttatcatatttggcaacgctatagctcttcacataaagtacgctttattgcgattaaactttgagggagttgttggtgagattttagagaaagtcgataacggccaaatgggcgttgtattaaaacggatgatggtgcgagccgcaagtaaagtcgctcaacgtttcaatattgaagcaattgtgacaggggaggcattagggcaagtttctagccaaactttaaccaatttacgcttgattgatgaagccgctgatgccttagtattgcgtccgttaattacccatgataaagaacaaattatcgcgatggcgaaagaaattggcactgatgatattgcaaaatctatgccagaattttgtggcgtgatttcaaaaaatcctacgattaaagcggttcgtgaaaagattcttaaagaagaagggcattttaattttgagattcttgaaagtgcggtacaaaatgcaaaatatttagatattcgccagattgcagaagaaacagnaaaagcagtcgtggaagtcgaggcaatttctgtgttaggtgaaaatgaagtgattttggatattcgtagcccagaagaaacggatgaaaagccatttgaatcaggtacacatgacgtcattcaaatgccgttctacaaactttcttctcaatttggtagccttgatcaaagtaaaagttacgtgttgtattgtgaacgtggtgtgatgagtaaattacaagccttatatttgaaagaaaatggtttttcaaatgtgcgtgtatttgcaaaaaacattcattaa

SEQ ID NO: 36 - DNA, 327 bp, Haemophilus influenzae
ttggccatcgctattggtggaggtaatagaggtaatgcaagcggagtattgcgccaaaattttgcagaagataaagcaaaaaagaccgcttcgaagctcgtgggcgtaatggctcactattttggcggtaagtcgttttatctgcccgcaggtgataaaatcaaagaagccttacgagatgcacaaatttatcaagaattcaacggtaagaatgtacctgacctaataaaaaaataccgattgtcagaaagcacaatttatgcgatcttacgcaatcaacgaacgcttcaaagaaagcgacatcagatggattttaattttagttag

SEQ ID NO: 37 - DNA, 822 bp, Haemophilus influenzae
ttgtttaggtggcactaccttggaggttttacagtaatgccagatacaaataacacagaaaccaataataagatcgaactctatctaaatggcaaaattttatccggttggaaaagccttaacctgcaacgctcgctggaatcaatgagtggtcgttttgatttaggcattgctgtgcgacctgaagatgatatatcagtgcttgccgcaggttcgccactggtgctgaaaatgggcgggcaaaccgtgattaccggttacttggatgaaatcaaacaacgcgtaagcggtaacgacaaaactatctctgtgagtggacgagataaaacttgcgacttggtggattgtgccattatccacaacagctaccaattcaaaaaccaaactgccaaacaaattgccgaagccatctgtaaaccttttggcattagcgtagtatggcaagtgcaagcccctgaagccaatgaacgaatccctgtctggcaagtagaaccaggcgaaaccgcctttgataatttaagcaaaatcgcccgacacaaaggcgtgttagtcaccagcgacgtggacggcaatttgcttttcaccgagccgagcaacaagcaagtcggtaatcttacccttggcgaaaacttgctcgaactggaacaaaccgacagctggttgcaacgcttttcgctctatcgcgtgattggtgacgcagaacaaggcggcgccaaaggtgataccaaaaccaaaaacaaagcggcaaaaggcaaggaaaaagatgatggcgtggtagaagatcccgatatttacccaggaccagcagaaggaggcaagtaa

SEQ ID NO: 38 - DNA, 516 bp, Haemophilus influenzae
atgaaggtttcttaccggctaaataattgtctaagtttaaagttagcgctgatcccattattaatactattatttgttgttatgggatcggtgctttctttaatcgcaaaattagatttttatttttttcaacaaatattatttaattccgaattgcattttgcattgctaatgtcattgggaacgtctcttttttctttgatattagcattatgtattgctattccatctgcatggcgaatgagtcaagtgcggttgccttttcaatcattttttgacactttgtttgatttaccaatggttttgccaccattagtcacaggactaagtttgcttctactttttagttcacaagggatattggctgaactacttccttttataagtaaatggattttttcccctgtagggatcattattgctcagacttatattgcgagttcgattttattgcgttgtagcgagccattaaaactgcgaaaaaaaaccattaaaactacgaaaataaaaccttga

SEQ ID NO: 39 - DNA, 1024 bp, Haemophilus influenzae
ttgacaaaacgtaaaaatgtttcctttacttatgaaaattatactgttacgccattttgggatacgctcaagttaagctattcacaacaaagaattacaacaagagcaagaacagaagattactgtgatggtaatgaaaaatgtgactcttataagaatcctttagggcttcaattaaaagagggaaaagtcgttgatcggaatggtgatcctgttgagttgaagcttgttgaggatgaacaaggtcagaaacgacatcaagttgttgataaatataataatccttttagtgtagcctctggaactaataatgatgctttcgtaggtaaacaattatctccttctgagttttggttagattgctctatttttaattgtgataagcctgtcagggtttataaatatcagtatagcaaccaagaaccagagtcgaaggaagttgagttaaatagaaccatggaaattaatggaaagaaatttgctacttatgagtctaataattatagagatagataccatatgattttaccaaattctaaaggttacttgcctttggattataaagagcgtgatttaaatacaaagacgaaacaaattaatttagatttaacaaaagcctttactctctttgagattgaaaatgaactttcctatggtggtgtttacgcgaaaacgaccaaggaaatggtgaataaagcaggatattatgggcgtaatcctacttggtgggcggagagaacgttagggaaatcattgcttaatggattgagaacgtgtaaggaagattcttcatataatgggctactatgtcctcgtcatgaacctaaaacgtctttcttaattcctgtagaaacaacaactaagtctttatattttgcagacaatatcaagttgcacaatatgttgagcgtagatttaggttatcgttatgatgatattaaatatcagccagagtatattcctggtgtaacacctaagattgcagatgatatggtcagagaattatttgttccactccctccag

SEQ ID NO: 40 - DNA, 747 bp, Haemophilus influenzae
ttgcgtgaacgtagttcgctttctgctctaatggccaaaacgattgaatgggattttataacagaaaaccccctaaaatatcttgagaaaccaaaagcgccagcaccaagaactcgtcgatataatgaacatgaaattgagcgtctgatttttgtgtcaggttatgatgtcgaacatattgaaccgccaaaaaccttacaaaattgcacgggggcggcatttctttttgctatagagacagcaatgagagcaggggaaatagcaagtttaacttggaataatattaattttgaaaagcgcaccacctttttgccaattactaaaaatggacattcacgcacggtgcctctttcggtaaaagcaatagagattttacaacatcttacttcggtaaaaacagaaagtgatccgcgagtattccaaatggaagcacgccaactggatcacaacttccgcaagctcaaaaagatggaagggcttgaaaatgccaatttacattttcacgacacccgccgtgaacgattggcagaaaaagtggatgtaatggtattagccaaaatatcgggccatagagatctcagtattctgcaaaatacttattacgcacctgatatggcagaaggctataaaacaaaggcgggttatgatctgaccccaaccaaaggcttgagccaacggaattttttcttctttaatgaaaacttcatcgttttcacaacaaatccaccgatagtcattaagctgtaa

SEQ ID NO: 41 - DNA, 318 bp, Haemophilus influenzae
atggcgacaattatcaagaatggcaagcgttggcacgcacaagtgcgcaagtttggcgtgagcaaatcagccatttttttgactcaagcagacgcaaaaaaatgggcagaaatgctcgaaaaacagcttgaatcaggaaagtataatgaaatccctgatattacattggatgaactcattgataagtatctaaaagaagtcactgtaaccaagcgcgggaaacgtgaagagcgcataagactactgcgtctttctcgaactccgcttgccgcaatatctttacaagaaataggaaaagcacactttcgtgagtggtaa

SEQ ID NO: 42 - DNA, 1024 bp, Haemophilus influenzae
atggaagccgttcaattagacaaaaatcaagagcctaattataaaggttatagcggtagcttgattcatcctgcatttcaacagcaaacaacaaaacgtgaaaaaccgagtacaccattacctagtttggatttgcttttaaaatatccgccaaatgaacaacgcattacaccagatgaaataatggaaacctcacagcgtattgaacaacaattacgcaattttaatgtaaaagccagcgtaaaagatgtgcttgttggccctgttgttacgcgttatgaattagaattacagccgggtgtgaaagcatcaaaagtcacgagcatcgataccgatttagcaagagcattgatgtttcgttctattcgtgtggcagaggtgattccaggtaaaccttatattggtattgaaaccccaaatcttcatcgtcaaatggtgccattacgtgatgtattagatagcaatgaattccgtgatagcaaggcaactttacctattgctttaggtaaagatattagtggcaaaccagtcattgttgatttagcgaaaatgccacatttattggtagcaggttctacgggatcaggtaagtctgttggtgtgaatacgatgattctaagtttactttatcgtgttcaaccagaagatgtgaaatttattatgattgatcctaaagtcgtcgaactttctgtttataatgatattccacatttactgacaccagttgtaacggatatgaaaaaagccgctaatgcgttgcgttggtgcgtagatgaaatggaacgtcgttatcagttgctttcagctttacgcgtacgaaacattgaaggctttaatgaaaaaattgatgaatacgaagcaatgggaatgcctgtgccaaatccaatttggcgactgggcgatacgatggatgcaatgccaccagcgttgaaaaaattgagttatattgtggttattgtcgatgagtttgctgatttaatgatggtagcgggtaagcaaatcgaagaactgattgcacggttgg

SEQ ID NO: 43 - DNA, 897 bp, Haemophilus influenzae
atgaataaaatttttaaagttatttggaatgttgtgactcaaacttgggttgtggtgtctgaactcactcgcgcccacaccaaacgcacctccgcaaccgtggcaaccgccgtattggcgaccgtattgtctgcaacggttcaggcgattaacgacgcaggaactttcgtgaaagtgcaaagtacggaagatgatattgaagatagtgctgcaaccaaagatgacaataaaaaccaagctctcaaagcaggcgacaccttaaccttaaaagcgggtaaaaacttaaaagctaagttagaccaaggtggtaaatcagtaacctttgctttagcgaaagaccttgatgtgaaaaccgcgaaagtgagtgatactttaacgattggcgggaatacgcctgctgcgggtggtgctacgccaaaagtaagtattactagcacggctgatggcttgaagttagcaaaaggcactaatggagatactgcagttcatttgaatggcttggcttcaactttgcctgatgtgactacaaatacaggtgcctcaacttcagtaaccttttcgcctagtgacattgaaaaaacaagagctgcaactattaaagatgttttaaatgcaggttggaatattaaaggagctaaagttgcggggggtaataccgagaatgttgatttagtggcgggttatgacaatgttgagtttattacaggagataaaaacacacttgatgttgtattaacagctaaagaaaacggtaaaacaaccgaagtgaagttcacaccgaaaacttctgttattaaagataataatggtaagttgcttacaggtaagcagttgaaggatgcgaatactggtacagcgaccaatgcaactgaagatacagacgaggcaatggcttag

SEQ ID NO: 44 - DNA, 198 bp, Haemophilus influenzae; identical to SEQ ID NO: 3

SEQ ID NO: 45 - DNA, 339 bp, Helicobacter pylori
atgtttgcagtgcatgctgcgatgattacgacattaaagaaagaagttttctttctttacctttatatcaaatcactcaaaatcccgattcctactacactgaaatacatgatttctttaggcaaaatcagagaattagatgttttagcaaatcttgctaaactttgccctacttgtcatagggctttaaaaaaaggatctagcgaagaggagtttcaaaaacgcttgattagaaacattctcaatcgcaataaagacaatttagagtttgcgcaattgcgttttgaaaccgatgatttttcaacgcttattgatcgtatttgtgaaagcttgaaatga

SEQ ID NO: 46 - DNA, 798 bp, Helicobacter pylori
atgattaaacaaaccctcatcattcttgcccctttttttatcgcaacgctgttgtattttttaggcgcaccggatgggttaagacctaacgcttggctttatttttgtattttcatgggcatgattatagggctaattttagagccggtgccatcaggtttaatagcgctaagcgcgttagtgctgtgtatagcgttaaaaattggagcgagcgataaagtagcgagcgctaataaggctatttcgtggggtttgagcgggtatgcgaataaaacggtgtggcttgtgtttgtcgctttcattttgggtttagggtatgaaaaaagcttgttagggaaacggatcgctcttttactgattaggtttttagggcaaacccctttaggtttaggctatgcgattggtttgagcgaattgtgtctagccccttttatccctagcaactccgctagaagtggaggcatactctatcccatcgtttcatctatcccgcctttaatgggatctactccaaataataaccctgacaaaatcggcgcgtatttgatgtgggtcgctttggcttcaacttgcatcacttcgtccatgtttttaaccgcgctcgctcctaaccccctagcaatggaaatcgctgccaaaatgggcgtgaatgaaatctcatggttttcgtggtttttagcgttcttgccttgtggggtggttttgatcttgcttgtgcctttattggcgtataaaacctgcaaacccaccttaaaaggctcaaaagaagtgagtttgtgggccaaaaaaaggaattag

SEQ ID NO: 47 - DNA, 219 bp, Helicobacter pylori
atgagccgacatcgaggtgccaaacctccccgtcgatgtgagctcttgggggagatcagcctgttatccccggggtaccttttatcctttgagcgatggcccttccacacagaaccaccggatcactatgaccgactttcgtctctgcttgacttgtatgtcttacagtcaggctggcttgtgccattacactcaacttgcgatttccaaccgcaatga

SEQ ID NO: 48 - DNA, 885 bp, Helicobacter pylori
gtgcaacttcattgccacaacttgccatgcgtttcaattgatattctactaggcggaccaccatgccagagctattctacccttggcaaaagaaaaatggatgaaaaagcgaatctgtttaaagaatatttgcggcttttagatttagtaaaaccaaaaatatttgtttttgaaaatgtggtgggtttaatgtctatgcaaaaagggcaattattcaaacaaatttgtaacgcttttaaagagagagattatattttagagcatgccattttgaacgccctagattatggtgtgcctcaaatgagagaacgagtgattttagtgggcgtgcttaaaagctttaaacaaaaattttacttccctaaacccataaaaacgcatttttctctgaaagacgctttaggggatttaccacccattcaaagcggtgaaaatggtgatgctttaggttatcttaaaaatgcggataatgtttttttggaatttgtgcgaaattctaaagaattaagcgaacatagcagtcctaaaaacaatgaaaaactgataaaaatcatgcaaacgctaaaagacggacagagtaaagatgatttgccagaaagtctgcgtcccaaaagtggttatattaatacctatgccaaaatgtggtgggaaaaaccagcccccaccattacaagaaatttttctaccccaagcagttctaggtgtatccatccaagagactctagagcgttaagcattagagagggggcaagattgcaaagctttcctgataattataaattctgtgggagtggtagcgctaaaagattgcaaattggcaatgccgtgccgcctttattgagtgtagcgctcgcgcaggcggtctttgactttttaaaggggtaa

SEQ ID NO: 49 - DNA, 270 bp, Helicobacter pylori
ttgatggaatttgatgttaccatcatagatgagacaggcagggccacagcaccagaaatcttgattcctgcacttcgcactaaaaaactgatcttaataggcgatcacaaccagctcccacctagcattgataggtacctcctagaacaattagagagcgatgatattcaaaacttggatgccattgatcgccaattattggaagagagtttttttgaaaatctctataagtatattccagagagtaataaggccatgcttaatgagtaa

SEQ ID NO: 50 - DNA, 555 bp, Helicobacter pylori
atgcctgcttctattggatcgctagttagtcagcttttttataaagagaaacttaagaatggagtgatcaaaaatacctcgcaattttacgatcctaagaatattatccgttggattaatgttgaaggggagcatcaactagaaaaaacaagtagctataacaaaaatcaagttcaaaaaatcatagagcttttagagcaaatcaatcgcgttcttaatcaaagaaaaatcagaaaaaccataggaattatcacaccttataatgcccaaaaaagatgcttgcgatcagaagtggaaaaatacggcttcaagaattttgatgagctcaaaatagacactgtggatgcctttcaaggcgagaaggcagatattattatttattccaccgtgaaaacttatggtaatctttctttcttgatagattctaaacgcttgaatgtagctatttctagggcaaaagaaaatctcatttttgtgggcaaaaagtctttctttgagaatttgcgaagcgatgagaagaatatctttagcgctattttgcaagtctgtagatag

SEQ ID NO: 51 - DNA, 651 bp, Helicobacter pylori
ttgattattgaaacgcaacaagaccccaaagaactacctgagtcttgcaaaataacgccccaaaaaatctcttttaaccaagtggtttttaaaaaaattaaaagaaaactcaaccgcttcattggaagcattttagctcggacagaagtgtataagaatctcgtggcaaaatacgatgaactcacaggaaaatacgaatcattattggcaaaagaggcaaacatcaaagagaccttttgggaaaggcgtgctgatagcgaaaaagaagccttttttttagagcatttttacctcactagcgtgtatgtggcttctacagcaggatactatatcacgcctaagggcgctaaaacctttatagaagccacggagcgttttaaaatcatagagccggtggatatgttcataaacaaccccacttaccatgatgtggctaattttacctatttgccttgccctgtttctttaaacaagcatgctttcaatagcaccattcaaaatgcaaaaaagcctgacatttcattaaaaccccctagaaaatcctattttgataatcttttttatgatcaattaaacactagaaagtgcttaaaagcctttcacaaatacagcagacgatacgctcctttaaaaacccctaaagaggtttaa

SEQ ID NO: 52 - DNA, 882 bp, Helicobacter pylori
ttgatggaaattttagtgttgaatctgggcagttcgtctattaagtttaagttgtttgacatgaaagaaaataagcccttagcgagcggtttggctgaaaaaatcggcgaagaaatagggcagttgaaaattaaatcgcatttgcaccataacgatcaagaattaaaagaaaagtttgtgattaaagatcatgcgagcggacttttaatgattcgtgagaatttaacgaaaatggggattatcaaagattttaaccaaattgacgctatagggcatcgtgtggttcaagggggggataaattccatgccccagttctagtcaatgaaaaagtcatgcaagaaattggcaatctttctattttagcccccttacacaacccggcgaatttagccggtattgagtttgttcaaaaagcgcacccccatatccctcaaatcgctgtttttgacaccgcattccatgccactatgcccagttacgcttacatgtatgcgttaccttatgaattgtatgaaaagtatcaaatccggcactatggtttccataggacttcacaccattatgtggccaaagaagcggcgaagtttttgaataccgcttatgaggaatttaacgcgatcagtttgcatttagggaacggctcaagtgcagccgccattcaaaagggtaaaagcgtggatacttctatggggctaacccctttagaaggcttgattatgggcacaaggtgtggggatattgaccccactgtggtggaatatactgcgcaatgcgcgaacaagagcttagaagaagtgatgaaaatgttaaaccatgaaagcggattgaaaggcatttgtggggataatgagaaacatagaagccagaaaagaaaaaggtga

SEQ ID NO: 53 - DNA, 222 bp, Helicobacter pylori
atgcctaacagccaagtggctgggcaagctagcgtttttattttcccggatttaaacgctgggaacatcgcttataaagcggtgcaacggagcgctaaagccgtggcgatagggcccattttacaaggtttgaataagcccattaacgatttgagtaggggcgctttagtggaagatattattaacaccgttttgattagcgcccttcaagcgcaagattaa

SEQ ID NO: 54 - DNA, 372 bp, Helicobacter pylori
gtgagcctggtttcaagcgtgtttttaatgtgtttagacactcaagtgctagtctttggggattgcgcgattatccctaaccctagccctaaagaattagccgagatcgctaccacttccgcacaaaccgccaagcaattcaatattgcgcctaaagtggccttgctttcttatgcgacaggcgattccgctcaaggcgaaatgatagacaaaatcaacgaagctttaacaatcgctcaaaagttggatccccaattagaaattgatggccccttacaatttgacgcttccattgataaaagcgtagccaagaaaaaatgcctaacagccaagtggctgggcaagctagcgtttttattttcccggatttaa

SEQ ID NO: 55 - DNA, 297 bp, Helicobacter pylori
ttgaaagctgcacatcgtttgaatttaatgggcgcggtaggattgatcttattaggcgataaagaagccattaattcgaaaaatttgaacttgaatttagaaaatgtggaaatcattgatcccaacacttctcattatagagaagaattcgctaaaagcttgtatgaattacgaaaatcaaagggcttgagtgagcaagaagctaagcaattagtgctggataagacttattttgcgaccatgctcgtgcattcaggctatgtgcatgcgatggtttctggggtgaatcacagctga

SEQ ID NO: 56 - DNA, 858 bp, Helicobacter pylori
gtgaaacaaattagtatctcttgcagccatagaaaatattttgttagctttagcgtggaatacgaacaagacattactcccataaaaaacactaaaaatggtgtggggctagatttgaatatccttgatatagcttgttcttgtgagataaacaaccatgacaaactaacggactttaagcaataccaaacagacatgaaagaattactagggatagaaatagatgaagagctggatactaaacgacttatccctacttattccaaattgtattctttaaaaaaatactctaaaaaatttaaaagattacaaagaaaacaaagccgtagggtgttaaagtctaaacaaaacaaaaccaaattaggaggtaatttttacaaaacccaaaagaaattaaaccaagcctttgacaagtctagtcatcaaaaaacagacagataccataaaatcacaagcgaactttcaaagcaatttgaattgatagtagttgaagatttgcaagtaaaaaacatgactaaaagagctaaactcaaaaatgttaaacaaaagagtgggcttaatcaatctattttaaacgcttcattctatcaaatcatctcttttttagactacaaacaacagcataatggcaaattgttagtgaaagttcccccacaatatacgagtaaaacttgccattgttgtgggaatatcaaccacaagcttaaattaaatcataggcaatattggtgtttagaatgcgggtatagagaacacagggacatcaacgctgcgaacaacattttaagcaaagggttaagtctttttggggtaggaaatatccatgcagactttaaagaacaaagcctttcgtgttag

SEQ ID NO: 57 - DNA, 474 bp, Helicobacter pylori
atgaaagtcaataagggttttaaattccgcttgtatcccactaaagaacaacaagataagttgcaacactgcttttttgtctataatcaagcttataatattggcttgaatgaactgcaagagcaatatgaaaccaacaaagattcaccacctaaagaaagaaaatacaaaaaatcaagcgaattagacaatgcgatcaaacaatgcttgagagctagggacttgccctttagcgctgtgatagcccaacaagcacgcatgaatgttgaaagggctttaaaagatgcttttaaagttaaaaacagaggctttcctaaattcaaaaactctaaatccgctaaacaatctttttcgtggaacaatcaaggcttctctatcaaagagagcgatgatgagtgcttcaagacattcactctgatgaaaatgcctttactcatgcgcatgcatagagacttccccctaattttaaagtga

SEQ ID NO: 58 - DNA, 324 bp, Helicobacter pylori
ttgatattcatcacccatttttccacagagcctttacctttacccatcctggtttctaagggtttagcggtcaaaggcttatcagggaatactctaatccacaccttacccgctcttttaatgtgccttgtcatggccacccttgcggattcaatttggcgtgaatcaatcctcccatgctctatggctttaatcgcaatatccccaaacgcaatggagttaccccgatgggctttcccacgattgcgccctttcatttgctttctgtattttgttctttttggcattaacatgattattgcctccctcttctgcttcttctag

SEQ ID NO: 59 - DNA, 219 bp, Helicobacter pylori; identical to SEQ ID NO: 47

SEQ ID NO: 60 - DNA, 588 bp, Helicobacter pylori
ttgaacgccgcatttaaagaaaggcgcttcattctcgtccagttagatgaaaaaattgatcccaaggaagacaaaagcgcttatgatttttgtttgaacaccttaaaatcaccctccccaagcatttttgacatcaccgaagaaaggattaaaagagcgggggctaaaatcaaagaagcttgcgcgcatttagatgtggggtttagagcgtttgaaatcattgatgatgaaacgcatgctaatgataaaaatctcagtcaagcccatcaaaaggatttgttcgcttattctaaccttgatagaatggaaacccaaacgattttaattaagcttttaggctgcgagggtttggagctcactacccctataacttgcttgattgaaaacgccttgtatctggctttaaatacggctttcattgtgggggatatagaaatgagcgaagttttagaaaacttgaaagataaaggggtggaaaaaatcagcatgtatatgcccgctatcagtaacgataatttgtgtttggaattgggcagtaatttgttggatttgaaattagagagtggcgatttaaagattagggggtag

SEQ ID NO: 61 - DNA, 705 bp, Mycobacterium tuberculosis
atgtatatacgtttttatcgcgattctcttgcagagcccgccacagacatatacgcttttgcctatgtttcgttcaacaaggaggccggcacatggcacacccctgcgcaaccgacccggaactatggttcgggtaccccgatgacgacggcagcgacggcgccgctaaggcacgcgcctatgagcggtcggccacccaagcgcggatccaatgcctgcgccggtgcccgctcctacagcagcgccggtgtgctcaacacgcggtcgagcatcgggtggagtacggcgtatgggccggcatcaagcttcccggcggccagtaccgaaagcgcgaacagctcgcggcagcccacgacgtgctgcgtcggattgccggcggcgagatcaattccaggcagctcccggacaatgcggctctgctggcccgcaacgaaggactcgaggtcaccccggtgcccggggtcgtggtgcacctgccgatcgcacaggttggcccacaaccggccgcttgatgcccggtcggcaagcccggcagttgccaaacccagcgtgatcaggctcggctcgcgagttcggcgaagaagtggctcgcctgatcacctaccatcggccaggatctgcgtgtcatcacgacgctcgccaaggaggttgttgtggtgctatcgacggcctttagccagatgttcggaatcgactatccgatag

SEQ ID NO: 62 - DNA, 510 bp, Mycobacterium tuberculosis
ttgatgttctgtgcgtcgcggaaagagatggcgatgtcgaattcgtcttctagctcggtgatcaactggaacagcttgagcgagtcaaaacccaggtcgtcgacgagtacctggttcgcggtgatgccgcggtcggttcgcaagatccgttggatggtggcgttgatggcctctttcatagcgcggctccttgcggggtcaggtcctcggcaaggccggcaaacacgtgcaaggcccggtcgaggtcagattgtcggtggtcggctaggtagctggtgcggaatcccgaacgctcctccggcacggctgggggggccaccgggttcacatacaccccggagcgcatcagccgcagatagcccgcatgcgccacggtcgggttgcccaggatcaccggcacgatcgcggttccgtgatactcggcctgatagccctgccgtgccaggccggtggccatgtactcggccgcggccagcacccgagcccgccggtcgggttcacgccgactga

SEQ ID NO: 63 - DNA, 564 bp, Mycobacterium tuberculosis
gtgccgccaccgatcccgcggtgcgcggcggccagtacttcggacccgatggcttcggtgaaatacggggctacccgaaggtggtggcctccagcgcccagtctcacgacgagcagctgcagcgccgcctgtgggctgtgtccgaagagctcaccggggtcgtctatcccgtcggatgagccggactcaacggcaacggttggtcaacactcgacgatgttgactgcgacgttgatggcgagcccgccggccgaggtttccttgtacttggtgtgcatgtccgcgccggtggcgcgcatggtgtcgatgacctggtcgagggtgacgcgatggatgccgtcgccgcgcaatgccatccgtgcggcgttgatggccttgccggcggaaatcgcgttgcgttcgatgcaggggatctgcaccagcccggcgatggggtcacaggtcaggccgaggctgtgttccatggcgatctcggcggcgttttccacttgtcgcggtgtgccgccgaggatttcagccaatccggcggcggccatggcggccgcggagccgacctcgccctga

SEQ ID NO: 64 - DNA, 792 bp, Mycobacterium tuberculosis
atgatcccgatggacgtgatattcggctgcccgttgtacgccaatttctgtaagccctcggtcgtgaggaagacattggggatcttggccagcgcggtggaattcggcacaatgccaacgacccgcaatctgcgcgcgccgacctcgacagtgtcaccgaggtgtcggcccatcgtgctcgatgccgcgacttcgtccggtttcgacggtgaccgaccctctgagacccgtggcatgccaggtccgtgctcgggcgcgccgaagaccgtgacgtttcgcgtcgacgtgccttctttcatgatcgtccccacgctgcccaacggggccgcggccatgacaccgggttcagcggccactcgggccaggtcaacatcgggaaacggtattgaacccagaaaaggtccagcagcgccggatctgacgacgaatacatcgacacccatggaatcgacggtgtgccgggcctccacccggaagccgttcgcgagtccggtcaaaacaagcgtcatcccgaagatcagcccggtgctgatgatcgtgatgaccaggcggcgctttctccattgcatgtcacgcagggccgcgaagagcattcccagaggctaccaacgtggcgcacttgtggggcctggtcttgacgttttgtggtcagggcgcggcccgctagtggtcgaagaggcgttcggggtggtggtagtcgttggtgtgggcaccgcggtcgaggtggggtggcgggatccattccgtttggccgtcggaccgtttccttgtctgccagcctttcccgactag

SEQ ID NO: 65 - DNA, 846 bp, Mycobacterium tuberculosis
atgtcgcgtgctatccggacaaagccgaaatcagcatcttcccggggtagcgcaggctaccgggtatacctcggccaacgactgggtgtcgctgtattcgcgcagcgagatgatcatcccgtcacgggtctcgaagatgcagacgaacgggctgtcatatcgggtccggtcggcgctcacaccgtcgcaatgcccctcgaccactaccgtttcaccctcgttgacgcagcggatgagttcgatgttgacctcgaagacctgcttgcgccgctcgactgctcgccgaaacgtcttcttgtccaattccgtacgggtgacgatgctccagtaggtgaagtcgttgctgagcagcgcgaagccttcgtcgagatctccgccctcgcagaggctttgcaggaacatccaggccagttcggcttgcgggtcgtcgaacggcgtcatcacatcgccatcttgtctcgggagacagcgtgcggtcaattgacgtggtcgtcgaagcggtggtcaccttcgcgggggcggccggcttcgcgcacaccttggcgccgttgcgtcgcggtcagcaggatccatgctttcgggtccccggtgacggcactatctggcggaccagcttgctgcccaccgggccggtcaccgcgcggatcagccgtgctgggcgcgacgccgcccgttgcgtggcgtggggcagcggtgccgaggagtttgtcgacatggcgcccgccatgctgggcgccgccgacgacgccagcgatttcgtgccgctgcatccggccgtggccgccgcgcaccgccggctgccgaacttgcgcctgggccgcaccggccaggtgctggaagccttga

SEQ ID NO: 66 - DNA, 492 bp, Mycobacterium tuberculosis
gtgcgaccgggccaccgccaggtcgatggatgccgccgtggccaaccgttgtgcggtgctcatgaacgcgtcggcctcgtgcgggttgtcggtgccttcggcctggcgcagcagggctgcgatgcgggccagcatcttgtcgttggtcatggcgccaaaactagtggagggctgcgacaggtcggctcggcctacaaccgctcggtgagccaggcgaccacatcgtcgagcacctggttgcgctccggctcgttgaacacctcgtggtacagcccgggatactccttcagctgcacgtcggccgatcccacacattcgaccaggcgacggctgccctcgatggggatcagccggtcatcggtgccgtgcagcactagcagcggcgcggtcaatgccggtgctcgccgcggcatggtctcgcccacctgcagcagcgcgcggccaatcccggccggaacccgtccgtggtgcacgagtgggtcggtgttgtaa

SEQ ID NO: 67 - DNA, 516 bp, Mycobacterium tuberculosis
gtgtgtaaagcatgtctcggtcaccatacccatcaccaccgaacatctcggcccctacgaaatcgatgccagcacgatcaaccccgaccagcccatcgacacggctttcacccaaaccctcgatttcgccggcagcggcaccgtgggcgcgttccccttcggcttcggctggcagcagagcccgggattcttcaactcgaccacaaccccgtcgtcgggcttcttcaactccggcgccggtggcgcatcgggcttcctcaacgacgccgcagccgccgtgtcgggcctgggaaacgtcttcaccgagacttcgggcttcttcaatgctggcggcgtaggaattcgggcttccaaaacttcggcaacctgctgtcgggctgggcgaacctaggcaataccgtctccggtttctacaacacgagcatgctggacctcgcgacccaagcccttatctccggcttcggcaaccacggagcccgactctccggcatcctcaacaacggtagcggaccctaa

SEQ ID NO: 68 - DNA, 1024 bp, Mycobacterium tuberculosis
gtgcttagcctatccgctggcggcccggaaccgagaatgcgaccaggtcacaacccagtcaccttccacgccgagcagacgaggaatcgcactgcgcggacctcacgcgtgcgattccgcgtctgctcgtcagacaaatcagcccaggatcagcgagtcggcgtcggggctgacgttgaccggcacggtatcgccgtcgtgcacctggccggccaacagcatcttggccagctggtcaccgatggcctgctgcaccagccggcgcaacggccgcgccccgtacaccgggtcgaatccgcgctgcgccaaccagcgcttggccggcagcgagacctgcagctgcagccgccgctgcgccagccgcttgcccagctgcgccagctggatgtcgacgatgcgcaccagctcttcggggttgagaccctcaaagatgagcacgtcgtcgagccggttgatgaactccggcttgaacgtagcgcgcaccgcggccagcacctgctcggcgctgccacccgaccccaggttggacgtcaggatcaagatggtgttgcggaagtcgaccgtgcggccgtgcccgtcggtgagccggccctcgtcgaggacctgcagcagcacgtcgaacacgtccgggtgcgccttctcgatctcgtcgaacagcaccaccgtgtagggacgccggcgcaccgcctcggtcagctgaccgcccgcctcgtatcccacatagccgggcggggcgccgatcaaccgagccacggtgtgcttctcgccgtactcgctcatgtcgatgcggaccatcgcccgctcgtcgtcgaacaggaagtcggccagcgccttggccagctcggtcttgccgacaccggtcgggccgaggaacatgaacgccccggtgggccggttggggtcggacaccccggcccggctgcgccgcaccgcatcagagactgcggtaaccgcggccttctgcccgatgacccgcttgcccagctcgtcttccatgcgcagcagcttggcggtctcgccttcc

SEQ ID NO: 69 - DNA, 1024 bp, Mycobacterium tuberculosis
ttgcttgccgatttcgatgtaggacaacaccttttccagctggtcgttggaggcctgggaacccagcatggtttcggtgtccagcgggtcgccctgccggaccgccttggtccggatcgccgccagctccaggaactcgtcgtagatgtcggcctggatcagactgcgcgacgggcaggtgcacacctcgccctggttgagggcgaacatggtgaagccttccagcgccttgtcgcagaagtcgtcgtgggcggccagcacgtcggcgaagaagatgttggggctcttgccgccgagttccagggtgaccgggatcaggttgtgcgaggcgtattgcatgatcagccgccccgtggtggtttccccggtgaacgcgaccttggcgatgcggtcgctggaggccaacggcttgccggcctcggcgccgaatccgttgaccacgttgaccaccccgggcggcaacagatcaccgatcagcgacatcaggtagagcaccgaagcgggtgtctgctcggcgggtttgagcaccgccgtgttgccggccgccaacgccggcgccagcttccaggccgccatcaggatggggaagttccacggaatgatctggcccaccacgccgagcggctcgtggaagtggtaggccacggtgtcctcgtcgatctggctcagcgcgccctcctgggcgcgaatcgccgcggcgaagtaccggaagtgatcgaccgccaacgggatatcggcggccagcgcttcccggaccggtttcccgttgtcccagacctcggccaccgccagcgcggcggcgttcttgtcgatgcggtcggcaatcatgttgaggatcgccgcccgttcggccggtgcggtcttgccccaccccggcgccgccgcgtgcgcggcgtcgagcgccttgtcgatgtcggccgcgtcggagcgcggcacctcgcagaacggctggccggtcaccggcgtcgggttctcgaagtagcgcccatggaccggcgcgacccactggcccccgatgaagttt

SEQ ID NO: 70 - DNA, 462 bp, Mycobacterium tuberculosis
gtgtatcttccgcccaagctgatcccgaggcggatcccggcgcaggtgaggccaactatggtggccccccaagttccccacgtcttgtcgatcacaccgaatgggcgcagtggggaagtctgcccagcctccgggtctacccgtcccaagttgggcgtacagcctcccgccgcctcgggatggccgctgccgacgcggcctgggccgaggttctcgcgctgtcaccggaggccgacactgccggcatgcgcgcgcagttcatctgccactggcagtacgccgaaatcagacaacccggcaaacccagctggaacctcgagccgtggcggccggtcgtcgacgactcggagatgttggcttccggctgcaatccgggcagccctgaagagtcgttttagtgctcggccaaccgactcgggcgcagttggccgcgctggtagaccacaccctgctcaagcctga

SEQ ID NO: 71 - DNA, 237 bp, Mycobacterium tuberculosis
atgacgtctacgaacgggccatcggcgcgggataccggttttgttgagggccagcaggccaagacacaacttctcaccgtggccgaagtggcggccctgatgcgggtgtccaagatgacggtgtaccggctggtgcacaatggcgaactgcccgcggttcgggtcgggcggtcattccgggtgcatgccaaggccgtccacgacatgttggagacttcgtacttcgacgcgggctag

SEQ ID NO: 72 - DNA, 399 bp, Mycobacterium tuberculosis
gtggcggagtccgtggctatccgcggctgcctgctgaggtgcgggccgcgttcccgaccgcggcggagatcgcgccgcagtggcatctgcgcatgcaggccgcggtgcagcgccacgtcgaggccgccgtgtccaagacggtcaacttgcccgccacggcgacggtcgatgacgtccgcgccatctatgtggccgcctggaaggcaaaggtcaagggcatcacggtgtatcgctacggcagccgggaaggacaggtactgtcctacgccgcgccgaaaccgctactggcgcaggctgacacggagttcagcggcggctgtgcgggccgctcctgcgagttctgacggcggctcccatggcgcgagcagacgcagaatcgcacaaaatcagcgattttga

SEQ ID NO: 73 - DNA, 255 bp, Mycobacterium tuberculosis
ttgctgcacagcagcttcgggcacctcgagggcatccagcagccgctcatagacgagctggcagaactcgaccacgtgttgggcaagctgccggacgcctaccggatcatcggccgcgccggcggcatatacggtgacttcttcaacttctatctgtgtgacatctcactgaaagtcaacggattacagcctggaggtccggtacgcaccgtcaagttgttcggccagccgaccggcaggtgcacaccgcaatga

SEQ ID NO: 74 - DNA, 882 bp, Mycobacterium tuberculosis
ttgctgggggcgctgcaccagtacccgcacactcgcatccagccgggtgccgttgcggcgcaccgtgatcgccagcacccgcgcccggtctttggcgatgaggcgctcgatgcggcgggtgttctcatgcgtacgcacgcagccgatcaccggcaaagtgaggtgtctacggtcgggctcaacgcgcatcgcacccgtggtgaacgacacgcgatcggcgtcgcggcccttcttcttgaatcgagggaagcccattctcttgccgtcgcgcttgccagcacgcctctgctgccagttccagtacgcgtcgaccgcgcccgcgatcccgtcggcgtaggcctctttcgagcattccggccaccacacggtgccagtctcggcgttgacacacacctcgtctttcaccgtgttccagcgtttccgcagtacccgaagcgacggcttcgccgtctgggcgccggtcgcgcgccacgcttggatatcggctttcagctgcgcgacggtccagttgtaggccttgcggcgggcgccgaaatgccgcgccaacgcgtgtgcctgctcggcggtcggatcgagtgtgaaccggaacgcttgcacacaccagccgttggggatctccaaacgcggcatctcaggccgcctcatgatcatcgacagcggcagccgcgacggcccgcttggcccggttctgagcagcacgtttgccatacaaccttgcgcacatcgaggtcagaatctcggtcatatcccataccaggtcatcgtcaacctcggccgagtccaccacgaccaactcccgaccctgagcggccagcgcagcgtggacatactccgaaccgaaccggcagaaccgatcccgatgctcaaccacaatccgcgtga

SEQ ID NO: 75 - DNA, 168 bp, Mycobacterium tuberculosis
atggcttccagtaccgacgtgcggccgaagatcactttggcatgcgaggtgtgcaagcaccgtaactacatcaccaaaaagaaccgccgcaacgacccggaccggctggagctgaagaagttctgcccgaattgcggcaaacaccaggcgcaccgcgagacgcggtaa

SEQ ID NO: 76 - DNA, 642 bp, Mycobacterium tuberculosis
ttggtatgcgccgccgcccccggtcgacgacgacccctcggcgtaggcggacaggtcgaagccggcacagaatccctcgccgcgaccggacaccagaatgacatgcacgcctggatccagatcggcacgctccaccagagcagacaactccagcggggtgtctgcgatgatcgcgttgcccttctccggccggttgaaggtgatccgcgcaatccgaccggtgacctcataggtcatcgtcttcaggttgtcgaaatcgaccggcctgatcgcgtgtgtcatcagcggccgctcagccttttaccagcgcacgctcgaggatgggcgcgagatccagaccggccggcatggtgccgtacgctccgccccactggccgccgagccgagtggccagaaacgcctcggcgacggcgggatgtccgtggcgcaccaacaacgatccctgcaacgccaggcagatgtcttcggcaatcttgcgggctcgataaccgatcgtgtcaagatcgcccagctgcggacgcagcctttcgacgtggccgtccagcctggggtcctggcctgcgctgcgggccagctcgtcaaacagcacctcgacgcatgcgggccgggttgccatggcgcgcaaggtatctagcgcgctga

SEQ ID NO: 77 - DNA, 396 bp, Mycobacterium tuberculosis
ttgggtctcgttgcgccggcaggtgacggtcgcgcagcgaaaaagcgacctgcgggccgccgaggatccgatcgacgccgtcgtatgcgcctacgtggcgttgtacgcccaacgccggcccgccgatgtcacgatctatggggacttcaccaccgggtacattgtcacgccgtcgctgcccaccgacttcagaacggcaccggacgctggtcgacgggcgcgagcacgtcgatgaggtcgaccaccgtcgccagcgcagcggcacgcgggtcccgcccttcgaccagcgccgagaccaccgatccgtcgaccgcacagatcaacgtacacaccagttcgatctgtgcggagcggccggagcgctcgatggcctcggccacggcctcagcgcgctga

SEQ ID NO: 78 - DNA, 897 bp, Mycobacterium tuberculosis
atgcggtgtagggcggcgttgagctggcggttgcccgagcggctgagccgcatctggccggcggtgttgcccgaccacaccgggatgggagccactgcggcatggcaggcgaaggcggcttcgcttttgaaccgggtcactccggcggcttcgccgacgattttggctgcagtcagctccgcgcagccagggatttccagcagtgcgggggcgacctggtggactcgggcgctgatgcgctgggctagggtgttgatctcgccggtgagccggatgatgtcggtcagctcggcgcgcgcgagttcggcgaccaatcctggctgggtgtccagccaggtccgcagggcctgctggtgcttggcggcatcgagcgagcgtgctgccggtgcccgctcgggatcgagttcatggacgagccagcgcaaccggttgatcgccgacgtgcgttgggccacaaggacatctcgacggtcagtcaacaacttcaactcccgcgacgtctcgtcgtgggtggccaggggtaggtcggtttcacgcatcaccgcccgcgccaccgccagcgcatcgatcggatccgacttgccccgactgcgcgccgacttgcgggtctgggccatcagcttggtgggtacccgcaccacctgctggccggccgccagtaggtcacgctccagacgcgccgacatgttgcggcagtcctcgatgccccagatcagctcgaggccgaactgttcacgggcccacatgatggctgtggcgtgcccggccgtggtggccttgacggtcttctcaccgagttggcgacccacttcgtcggtggccacaaaggtgtggctgtacttgtgcgcatcggttccaacaacaaccatggtggttgcctctgaaccgccccggtga

SEQ ID NO: 79 - DNA, 798 bp, Mycobacterium tuberculosis
ttgcggcgccgagccgctgttcctgttggattacatcgccgtcggtcggatcgtgccggagcgactcagcgcgatcgtcgccggtatcgccgatgggtgcatgcgtgccggctgtgcgctgcttggcggcgagaccgcagaacatccgggcctgatcgagcccgatcactacgatatctctgccaccggcgtcggcgtcgtcgaggcggacaatgtgctgggtcccgaccgggtcaaacccggcgacgtcatcatcgcgatgggctcgtcgggtctgcattccaatgggtactcgctggtccgcaaggtgttgctggagatcgaccggatgaatctggccggtcatgtggaggagttcggtcgcaccttgggcgaagagttattggagccgactcgcatctacgccaaagactgtttggccttggccgccgaaacccgtgtccggacgttttgccacgtcaccggcggcgggctcgccggcaacctgcaacgggtcatcccgcatggcctcatcgccgaggtcgaccgcggcacctggacacccgcgccggtattcaccatgattgcccagcgcggccgggtcaggcgcacagagatggagaagacgttcaacatgggtgtcggcatgatcgccgtcgttgcccccgaagacacgacgcgcgccctggccgtcctgaccgcgcggcacctggactgctgggtattgggaaccgtctgcaaaggcggaaaacaaggcccgcgggcaaaactggttgggcagcacccgagattctaagaaccagacctaaccgggtctaa

SEQ ID NO: 80 - DNA, 747 bp, Mycobacterium tuberculosis
gtggtagcggtccggattgaagtcgtcggccatcgagtccaccacctggccggccatcttgagttccgcgggtttgatctccaccttctggtccagcaccgggaagtcggggtcgcggatctcatcgggccacagcaacgtgtgcaccatcatcacctctcgcttgccgaaatccttgacgcgcaacgccgccagcctggtcttgttgcgcagcgtgaaatgcacgatcgccatccggtcggtctcggcgagtgtcttagccagcagcacatacgatttcgacgacttcgaatcaggctccaaaaagtagctgcggtcgaacatcatcgggtccacgtcggcggcggggacgaactccaacacctcgatctcccggctgcgttcttcaggcaagctggcgatgtcgtcgtcggtgatcgccaccatttggccgtcgccggactcgtaggcccgggcaagatcgcggtagtcgaccacctcgccacacgcctcgcagacgcgcttgtaccggatgcgtccgttgtccttggcgtgcacctggtggaacctgatgtcgtggtctgcggtagcgctgtacaccttgaccggcacgttcaccagcccgaaggcgatcgaacccgtccaaatggctcgcatgtaagtgagtatgccttgattgtccgcgagcggaacgtcacggcgaaattccacgcgatatttgaccgtgacgttacgctcgcgacttgtgtgaccgacaggctacgttga

SEQ ID NO: 81 - DNA, 627 bp, Mycobacterium tuberculosis
ttgcgctcggcgagggtgaatccgccggcgcgcagtgcggcaagcacgccatggtacccaagcggatcggtgaccaccgccgcgctgggatggtttttggcggcggcccgcaccatcgccggcccgccgatatcaatctgctcgacgcagtcgtcgacactggcgccggattcgacggtctggctgaacggatacaagttgactacaacgagttcgaaagcctcgatcccgagttgctcgagggccgcggcgtgctcggacttgcgcaggtcagccagcagcccggcatgcactcgtgggtgcagtgtcttgacccggccatcgagcacctcgggaaagccggtcagctgctccacgggggtcaccggaatcccggtgtcggcaatggtcttggccgttgacccagtcgagatgatctcgacgccggccgcgctcaggccctgtgccaggtctaccagcccggtcttgtcgtacacgctgatcagcgcacggcggatcggccgtcttccgtcgtcggtgctcatcctatggttacctttcgtcccatcgtcgctgttcgtccgaccaccgtcacgccatgggtggccagtgcggccaccgccgctaccaacagccgtcgttcggtga

SEQ ID NO: 82 - DNA, 663 bp, Mycobacterium tuberculosis
gtgcgcgctgacccgccgacgaccgcctgcaacacgcgatgcacgcccagcgtctgtgtcccgtcgatgtgcggtacatcgaccacctcgatgccgccccgcagctgcgtcccggaaaaagtcaccttgctgcagtctttcccggggctgggggccggcagcggctgggacgtctccaccgcgatgacgacgaaccggttgccgttgccctcggcggagacggcggccatgttgccctgcaacccggtcggcagctggggcccggccgccacttgcgcacagttcgccggatcgaaactcagcccgtcgggcagtttgcgggcggaaaagaacccgggatcgatggccctgggagtgacatcggtgacggtgtattcaggtccaaagcccgacttcacttcggccaccttggcgatgtcgccggtcgaggcggtggtggagctggcccctgatgagcagccgacaagccagcacaccgatccgactgccagtaccgccttgcgcatcgtggtcaatctacccaacgcagcccctgagctgcgcaacgtcgacaccgttttgactagcagatcagcggcgaactgcggtgccagcggcggacgcaccgacccggggtcggtgatcagccgacggcctcgatcacttgccgggctacccggttga

SEQ ID NO: 83 - DNA, 717 bp, Mycobacterium tuberculosis
gtgggtactgcgcaagagcgagtccgaagccgatcaggcccggttccgcaccacgctctacgtcacctgcgaggtagtccgcatcgcggcactgctgatccagccggtgatgccggagtcggccggcaaaattttggacctgctcggccaggccccaaaccagcggtcgttcgccgccgtaggtgttcggctgacccccggcacagcgctgccgccgcccaccggggtatttccccgctaccagccgccgcaaccacccgaaggcaagtgagcggaccgcagcgacgggaaagccacctacgaagcgttgaccgcggtctgcgcgtcgcgtgggatgtcgagcgtggcgacgggataaaacccggaatcgtcgcggccgtcgcgggacaacagcatgggcggatagttcaccacatgggagccgttcggtttgtgctgttgccagtcgatcgcggcccgcagcgtgtagtggcccgcgggcaagccggacagatcaacgcgaaccgtctcggcgaccgacgccggtgtcggctggtcgctgctgcgatcgccgcgctggtcggagaccagcgtcttcaggtccaccgctgccggcagcgtccgaaccacctgtccggtggaatccaccagccggtagccgggcacccacttttcggtggcggcagcagcgccgtagttggtccaggtgaccgagatcgtcgcgaccttgcccgctag

SEQ ID NO: 84 - DNA, 717 bp, Mycobacterium tuberculosis
atgtcgatctccggaatcgagcgctggtcggctaccgagaacatccgcatctcggtgatctcgtcgccccagaactcgacccgcaccggatgttcggccgtcggggcaaagatgtccagaatcccgccgcgcacagcgaactcgccgcgccggccgaccatatccacccgggtatatgccagctcgaccagccgcgccaccacgccgtcgaagggggattcgtcgccaacggtcagcgtgaggggctccatcatgcccagctgcggcgtcatgggctgcagcagcgagcgcaccgaggtcaccactacccccagcggtgggcccagctgggcatcgtcggggtgggccagccggcgcagcgccatcaggcgagtgccgacggtgtcaacaccgggtgagagccgttcgtgcggcagtgtctcccaggacggcaacaacgccaccgcatccccgaacacaccacgcagttcggcggccaggtcgtcggcttcccgcccggtggcggtgaccaccagcaatggcccctgccgagccagcgcactggcgaccaacagccgcgcgctggccggcgcgatgagcgtcaattcgtcgggtcgacccccggcgcgctgcatgagctgttggaatgtcggcgcgctcagcgccaattcgacgagccccgcgatcggggtatctgagcaggcaggccccggtgcggtcatgatgcggccattctag

SEQ ID NO: 85 - DNA, 465 bp, Mycobacterium tuberculosis
gtgctggcgttctaccttcggccaaggccagggacgtggtgtacgagtgaaggttcctcgcgtgatccttcgggtggcagtctaggtggtcagtgctggggtgttggtggtttgctgcttggcgggttcttcggtgctggtcagtgctgctcgggctcgggtgaggacctcgaggcccaggtagcgccgtccttcgatccattcgtcgtgttgttcggcgaggacggctccgacgaggcggatgatcgaggcgcggtcggggaagatgcccacgacgtcggttcggcgtcgtacctctcggttgaggcgttcctgggggttgttggaccagatttggcgccagatctgcttggggaaggcggtgaacgccagcaggtcggtgcgggcggtgtcgaggtgctcggccaccgcggggagtttgtcggtcagagcgtcgagtacccgatcatattgggcaacaactga

SEQ ID NO: 86 - DNA, 267 bp, Mycobacterium tuberculosis
ttgacgaccgctggcataagcgggtcaaagggccggacgggaacaggcgaaccgtgcggtctgctgtctgcggcagggtttcgcgctggcgcgtcaggtgggttgacggcggcggagaggagcacagcaagagcttccagcgcaaacctgacgcgcaggtacctgacccatgccgaactgttgatgctcgccagggccacgggccggttcgaaacgctcaccttggtgctcggctactgcggcttacggcggtttacggttcggtga

SEQ ID NO: 87 - DNA, 546 bp, Mycobacterium tuberculosis
atgggtcagtgcccacgacctgtgcggcactggccgcctgccgtaattgtttgtagccgaactaaattgcggcgcgcctgcctgcgcgactaccgccgtcccgccccctccgacaagaagcccaacaagtcgtaccgggtaatgaccccaaccggcttgccttcctccaccaccatcaacgcatcccaatcacgcaacgccttgccggccgcactgaccaattcaccggcgcctatcatccgcagcggcgggctcatgtgtgccgacacggcgtcggccaacttggcgcggccctcgaacacggccgagagcagctcgcgttccgagacgctaccggcgacctcgccggccatcaccggcggctcggcgccgaccaccggcatctgcgacaccccgtactcgcgaagaatcccgatggcgtcgcgcacggtctccgacggatgggtgtgcaccagggcgggcagcgcgccggacttgcggcgcaacacatcaccgacggtggattgctcggtcgacccgtcaaggcggctgcgcaggaacccatag

SEQ ID NO: 88 - DNA, 618 bp, Mycobacterium tuberculosis
ttggcggcgatcccgagaaggtcacgctgttcggtgaatccgcgcgggaatcgtcacgaccctgctcgccaccccggcggccgcgggtctgttcgcggcggcgatcgcccagagctcaccggcgacatcggtctacgaccaggtgagggctcggcgcgtcgcggtttgcgtcctcgacaagctgggaatcgacccgtccgatgtgcacaggttcatgaagtgccgaccgcggcaatcctttccgcgtccagcgaagtgttcaacgaagtgccggttcgtaaccccggcacgctggcgttcgtcccgatcgtcgacggcgatctgctgcccgactacccggtcaagctggcgcaggagggccgctcacacccggttcccttgatcatcggcaccaacaagcacgagtcggcgctctttcggttgatgcgctcgccgctgatgccgatcaccccgcgcgatcacgtcgatgttcacccagattgccgccgaacagcccgatctgcaagtgccaaccgaggagcagatcggctccgcgtactcgcgatggcggcgcaaagcacgctcattgagtatggctaccgacgtcggcttccggatgccgtcggtgtggctcgctga

SEQ ID NO: 89 - DNA, 438 bp, Mycobacterium tuberculosis
gtgctggccttgaggccccagcgtcatttcacccagagccggagcgcccggcggctacgctgtgtgctcgacgatgacgtatgggtgccctgggcacggtcagggggttgcaggacagcaacacggcatttgtcggtgcgctgcatagcgggaacctgttgggggccaccggtgcggttctgcaggctccgggcaacgccgtcaacggtttcttgttcggccagacgtcgatatcgcagtcgattgacgtgtcaccggagtacggatacgagttggtcgctgtcagcgacccggttggcggaactgctggctccgctcgagccggtcacggttacgttcacgccgaccttcggtgaaccggacatggtccatctgagtggcacgaagttcgggggccttgtcccggccctcttcgaaggggtgcgcgccggcttctaa

SEQ ID NO: 90 - DNA, 861 bp, Mycobacterium tuberculosis
atgaccagctcagcaccgaagcccgcggcgtcgcgcgcatcggactggccaactacttcgccggcgccttcctgctcccctaccgcgaattccaccgtgccgcagagcagttacgctatgacatcgacctgctgggccgccggttcggagtgggcttcgaaaccgtctgccaccggctctccacactgcagcgcccgcggcagcgagggataccgttcatcttcgtccgcaccgacaaggccggaaacatctcaaagcgacagtccgcgacggcgtttcacttcagccgggtcggcggcagctgcccgctgtgggtggtccacgacgcgttcgcccagccagagaggatcgtccgccaggtggcgcaaatgcccgacggcaggtcgtacttctgggtggccaagaccaccgctgccgacgggctcgggtatctgggcccgcacaagaacttcgcggtcgggctgggctgcgacctcgcgcacgcccataaactcgtctactccaccggtgtcgtcctggacgacccgagcacggaggtcccgatcggggcgggctgcaagatctgcaaccgaacgtcgtgcgcccaacgtgcgttcccctatctcggtggtcgcgtcgcggtcgacgagaacgcgggcagcagcttgccttattcgtcgaccgagcaatcggtttgaccgcccgacgccacagcagacaacgaaaccccttatattactgtggtttcagcaggctctgggcaagcattgttgtcggtgcctgcacatagcattcagtcatgtgttccactcgggaggagatcacggaggccttcgcgtcattggctaccgcgctgtcccgcgtgctggggctgacctttga

SEQ ID NO: 91 - DNA, 243 bp, Mycobacterium tuberculosis
atgcagcttggcaatcaaaacactatgagattcgcagggcggcctcagcgttttcgccaaagcgcttaccccctgttcaaccccaacagcgcgatcgcgcttggccacccattcggcggctcgggggcacggttgatgactacagtgctacaccacatgccggacaagggaattcgctacggcttacagacgatgtgcgagggccgcggccaagccaatgccaccattgtggagttgctgtga

SEQ ID NO: 92 - DNA, 306 bp, Mycobacterium tuberculosis
gtgacggtataccgtcgaggtatggctgtgttaacggatgagcaggtcgacgccgcactgcacgacctcaacggctggcagcgcgccggtggtgtcctgcgtaggtcaatcaagtttccgacgtttatggccggtatcgacgccgtacgccgggtggccgagcgagccgaggaggtaaatcatcatccggacatcgatatccgttggcgaacagtaactttcgcgctggttacgcatgcggtaggtggtatcacggaaaacgacattgcgatggcgcacgatatcgacgcaatgtttggggcctaa

SEQ ID NO: 93 - DNA, 312 bp, Mycobacterium tuberculosis
gtgggtgcagtacggcttcaacctcaccgcatgggcggtgggatggctgccctacatcggcatactggcaccgcagatcaacttcttctattacctcggcgagcccatcgtgcaggcagtcctgttcaatgcgatcgacttcgtggacgggacagtcactttcagccaggcactaaccaatatcgaaacggccaccgcggcatcgatcaaccaattcatcaacaccgagatcaactggatacgcggcttcctgccgccgttgccgccaatcagcccgccgggattcccgtctttgccctaacttcggactag

SEQ ID NO: 94 - DNA, 708 bp, Mycobacterium tuberculosis
atgccttcgccggtgagcagcggaccgaccagccatggcacaaacaaggggtgcgggttgatcaggtctgagtcgatgaacaccacgatgtcgccgctggtggccgccagtgaacgccacaatgcctcacctttgccgggccgtaccggcacctcgggcaacgcctgttcacggctgacaacccgggcgccggaggcgatggcccggatctcggtgtcgtcggtggaaccggagtccagcacgatcaattcatcgaccaggccatcgaccagcggagagatgctgtcgatcaccgattcgatggtcgcttcctcgttgagggccggcagcaccaccgaaatcgtccgtccggcctttgccgcttccaactccccgatcgtccagccgggacggtgccaagtagtgtccaagggcagcgcgccaggggccctgccaccggcgagatcgccggcgaccagctccgatgctgtcatgcgagtcctctcaccgtgcgcgtcggcggccggaccccctgaatcgatgccaccatttccagcacccgccgggtggcggcgacctcatgcacccgaaacatgcgcgccccggcggccgcagccaacgcggtggctgccagcgttccctcaagccgttcggtcaaatccacgcccagagtctccccgacaacgtccttgttgctcaaagccatcagcacgggccacccggtcataa

SEQ ID NO: 95 - DNA, 369 bp, Mycobacterium tuberculosis
atgctttcagcggttatcctgaccgaacgtggctatccagcggtgcccctggcgggacaactggtgcaccagaggttcgtccgtcccggtcctctcgtactagggacaggtttcctcaagtttctgacgcgcgcggcggatagagaccgaactgtctcacgacgttctaaacccagctcgcgtgccgctttaatgggcgaacagcccaacccttgggacctgctccagccccaggatgcgacgagccgacatcgaggtgccaaaccatcccgtcgatatggactcttggggaagatcagcctgttatccccggggtaccttttatccgttgagcgacaccccttccactcgggggtgccggatcactaa

SEQ ID NO: 96 - DNA, 1024 bp, Mycobacterium tuberculosis
ttggtgggacgcagccgcgtactcgtcctgttcggagcgggtgaacatgtcgacgtcgttgcgttgctcggtgagcgcgcccatcggctgatcggtgaacacgtcgtgcagaccgtcgtaggccatgtggtccaaaaccgtaacgtcgccgtacttgtaacccgaccggctattcatcaacaggtggggcgccttcgtcatcgactcctgaccgccggccaccaccacgtcgaactctctggcccgaatgagttgatcagccagcgcgattgcgtcgatgccggacaggcacatcttgttgatcgtcagcgcagggacatcccaaccgatgccggccgccactgccgcctgccgtgcgggcatttgcccggcacccgcggtcaacacctggcccatgatcacgtactcgaccaaggacgccggcacgttggccttctccagggcgcccttaatggcgatggcacccagctcgctggcgctgaaatccttcagggagcccatcaacttgccgatgggtgtacgcgcgccagcaacaatcaccgatgtcgttatgactacctcctcagcgcacccgaaagccgatctgaccgacccggagaagcagattctttcccttcaggttaccgttgtgtgatgacgaccgatcaagtccacgcccgtcacatgctggctacctcgttggtaactggactcgatcacgtcggtattgcggtcgccgacctggacgttgccatcgagtggtatcacgaccaccttggcatgatcctggtccacgaggaaatcaacgacgatcagggcatccgcgaggcactgctggcggtgccgggctccgcggcgcaaatccagttgatggccccgctcgacgaatcctcggtgatagcgaagttcctggacaagcgcgggccaggcatccaacagctggcgtgccgggtcagcgatcttgacgccatgtgtcggcggctgcgctcccagggcgtccggctggtctacgagacggccaggcgtggcaccgcgaactcacgga

SEQ ID NO: 97 - DNA, 1024 bp, Mycobacterium tuberculosis
ttgcgcgcggcaacaaagtcgccatcctcgagctgctggcgcgcctgtgccaccgctggatcgacttcggtggactcctcggaactcgctgcgcccttgagctttccggctgtcgcagacaacagggaatccacccagcgactcagttggtccgcgggctggaggccctggaagctcgagatcggctgtcccgcagccaaggccaccacggtcggaaccgcttggacgccgaatatctgtgccaccctgggtgcgacgtcaacgttaaccgacgccagcgaccacttgcccttagcggcagcggccaagccggacagcgtgtcaagcaagtcgacgcatacctcgctgcggggtgaccacagcaacaccaccaccggcacttcgtcggaccggacgatcacctcgtcctcgaagttcgcctcggtgatctcggtcacaccggacggcgtcgacagtgcccggtcggcatccgtgctcgccgcagcgttttgctgggcacgttgtttgatgccggagaggtcaacagcaccggccatggccggcccgagcgggggtcgcggacgcgtcacgccgtcaagtctgtcatgccgctgcggtcatcgatccacccggtggcgccgaccctgcggcaggagccgacataccgcgatcggttggtatgaccaagatcacactggccgccaccgacccctcaaccgctatccggcccgcaatatcagtgcgtcgccctgcccgccagccccgcacaatgcggcaaccccgacgcccgatccccggcgtgccaactgcagcgccgcatgtagcgtgattcgcgtccctgacatgccgaggggatgcccgacggcaatcgcaccaccgttgacgttgacgatctgggggttcagcccgagttcgcgtatcgaggccaatgccaccgcagcgaacgcctcgttgatctccaccacgtcgagctggtccaccgagatgccctcgcgatccagcgccttgttgatcgcgttggccggctgcgattgcagtgtggaat

SEQ ID NO: 98 - DNA, 735 bp, Mycobacterium tuberculosis
gtgcggtcacggcgtctagcacccacccggccacggtcgcggcggacagccagcccagccacagccacgcgcgctgcggcgcctccccgaacaacgccgccatcagcggcaccagcaacacggtgcccaccgctcgcgcgacaacggaacaaaacgcgagcagcgcaaagccgattagcctggcgcggtggtcgttcggaacaagggctatccaggtgcggatcatcgggtgccgtcctgcgctgcggcgaccgccacccggctgccctggccggtgtcccacagccggcagtagcgtccgcccgcggcaagcaactcctcgtgggtgccgcgttcgacgatccgaccatgatcgagcacgacgatctggtcggcccgggtgatggtatgcagtcgatgggcgattaccagcacggtgcggtcccgggtcagccggttaagcgcctgttgcacaaggtattccgattccggatcggcaaacgcggtggcctcgtcgaggatgaggaccggagtgtcgccgaggatggcacgggcaatggtgagccgctgtcgctccccgcccgaaagaccactgttggctccgagcacggtatcgtagccgtccggcagccgaagcacccggtcgtggatttgcgcttcgcgggccgcgacctggacctgttcggcgggggcatccggtaccgccagcgcgatgttttcggcggcggtgccatgcacaagctgggcttcctgtag

SEQ ID NO: 99 - DNA, 735 bp, Mycobacterium tuberculosis
gtgagcgcggtattggctttgtctgctgcggtatcggcacgccgcgcaaaggctgcggaggcccacagcgcccccagcagcaacggcacgccggccagtgcagccacgccgagctgccaggagatcggcaacagggccagcgcgatcactgccggcagcaggatcgcgctggtcaacggtgtcaccagattaaccaccaggccaacaagttccggcccggtggccgcgatcgcctgccgtgccgtcgcggtgttttcggcggtaaaccaatccaaccggacaaccggaagccggtccgccacatcatgttgggtgtggttaaggacggcgaaacccagctcgataccgatgcgtgcggtcacggcgtctagcacccacccggccacggtcgcggcggacagccagcccagccacagccacgcgcgctgcggcgcctccccgaacaacgccgccatcagcggcaccagcaacacggtgcccaccgctcgcgcgacaacggaacaaaacgcgagcagcgcaaagccgattagcctggcgcggtggtcgttcggaacaagggctatccaggtgcggatcatcgggtgccgtcctgcgctgcggcgaccgccacccggctgccctggccggtgtcccacagccggcagtagcgtccgcccgcggcaagcaactcctcgtgggtgccgcgttcgacgatccgaccatgatcgagcacgacgatctggtcggcccgggtga

SEQ ID NO: 100 - DNA, 324 bp, Mycobacterium tuberculosis
atgccatcggtcattcgcgacccagatcccggtgcagcgcccgcaccgacagttgctgatcggagcgcagaagtcccatcagtgcttcagcgatcgcgacgctgcgatgcttaccaccggtacagccgatggcgattgtcatatagcgcttcccctctcggcggtagccgtcgacaaccagggatagcaaccgatggtaggactcgaggaactcagccgcgcccggccggtgcagcacatagtcgcgcacggccggatgttggccggtcagtggccgcaactcgtccacccagtgcgggttcggcaggaaccgcacgtccatga

SEQ ID NO: 101 - DNA, 957 bp, Mycobacterium tuberculosis
gtgctacggcccatacgggcgggccaacctggccgacatctggcgccgccgcgacctgccacgcgacgccaaggcaccggtgctggtacaggtgcccggcggcgcctgggtactggggtggcgccgcccgcaggcgtatccgttgatgagccatctggctgcgcgcggctgggtatgcgtgtcgctgaactaccgggtgtcgccgcgccacacctggcccgaccacattgtcgacgtgaagcgcgcgctggcgtgggtcaaggaaaacatcgccgcctacggcggggatccgaatttcgttgccatcagcggcggttcggccggcggccatctgtgcgccctggcggcgttgacccccaacgatccgcgatttcagcccgggttcgaacaggtcgacacctcggtggcggcagcggttccggtatacgggcgttacgactggtttacgaccgatgcgccggggcgtcgggaattcgtcgggttgctcgaaacgttcgtggtgaaacggaaattcagcacgcaccgcgacatcttcgtcgatgcctcaccgatccaccatgtgcgggccgacgccccaccgttcttcgttctgcacggccgccacgactccctgatccccgtggccgaagcccatgcgttcgtcgaggaactgcgggcggtgtcgaagtcgcccgtcgcctacgcggacctgccccacgcccaacacgccttcgacgtcttcggctccccgcgggcgcatcacaccgccgaggccgtggcccgcttcctgtcttgggtgtacgcgaccaacccgccggccacgtagtcagctataggccagctattgctattccgcggcacgctccagctcggccagtgccggttcgatggcatcggccatctcgtcgatgtcgttggccacctcgggtgtggtcaccaggccgaaatccagataatcctggtaggagaagcaggtga

SEQ ID NO: 102 - DNA, 888 bp, Mycobacterium tuberculosis
atgacggccagcaggcgctcggaccacacggacgcgacgcgtcgagccctcgtcgacgctggccgttacctattcgcgcggcgcgactatggtgacgtctcgatcgaagacatcgtcacccgtgcccgagtcacccgtggcgccctggactaccacttcgacagcaagaaagatctgttccagacggtactcgaggttgtcgaagccgacctggtcgccgacgtcgaagccgccatagcgaaggtcaccgacgcctggatctgctggtcgtcggcttccacgccttccttgacgcggcgaccaaaccggatgcgctgcaggtcattgcgattgacggcccgtcagtgctcgggtggggcgaatggcgccggatcgacatgcgctagggcttggtctgctggtcggggctctcgaacgcgggatggccgccggggtgattcagcgcgtaccgttgccaccactttcgcatctgctgctggccgcgctaaccgaatccgcgctgcagatcgcggacgcgacggacaaagaccggaccagagtcgaggtcgaacgcgcatttatggccctactcgaaggtctacgggtgtagcacgcccgcgatccgctacggcaacggaccaccggccgcaatcgcggccagcgtcgcgaaatgctccccgtccagcgacgccccgccgaccaggccaccatcgacgtcatcctgggccacgatgtcgccgacgtttttggcgttcaccgagccgccgtagagcacccgcaccgtatcggcaatcctcggcgaggccaacgaggccaactcttttcggatcgccgcacacacctcctgggcgtcggcggcgctggccacccgcccggtgccgatcgcccagaccggttcgtaggcgatga

SEQ ID NO: 103 - DNA, 768 bp, Mycobacterium tuberculosis
gtgcggttacgctcggaaagcgcgggcctcgcccacgcggcggatgatgtcagcggggtggtcctcggcgacgacccggaccacgatccacccgtagcggtgctggactttctcgtgccggaggatgtctttccggtagtggtagcgactggtcagatggtggtcgccgtcatactcggccgcgaccttgatgtcttgccagcccatatccaaatgggcttccgcccagccccattcgttgcgcaccgcgatctgcgtctgggggcgcggaaagccggcgcggatcaacaacaagcgcagccaggtttccttgggggactgggcaccgccgtcgacgaggtccagagcggctcttgcggccttcatgccacggcggccccgatagcgctcgatcagcggctcgacgtcggccaccttcaaatcggtggcctgtatcagggcgtcgacggccgcgacggcggggtccaatggaaatcgactggtcaggtcgagcgccgttcgctccggtgtggtcacgcgcatgccctcgatgacgcagatctcgtcgggctcgatgcgctcttcccagacttgcagccccggggcacggcggcggttggtgtcgatgatcgcggcgggaagatccgcgtcgatccacttggcgccatggaaggcagaagccgagtagccggccagcacgccgcggcggcgcgagcgcagccacagcgcttttgcacgcaattgcgcggtcagttccacaccctgcggcacgtacacgtctttatgtag

SEQ ID NO: 104 - DNA, 1024 bp, Mycobacterium tuberculosis
ttgggtgtgcgcgccgccgtcggcgtagatgatgtcacccgtggtcgccggcagccagtcagacagcagcgcgcacaccgtcttggcgaccggcgtcgcatccttcatgttccagccgatcggagcgcgctgatcccagccctcctcgagcagctggatctgggcgccggcctcctcgccgagcgcaccgccgacgatcgcactcatcgccagcgtccggatagggcctgcggcaacgagattcgaacgcacaccgtacttgccggcctcgcgcgccacgaacctgttgaccgactccaacgcgctcttggcgaccgtcatccagttgtaggccggcatcgcccggctcgggtcgaagtccatgccgacgatggaacctccggggttcatgatcggcagcagcgccttggccatcgaagcatacgaatacgccgagatgtggatgcccttggacacatccgcgtagggcgcgtcgaagaacgggttgatgcccatcccggtctgcggcatgaacccaatcgaatgcaccaccccgtcgagcttgttgcccgccccgatcgcctcggtcacccggccggccaagctggccaggtgctcctcgttttgcacgtcgagttcgagcagcggggcctttgccggcagccggtcggtgatgcgctgaatcagccgcagccggtcgaacccggtgagcaccagctgggcgccctgctcctgggctacccgtgcgatgtgaaacgcgatcgacgagtcggtgatgattccgctaaccagaatccgtttgccgtccagcagtcctgtcatgtgcgtccttgtgttgtgtcagtggcccatacccatgccgccgtcgaccgggatgaccgcaccggagatatagctcgcatcctcggaagccaggaagctgaccaccccggcgacctcggcgggggtgccgacccgcttcgctgggataaattgcagcgccccctgctgaatccgctcatccagcgcgcgggtcatatcggtgtcgatgtagcccggggccacc

SEQ ID NO: 105 - DNA, 678 bp, Mycobacterium tuberculosis
atggtgccgagcatgagggtgcgctcggattgggagccgatcgcccagagccgctcccggctcgcggtcacggcaccgcgcaacacctccgggggtcgcttcatctggattctcctcggttctgcgcgaaacggtagcagagcgccatggttgccaacgcggtcgccgggcagtctagaccggatcttcctcgtggcaaccgacaacaggacgtcgttgccgaaagggcgctgggcaccgacatctaggatgaacccacagccacgccccgacgttatgccatggcgaagagcgaccggcaggagcgggaacccagtgaagcgagcgctcatcaccggaatcacaggaccggacggctcgtatctcgctaagctcccgctgaagggatatgtggccgctggtagcccggccgaggtctatttctgctgggcgacacggaattatcgcgaattgtatgggttgctcgcggtcaacagcatctggttcaatcacgaatcaccgcgtcacggcgagacattcatgactcgtaatcctgcaccatatcgcggtcggcaacgaggcgctgatcgatgcgcagacgctgatgcgccggcccacccggataggtatcagtattggggcgttccggccagcgtacgaggcgtgatcgaccgcgcaatgggtgtttgcgttgagtaa

SEQ ID NO: 106 - DNA, 798 bp, Mycobacterium tuberculosis
ttgagcggtcagccatcggctttgcgccgacctacggtgtccccgtcggcgtgtcgccgacctacggtgtcgaagtcaaagccaaagatcgacaggatgaccagcaggatggcgccaccgactaccgacggatcggcgacattgaacaccggccaccagccgaccgacaagaaatcgacgacgtgcccgcgcagcggccccggtgcccgaaagaagcgatcaaccaggttgcccatggcaccgcccaggatcatcccaagacccagcgcccaccacggcgataccagccgccgccccatccagaaaattccgaccacgacacccgtcgcaatcagcgtcaaaacccaggtgtatccggtcgccatcgagaaggccgccccagaattacgcaccagagtccaggtcaccgtgtcgccgataatcgacaccggctggccgggcggcaacagttggacagctaccaccttggtgacaatgtcgagtgtgagcaccaccacagcgaccgacagcagcatgcgcagccgtcgcggcggcgcgggagcgttaggttcccccgcccccccggcttcctcggtcgaggtcagcggatcagccgatcctgttggttcgtcaggcacaccatcatcatcccctagggccgatatggcccgcccagaccccgcggccggatgggagcaaaccacgtgcgcaatgatcccatcatggcccgcctcaccgtcatcactactggagggacaatctcgaccaccgccggccccgatggggtgctacggccaacccattgcggggcgacgctga

SEQ ID NO: 107 - DNA, 780 bp, Mycobacterium tuberculosis
gtgcccccgaataggccggaacgccggttagggaaacctctaacagcgccgcttcgacgcgcaccagcacatccccttcgcgacggtcccggatcggtcggaaacccaccgaaaacgagtcgacgacaccagcttttacgttcgccaaagcctcgtcgccgtccggggtgtccgcaatctcgaacgccccgaacaagccgtgaggctcctcccgcaactcaacggcccggcccaccgggtagcgggttcgagcgtcgtgagagaccagcagcttcaatttgtggccgcgctcggcgatggagcgccgaaaagcgccaggagcgaacatttcctggaactcgccgtcgaagtcgcggacggtggtcgcctcgttgtagggcacgatggtgccgtgcacggttcggccttcgccagaccgcagctcggccatgcggaaaaggatgctactcaaaattcggccaccacctagcagacgcaagaaacgcgcggaatcgcttgtggcgcatggcggccgctatccgggttccagccgccccgcggcgactgcccggcgtcagcggatgccgagatgccaaactcgattgtatcacacacaaaaggtcatcaccggtccggggcaaacgggttgagcccgtcgccgtcgtcgcccggcgccaccgccagtcgctgctcggcggccggggtcaggccaaactcggaggccaagcgcagcagatgcatgcgcgccgtctccgcaaccgtcaccgccgggttccggtgcacgacaccggatttcggtga

SEQ ID NO: 108 - DNA, 456 bp, Mycobacterium tuberculosis
ttgtggaaatggaagccgcgcttggcattccaccgggcaacctggcggcgacgctggaccgctacaacgcctacgccgcgcgcggcgcagatcccgatttccacaagcagccggaattccttgcagcacaagacaacgggccgtggggggcgttcgacatgtcgctgggcaaggcgatgtatgccggattcactctgggcgggctggccacgtcggtggacggtcaagtactgcgcgacgacggcgcggtggtggccggcctgtacgcggtcggggcatgcgcgtccaatatcgcccaggacggcaagggatatgccagcgggacccagctgggtgaggggtcgtttttcgggcgtcgcgccggagcgcatgcggcagcccgagcgcagggcatgtaagcctcctcgcgccgcgactgggaatcctgcgacgcgacacgccgacaaggcgtcgtga

SEQ ID NO: 109 - DNA, 933 bp, Mycobacterium tuberculosis
ttgtggccccgtatttccgcggcgccgtcgaatcggcgatcgacagttggcggcgtgtggtgtcgacggcggcccaactgggtatcccgaccccgggattctcgtcggccctgtcgtattacgacgcgctgcgcaccgcgcggctgcccgctgcactcacccaggcccagcgcgacttcttcggcgcacacacctacggccggatcgacgaaccaggcaagttccacacactatggagttcagaccgcaccgaagtaccggtgtagcgggctagaactaaaagggggtaaaggggtaagtgatgagatttctagacgggcacccacccgggtacgacctgacatacaacgacgtgttcatcgttccgaaccgatccgaggtcgcgtcgcgcttcgacgtcgatttgtccaccgccgacggctcgggcaccaccattccggtagtggtcgccaatatgaccgcggtagccgggcggcggatggccgagacggtcgcccgccgcggtggcatcgtaatcctgccgcaggatctgccgatcccggcggtaaagcagacggtggcgttcgtcaaaagccgggacctggtgctcgacaccccagtgacgctggcacccgacgattcggtgtccgacgccatggcgctcatccacaagcgcgcacatggcgtcgcggtggtcatcctcgagggtcgcccgatcggattggtgcgcgaatcgtcctgcctgggcgtggatcgcttcacccgggtgcgcgatatcgccgtgacggactatgtgaccgctccagcgggaaccgagccacgcaagatcttcgacctgctggagcacgccccggtcgacgttgcggtgctgaccgacgccgacggcacgttggcgggagtgctaagccgcaccggggctatccgcgccggtatctacaccccggccaccgatag

SEQ ID NO: 110 - DNA, 1024 bp, Mycobacterium tuberculosis
ttgggtatatctcccggcgatcgcggggatcgtgttcgtggcaatgccgctggtcgcgatcgccatccgggtcgattggccgcgtttctgggcgctgatcactactccgtcttctcaaacggccctgctgttgagcgtgaagaccgccgcggccagcacggtgctgtgcgtactgctgggcgtcccgatggcgctggtgctggcccgcagccgcggacgactggtgcggtcgttacgaccgctgatcctgttaccgctggtgctgccgccggtagtcgggggtatcgcgttgctctacgcgttcggccggctcggcctgatcgggcgctacctggaggcggccggcatcagcatcgcattcagtaccgcggctgtggtgctggcgcagacctttgtctcgctgccgtatctggtgatttccctagagggtgcagcccgcaccgccggagccgactacgaggtggtggcggcgacacttggggcgcggcccggcactgtctggtggcgcgtgaccctgccgttgctgctcccgggcgtggtgtccggatcagtactggcgtttgcccgctcgctcggagagtttggcgcgaccctaacctttgccggttcccggcaaggggtcacccgtacccttccgctggagatttacctgcagcgggtgaccgatccggacgcggcggtggcattgtcactgctgctcgttgtggtagcggcactggtggtgctgggtgtgggtgctcgtacgccgatcgggaccgataccaggtagccggtcatgagcaagctgcagctgcgcgcggtcgtcgccgaccggcgtttggacgtcgaattctcggtgtccgcgggcgaggtgcttgcagtgctcgggcccaacggtgcgggcaagtccaccgccctgcatgttatcgcggggctgcttcgccccgacgcgggcttggtacgtttgggggaccgggtgttgaccgacaccgaggccggggtgaatgtggcgacccacgaccgtcgagtcgggctgctgttgcaa

SEQ ID NO: 111 - DNA, 516 bp, Mycobacterium tuberculosis
ttgcccacgccggtcccagcccgaactgggacgccgtcgcgcagtgcgaatccgggggcaactgggcggccaacaccggaaacggcaaatacggcggactgcagttcaagccggccacctgggccgcattcggcggtgtcggcaacccagcagctgcctctcgggaacaacaaatcgcagttgccaatcgggttctcgccgaacagggattggacgcgtggccgacgtgcggcgccgcctctggccttccgatcgcactgtggtcgaaacccgcgcagggcatcaagcaaatcatcaacgagatcatttgggcaggcattcaggcaagtattccgcgctgacggttggcggcgtgtgcggtctatgaccaggtcgacgtatgtgtttggatcaggtcatggaaggttcggccacagttcacatggcagcgccgccggacaagatctggacattgatcgcggatgtccgcaataccggccggttctcgccggaaaccttcgaggccgagtggcttga

SEQ ID NO: 112 - DNA, 609 bp, Mycobacterium tuberculosis
atgcgccggctccgctcttcagatccacggtgccatcgccttcacgtgggagcacgacctgcacctgtattaccgccgggccaagaccaccgaggcgcttttcgggagcagcgctcgaaatcgtgcgctgctcgccgaacgcgcggggcttgtgaaagcctaggcgcccagcgcggccagcgccgcttcgtagttgggttcttgcgcgatttccggcaccaattccgtgtaggcgacgttgccgtccgcgccgatcaccacgattgcgcgggcgagcagcccggccatcggcccgtcggcgatggtcacgccgtaatcctcgccgaagctgtcccggaatgccgacgcgggcatgacgttttcggtgccctcggcgccgcagaagcgcttctgggcgaacggcagatccttcgagacacacagcacggtagcgccacttgccgccgcacgctcgtcgaaggttcgcacactcgtcgcgcacaccggtgtgtccacggatggaaagatgttcagcaacacggacttaccccggaactggtcgctgctgatcacccccagatcgcccccggtcagggtgaaggccggggccggggatccgacagcaggtag

SEQ ID NO: 113 - DNA, 918 bp, Mycobacterium tuberculosis
ttgcgcgggtccgggcggacgcagatacaagaccacgccgctgccctgagccgacatcctcgccagcgcgccgttgagttcctcgccgcagcggcacgccgtcgagccgaacacgtcgcccgtcaggcactcgatgtggacgtgcagcggcacgggcaccccggcaccgaccgcacccacgatgaccgccaaatgctcgccgaggtcgtaaacgtcacgaaagccgatgacacgcgaggcgccggcccaggtgggcagcgtcgctgccgtaaaccggaccacctggggctcgatccgccggcgatacgccaccagctccccgatcgagaccatggccagtccgtgttcgacggcgaattcgaccgactcggcgtggtgcgccatctggacgggattatcgggcgagacgatctcgcagagcgcggcggccggccgccgttccgccaggcgggccaggtcgacggccgcctcggcgggtccccgccgacccagcacaccgtcggcttgcgcctgcacgggcaccacatggcccggacgttggaaatcggcggcgacggaggtggccgaagccagtgccgcgatggtccaggcgcgatcgctcgccgagattccggtgccggtgccgcgaacgtcgaccgacacgcaatgcgtggtgtctcggtcacacatgggcggcaggtgcagtcgctcgcattcggcgcccggcagcgcgacgcgcaaataacccgaggtgtgccggaccgcaaaggcaaccagccgcggcgtcgcggcctgggcggcgaagacgagatagccatcgccattggggtcgccggtcaggaccacggcgtgaccgcccgccatcgccgtgatcgcacgacgtacccgcacatcggtcgtcttcatcgagactccaaccggcggaaccggctaccgtga

SEQ ID NO: 114 - DNA, 249 bp, Mycobacterium tuberculosis
atgaagacagctatttctctgccggatgagacgttcgatcgggtatcgcggcgtgcgagtgagctcggcatgagtcggtccgagttcttcacgaaggctgcgcagcgctacctgcacgagctggacgcccaattgctcacgggccagatcgacagggctctagagagcatccatggcaccgacgaagcggaggccctcgccgtggccaacgcataccgcgtgctagaaaccatggacgatgagtggtga

SEQ ID NO: 115 - DNA, 234 bp, Mycobacterium tuberculosis
atgtctacatccacgacgattagggtttcaacccagactcgggatcgtctggccgcccaagcccgcgaacggggaatctcgatgtcggctctgctcaccgaactggccgcccaggccgagcgccaggcaatcttccgcgccgaacgcgaggcctcgcacgccgagacgaccacccaggcagtccgcgacgaggaccgcgagtgggagggcacggtaggcgacggccttggctga

SEQ ID NO: 116 - DNA, 1024 bp, Mycobacterium tuberculosis
gtggcgaccagcacctcgccggccggtgggctgccgcaggcccgctcgcagccgacgaaatgccgatgcccggctgactccacgttcagtgaccgcgcggcgtcggcccgtacgtcggcggccgagtgcgcgcagccggggctgccggtgcaggcgctgatgttcagccagggggagttctcgtcgaacaccaggcccagcggcgccagcacccgcagcgcggcgtcggccgtcgcgtcgtcgaggtcgcagatcagcaccgatcgccacggcgtgatcaccagcggggcctcgatcgcggccaggcattccgcgacccgggcgggcaagacccccagcggcaccgcggcgcccagcgttacccggctgtcatcctggggtatccagccgacgggcgttttggtgacgggccgaacggatgggcccagctcgacaccggactgcagctcgccgatatcggctaattccgttactcgccaggcggtttcgcggatcttgacgaaacgcaacgcgacctcgatcagggtctcggcgacatcggccacccgcacgccggtgtcacgtccggtcaacagcagtcggggaccgtcggggaacacctgcacgccgacgtcggcacccaggccggacacgtcggcgcggccgtcgtcgagaccgaaccagaaccggccgcccagttccgccagccggggctcggcgcggatcgccgcgtcgagctcaccgacccatgcccgcacgtcggctagcccgccggcccggccggacagcggcgaggcgacgatattgcgcacccgctcgtgtgttgccgacggcagcagcccggctttggcgaccgcgtccgcgaccgctgccacgtcgcggatcccgcgcaactggacattgccgcgcgcggtcagttccagtgtcgcggagccgaagtcgctggcgacgctggccagcgtcgccagttgtgccgcggtgatcatcccgccgggcagccggatccgcgccagcgccccgtcggcggcctggtgcggccgcaacg

SEQ ID NO: 117 - DNA, 1017 bp, Mycobacterium tuberculosis
atgacgggccgtgtccgacagaccggcataacccgtctcgtcgtacatcagcggggccccgtccttccacagcgactgatgacagtgcatgccggacccgttgtcgccgaacagcggcttgggcatgaacgtgaccgttttgccgttctgccaggcggtgttcttgatgatgtacttgtacaactgcatgtcgtcggcggcgtgcagcagcgaattgaactggtagttgatctcggcctgtccgccgctgcccacctcgtggtggcccttctccaggatgaagccggagttgatcaggttggtcagcatcttgtcgcgcaggtcgacgtattggtcgttgggggccactgggaaatacccgcccttgtggcggaccttgtagccccggttgggactgccgtcggcctcggtcgccgcgccggtgttccaccaccccgagatggcgtccacctcgtagaaggagccgttggcgcgcgagtcgaagctcaccgaatcgaaaatgtagaactcggcctcggcgccgaagtatgcggtgtcggcgatgccagtgctgatcaggtagttctcggccttgcgggcgatgttgcgcgggtcgcgggagtacggctccagggtgaacgggtcgtgcacaaagaagttgatattcagcgtcttggccgcgcggaacgggtcgatgcgcgccgtctcgggatcgggaagaagcaacatgtcggattcgtggatcgactggaacccgcgaatcgacgagccgtcaaaggccaagccgtcgtcaaacacgctcttgtcaaaggccgaagccggaatcgtgaagtgctgcatgatgccaggcaggtcacagaaccggacgtcgacatattcgaccttctcgtccttggcaagtttgaagacgtcgtcgggcgtcttttccgtcacagaatgctcctttactgtatccgcggccgacgctatggagccgatattgcccgtcagtcaaccccgtgttgcgcagacgttactgaccgtgccgcccaccactga

SEQ ID NO: 118 - DNA, 468 bp, Mycobacterium tuberculosis
gtggcgggcgtttgcgcgctattctccggtgcttcccgctggccgtctggtgaacttcggcaccgtccacagggttcccgccggggtccgagccggctacgatgcacctttccccgacaaaacgtatcaagccggcgcccgggcgttcccacggttggtgccgacctcacccgacgatccggcggtaccggccaaccgcgcggcatgggaagccctgggccggtgggacaaaccgttccttgccatcttcggttatcgcgacccgatactcgggcaagcggacggtccgctgatcaagcacattcccggcgcggcgggtcagccgcacgcccgcatcaaggccagccacttcatccaggaggacagcggaaccgaactcgccgaacgcatgctctcctggcagcaggcaacgtaaccgcgacggctgcggacgaaggatcggcagaatggcgatggagatggcgatga

SEQ ID NO: 119 - DNA, 594 bp, Mycobacterium tuberculosis
atgaccgacaacgagtgcccggccgacagccgacggcgccatgtcctgcggctcgccctgttcgccgggattttgctggggctgttctacctggttgcggtggcacgagtcatccacgtcgacggggtccgtagcgcgatcgtggtggcgacgggtccgatcgcacccctggcgtacgttgtggtgtcggccgcactcggcgcgttgttcgtcccgggcccgatcctcgccgccggcagcggggtgctgttcgggccgctactagacacctttgtgaccctgccagctttctcggccggcgcgcaggccggaatgacgcccaggcgctgctgggtgtcgatcgcgcccatcgcctcgatgcacagatcgaacggcgcggattgtgggcggtggtcggtcagcgcttcgtccccggcatctcggatgcgctggcctcgtacaccttcggggcgttcggagttccgttgtggcagatggtcgttgggtcgttcatcgggtcggcgccacgggtgttcgtctacaccgcgctgggcgcgtcgatcaccaacctgtcgtcgccgctggtttactcggcgatcgcggtgtggtgcgtga

SEQ ID NO: 120 - DNA, 435 bp, Mycobacterium tuberculosis
ttgtgggcggtggtcggtcagcgcttcgtccccggcatctcggatgcgctggcctcgtacaccttcggggcgttcggagttccgttgtggcagatggtcgttgggtcgttcatcgggtcggcgccacgggtgttcgtctacaccgcgctgggcgcgtcgatcaccaacctgtcgtcgccgctggtttactcggcgatcgcggtgtggtgcgtgaccgccatcatcggggcgttcgccgcgcggcgttggtaccggaagtggcgtgcgcgcccgcgccggcggtgcggcctggctcagctcacgaccggtagtcagcaacgccacacgagtcaccggacaccggcgggcgtcgtcatgcccggttcactgtccgagcaccgccgtctccgtcaagaagcgccggatcgcatcgagcatcacccgcccatcgagtag

SEQ ID NO: 121 - DNA, 498 bp, Mycobacterium tuberculosis
ttgtctgcggttttaccggctcggtgcattcgcgcgctagccgatagggtctatcgccatgtccggtgccacggtgggtgcgcgcgaaatcaccatccgcggagtcgtcctgggcgcattgattaccttggtgttcaccgcggccaacgtgtacctggggctaagggttggattgacattcgccacttccataccggccgcggtgatctcgatgggcgtgctgcggttgttcgccaaccactcagtggtggagaacaatattgttcagacgatcgcgtcggcggccggcacgctgtcgtcgatcatcttcgtgttaccggcactgctcatgatcggctggtggagcgggtttccgtactggacaacggcggcggtgtgtgcactgggcgggatccttggcgtcatgtactcaattccgttgcgccgcgcactcgtcaccggatcagacctgccgtacccagaaggcgttgccggagccgaggttctcaagatcggtga

SEQ ID NO: 122 - DNA, 204 bp, Mycobacterium tuberculosis
gtgggcccgatgaacgggttcctgagttggtgggacggcgtcgagctgtggctgtccggactcccgttcgcgctgcaggcgttggcagtcatgccggtcgtgctggctttggcctatttcaccgcggcattgctggatgccctgctcggccgggtcattcagttgattcgccgcgcccgccgccccgatcaggcgcccaggtag

SEQ ID NO: 123 - DNA, 1024 bp, Mycobacterium tuberculosis
atggcggacgatgtgagcggcgcggtgtaccgggccggcacggcccacggtcggccgaccggtcgcattgaacaccgcgaccgtcaggtcgtgacgcgccgggcgactgatacgcgcgcggaactggacgggctgtccgaccatcagctcgccgaagtccagcgctcgcgcgaaaaccactacccggccggatgtctcgtcatcccgcagccgttgaaccgtcgcccggaacatcaaccggccccgccccagcgacactgggctctcgctgggggtgaccgtgaccagcgcggaggtgccaaatgccacggtgattgggtggcgatcgaccgcctcggagcgcaacgcgaccgcaagcccgtaccccgcgcccaccataccgaccgcgaccaggccggcgctgatcgaacccagtcgcggagcgtgccacgaccggcgcgccacacaccaccacagtgcgccgccgccgagggccaccacgacgcagcacaaggcacacacgttgccgatcggccacacgatcccggccgccgtcacaatccagctgaccagcgccgccgggaccaggcgtacgtccaaacgggacgcgccgaagcccatatggcgcaccggtatcagacacggaccagattgcgccgcttgtccagccgcgccggaccgatgccgtcgacgtcggcaagctggtcgacgctggtgaacctaccattgcgctgccgccacgccacaatcgctgcggcggtgaccggcccgatgccgggcagggcgtccagctgctccacggtcgcagtgttgaggtcgagcacctcagctgtcttaggagctgtcttagggcctgtcgtggctgtgcccgaggtacccgccggtcccggcgtccccgcaccgaccgagctgcccagcaccctcggctgtcccgagggcggagctagcccgaccacgatctgctcaccgtcaccaagctgccgagccatgttcagtccgacggtgtccgcgccgtctaccgctccgccggcggcctgtagcgcat

SEQ ID NO: 124 - DNA, 267 bp, Mycobacterium tuberculosis
atgaggggcactgcctacgcgaccagacgctcgatgctgcccaacacccgggcggtgtggctggccaccgtcgtgcagtgcgtgaccggcgggctgggggtgacactgattccgcagaccgcggccgccgtcgagaccacgcgaagccggctggaactcgcccgattcgtcgcccctgcccggcgcgacgaatcggtttggtgtttagctctttcggcggccgcgagaagtcctaccagcgtcttgccgggattatcggcaagctga

SEQ ID NO: 125 - DNA, 1024 bp, Mycobacterium tuberculosis
atgcgcagagtattcagcggttggacaacgttggtccgctgcagcaccgcagcgaccaccgtcacgatcagggcgatgacaaagcacgtcccggtaatccactccagcgaaccgacccggccgctgacgccgcgaaagccggtggatccggtgcgtcggtgctgcagccaactgcgtcagccgaatccgaccacactgaaaaccgcgaagagtgccagcgctaagtcggccgcggtggtcgttcgcatcagcgggtctccttcggtgcgtagcagtggtcatgaaccgttgtggcggttggctcgcagggccgcatcgatcgcggcggcggccggtgcgcagtcgccgacaccggacaccaaagttgccagcgcacccgcagcgcaggcccgccgcaatgcgcgcagtcgctcggccggcgaacctgggttgcgcggccaattcgcagcaaggaccccggcaaatacgtcgccggcgccggcggtatccactggcgttaccgttggggcgggtacctcgaacaccccgtccgcgccgacgtaccgggcaccgcgcacacccagggtgatcacgaaatgtgttggtggcgacggccagtcgtttgcctcatgctcgttggcgatcaccacgtcggcgatagcggccaagtcctgcaaggagcttcgatcctggccggctggggaggcgttgaccatgacaaccgcatcggccgactgggctgcccgcgcggctgccagcgcggttgcaacaggaatctccaactgggtcaacagtacatcgcagttggcgacggccgagggtaccggagtcagatgtgcattggcacccggcgccaccagcacggtgttctcggcgctggcatcgaccacgataatcgccgtcccgctcggtccgggcaccgtgacggtcctgtccagtccaacggcgttggcgcgcaggtgggcccgcagctgggcggcggctggatcgtcgccgaatgcaccggagaactgtacctgcgcgcctgcgcgcgctgcggccaccg

SEQ ID NO: 126 - DNA, 681 bp, Mycobacterium tuberculosis
atgagcgcttctgcgtcagccgacaaggtcgtatgcgagtgctgcgagctctgtgttcctaaacagctcgcgtcagcgattcgcaacccatacggactcgtccgtgggtggcgctgtcgcatctgtaacgagcaccaaggccagccggtcaagatggcgcaagaccacgaagaggaggtccgcatccgttggggcgagacggtggacgaactccacgctgcgctggaccgcgccgggccaaggccagggacgtggtgtacgagtgaaggttcctcgcgtgatccttcgggtggcagtctaggtggtcagtgctggggtgttggtggtttgctgcttggcgggttcttcggtgctggtcagtgctgctcgggctcgggtgaggacctcgaggcccaggtagcgccgtccttcgatccattcgtcgtgttgttcggcgaggacggctccgacgaggcggatgatcgaggcgcggtcggggaagatgcccacgacgtcggttcggcgtcgtacctctcggttgaggcgttcctgggggttgttggaccagatttggcgccagatctgcttggggaaggcggtgaacgccagcaggtcggtgcgggcggtgtcgaggtgctcggccaccgcggggagtttgtcggtcagagcgtcgagtacccgatcatattgggcaacaactga

SEQ ID NO: 127 - DNA, 1024 bp, Mycobacterium tuberculosis
gtgggatcgctcaccgtgttcaccagctcggcgaggatgtcgcgcacagcggccaacacgtcggcgcgcgcactgcacagcatgaccaccgggtcgggcgggaagagcagaatgctgaacacgatagccagcccaccaccgaccagcgcgtcgaagaggcgttcgaaaaccacactgccgttggacgcgaagaccaagaccagcaccgcggagacggcggcctggttgatgaacattaagccttgcgcgaccaacccgcgtgcgcacagcaccgcgaccgacaacgcgatgaacaccaccacacccatggcgatcggtccggaaccaagcagagcatgcacgccagcacccagcacgatccccagcgccaccccgacgatcatctgttgggcacgtcgtgcgcgcagcacgttggtcgccgacatgcacaccacagccgaaatcggcgcgaagaacgcctgcggatggttgaacacgtcatgggtgagataccacgcgaggccggcgacgaccgatgtctgggtgatcggccacagcacggtgcgcaaccgttgggcgaccgcacggccgccgcaggccgtcctgactagcagcgaagcgctcatgaacgcctatttattcacactcgggtgcgacgtcgtaaccgcaaagatctggtcatgcctgctggacccgcttgggctgggcatctattccggactccttacgttgctgagcggtaatgggcgccggcgcgtcggtgagcggatcgacgccgccgccggtcttcgggaacgcgatcacctcacggatcgagtccatcccggccagcagcgcggtggtccggtcccacccgaacgcgattccgccgtgcggcggtgcgccaaacatgaacgcctccaacaggaatccgaacttttcctccgcctcggccttgtccaggcccatcaccgcgaacacccgttcctggatatcacggcggtggatacgcaccgagccgccaccgatctcgtggccgttgcagacgatgtcgtacgcgtcggcc

SEQ ID NO: 128 - DNA, 987 bp, Mycobacterium tuberculosis
gtgatcggcgatttcgccgagatgctcggcggccaggacggcgtcgctgagttggtccaacacgtcgctgtgcacccgtttgatggcgttgatgagctcgtcgaggcggacggggtaggcggtgggtgtgggctccggcatgacgtcaacagtaggttgacgttatgcattgtgtcgaccgtgattggctgcgtagtgggttctgcagcgctgccaggccgctgcgggcagggtggcgccgatcgcggccaccaggccggcgtgggcgtcgctggtgaccagcgcgaccccggacaggccgcgggcgaccaggtcgcggaagaacgccagccagccggccccgtcctcggcggaggtgacctggatgcccaggatctctcggtagccctcggcgttgacgccggtggcgatcaaggtgtgcaccccgacgacgcggcctgcctcgcgcaccttgagcaccagggcgtcggcggcgaggaaggtatacgggccggcatcgagcgggcgggtccgaaacgcctctacggcttcgtcgagctctttggccatgatcgacacttgcgacttggaaagctttgtcacaccaagtgtttcgaccaggcgctccatccggcgagtggatactcccagcaggtagcaggtcgccaccacgctggtcagtgcgcgttcagctcgcttgcggcgctgcagcagccagtccgggaaatagctgccctggcgcagcttggggatcgcgacgtcgatggttgcggcacgggtgtcgaaatcacggtggcggtagccgttgcgctgattggaccgctcatcgctgcgttcgcggtagcccgccccgcacagggcgtcggcttcagcccccatcaaggcggcgatgaacgtcgagagcagcccgcgcagcagatccgggctcgcctgtgcgagttggtcagccagaagctgctcggtgtcgataagatgagaagaggtcattgcgtcatttccttcgattga

SEQ ID NO: 129 - DNA, 381 bp, Mycobacterium tuberculosis
ttggatgagccggcgcaccgcgctcgcccgaaagggaacggagccaatcatgacggcgctcaaccgtgctgtggcatcggcgcgtgtgggaaccgaggtgatccgcgtgcgcgggctcaccttccgctacccaaaggcggccgagccggcggtgcgtggcatggagttcaccgtcggccgcggcgaaatcttcgggcttctaggtcccagcggcgcgggcaagtccaccacccagaagcttctcatcgggctgctgcgcgaccacggcggccaggccacggtgtgggacaaagagccggccgagtggggacccgattactacgagcgcatcggggtctccttcgagctgcccaaccactaccaaaagctcaccgggtatga

SEQ ID NO: 130 - DNA, 537 bp, Mycobacterium tuberculosis
atgatccctcaaatgacggtgtcctgcccgcccccgtcgacttctgagcgcgaagagcaggcgcgggcactgtgcctgcgcctgctcaccgcgcgatcccgcacccgcgccgagttagccggccagctggccaagcgcggctaccccgaagacatcggcaaccgggtattggatcggctggccgccgttggcctggtggatgacaccgacttcgccgaacaatgggttcagtccaggcgggcgaacgcagcaaagagcaagcgcgcgttggctgccgagctgcacgccaagggcgtcgacgacgacgtgatcaccacggtgctcgggggcatcgacgccggtgccgaacgggggcgggcggaaaagctggtacgggccaggctgcggcgggaggtgctgatcgacgacggcaccgacgaagcgcgggtgagccgcaggctggtggcgatgttggcgcgccgtgggtacggccagaccttggcgtgcgaggtggttatcgccgagctggccgccgagcgggagcgccgacgcgtctaa

SEQ ID NO: 131 - DNA, 1024 bp, Mycobacterium tuberculosis
ttggtgacgactctggcgccgatcttggacagtgcatcgatgactccgaagaccgcctcctcgttgccggggatcagcgacgacgacaacacgatgagatcaccagcagtcaacgtgatgctgcgatgctccccacgcgacattcgcgacaacgccgacatcggctcgccttgggtgccggtggtgatcaacacaacttggtcgggcgccatcgtttcggcggcggcgatgtcgatgagatcggaatcagccactcgtaggaagcccagttgccttgcgacgcgcatgttgcgcaccatcgatcggccgacgaacgacactcgccggcccaatgccactgcggcatcgatgatctgctgtacccgatccacgttggaggcgaaacacgcaactatcacccgtccgtcggcaccccggatgagccggtgcagcgttgggcccacttcgctttccgatggcccgacaccggggatctcggcgttcgtcgagtcgcacagcaacaggtccacgccggtgtcgccgagccgcgacatgcccggtagatcggtgggacggccgtccggtggcaattggtcgaacttgatgtcgccggtgtgcaggatggttcccgcgccggtatacaccgcgatggccaacgcgtccggagtggaatggttgacggcgaagtactcgcactcaaacacgccgtgccgggtgctctggccctcgcggacctcgacgaacaccggtgttatgcggtactcacgacatttctctgcaaccagagccaaggtgaacttcgagccgacgaccgggatgtcgggtcgcagcttgagcagaaacggaatcgccccgatgtggtcctcgtgcccgtgggtcaacaccagcgcctcgatgtcgtcaagccggtcttcgacatggcgcatgtccggcaggatcagatcgacaccgggctcgtcgtggccaggaaacaacacaccgcagtcgataatcaacagtcggcccaggtgttcgaaaaccgtcatgttgcggccgatttcgttga

SEQ ID NO: 132 - DNA, 261 bp, Mycobacterium tuberculosis
atgtccaagagatcggatgggccgagcactggcaatgcgattcgtgctcggcatcgcatcagcgtgatgactgcgcagcgatcaacctcgcacgctacgaggacaccagtagcgtcgtcggcccagttggggccgccgtcaagcgtggagccgaccgtaagacccggcctggccgggctggtggccgtgaagcgcggaagggaagcagccgcaaggctgccgaacaaccccgagacggggtgcaagtcgcgtgaccactaa

SEQ ID NO: 133 - DNA, 477 bp, Mycobacterium tuberculosis
gtggcaacgaagaacgcggcatggccttcatctacaagctgctcgaactactcgccgaacgcgacgatcgaatcacaaaggccagatgggtgtacttcctcacgcgcatgcgtaaccccaccggtgacacagcgccttttcagcagtttgctaaccggctacaccaatggttccaagatccgacagacgccaagcaactcaagaccgcgctgcacctctacatctatcgcactcgcaaggaggagtccgaatgagcgtcatccaagacgactatgtgaaacaggccgaagtaattcgcggcctgccaaagaaaaagaacggcttcgagctgaccacaacccagctgcgggtgctactcagcctgaccgcacagctcttcgacgaggcgcagcagagcgccaaccccacgctcccgcgtcagctgaaggagaaggtccagtacctgcgggtccggttcgtctaccagtccgggcgtga

SEQ ID NO: 134 - DNA, 282 bp, Mycobacterium tuberculosis
atgtcggcgcctgacgtgcggctgaccgcctgggtgcacgggtgggtgcagggagtcggtttccgctggtggacccgctgccgagcgttggagctcggcctgaccggttacgcggccaaccacgccgacggacgcgtgctggtggtcgcccagggtccgcgcgctgcgtgccagaagctgctgcagctgctgcagggcgacacgacaccgggccgcgtcgccaaagtcgtcgccgactggtcgcagtcgacggagcagatcaccgggttcagcgagcggtaa

SEQ ID NO: 135 - DNA, 537 bp, Mycobacterium tuberculosis
atgttgcacgacgtcgtccacggcagacgatgtagtgagaatggccaccggcgacgaatcactcagtaccgaatcggaacgttcatcggtaacgccgccttgtggaaccgaaagcggcacggcgatgcgcccggcctgcaacgcgccgagaaaggcgacgacgtactcgagtccctgcggagcagagatcaccacgcggtcacccgtggaaccacaacggctcagctcctgtgccacattcagcgttcgccgatacagctgcgaccacgtcagggttatcgcaacgccgtcccagtcctgttcgtaatccataaacgtgaaggccgggtcatggggttgcagacgcgcacacgcgcgcaacgcagcgggaagggaacgcacactcatgggcatcacgttaccggccacgcttggagttgtcgcagtcgccgtcggggtgtgctcgcgctccgcggtcttagccaagtcgcatctggccagctcagcaggggtttgccggctcgccatgggtccaccatcggacacggtcggatgtga

SEQ ID NO: 136 - DNA, 531 bp, Mycobacterium tuberculosis
atgcccaccaccaaagccacccagcgccgtgatgtttccaccgagatcgcttacctgacaagagcattgaaagctcccaccctgcgtgagtcagtgtcccggctggccgatcgcgcccgcgccgagaactggagccacgaagaatacctggccgcctgcctgcagcgggaagtgtcagcccgggagtcccatggtggtgagggccgcatccgcgccgcccgcttcccggctcggaagtcgttggaagagttcgactttgagcatgctcgtggcctcaaacgcgacaccatcgcacatctgggcaccctggatttcatcaccgcccgcgataacgtcgtgtttttgggccccgcctggcaccgggaagactcatcttgcggtcggcctggcgatacgcgcgtgtcaggccggtcatcgggtgctgttcgccaccgccgccgaatgggtagcacggctcgccgaggctcaccacgccgggcgcatctacgccgaactcacccggctttgccgctatccgctcctggtggttga

SEQ ID NO: 137 - DNA, 471 bp, Mycobacterium tuberculosis
atgcagtgggggtaccgcccgcttgcgggggacgaagcgatgaggtgggggtaccgcccgcttgcgagggagagcggcgcacttgacccggatcatcggcggtgtcgccggaggccggcgcattgccgtcccaccacgcggaaccagacctaccaccgatcgggtgcgcgagtcgctattcaacatcgtgactgcgcggcgggatctgaccggtctggcggtgttggacctctatgcgggttccggcgccctggggctggaggcgttgtcgcggggagcggcgtccgtgctgttcgtggagtccgaccagcgcagcgcggccgtcattgcgcgcaacatcgaggccctaggtctctccggtgcgacgctgcgccggggcgcggtggcggccgtcgtggcggccgggaccacgtccccggtggatctggtgttggccgacccgccctacaacgtcgactccgccgacgttga

SEQ ID NO: 138 - DNA, 846 bp, Mycobacterium tuberculosis
ttgggtggggttgccagcactcggcaggcatccgttcgccgttggtctgccgttcaccccctggatgcctcgccggcgttgccccgtcccggtcaacgatgtgcgaccgctcgcgcggtcgcgggccctaccccgagctggcgtgcggccgtcaggtcggcgggggtgtcgacatcgcagcgcaggcccggccaggctcctgtcagctcgacagcgcccgaacggcggtgccgcgcggacgaatccggcccgaaccgcgggtgcagcgcggtgccgaacgcacacagtaccgcggtgccggtcccaagccggtcggcgacgaagctgcgccgatggtggcgtgcggccgagattgcctcggcgagttcctgtgtctgtaatgccggcaaatcgccttgcagcacaacgatgttggaggccccttcggcaaccacgcgttcggcagcggtgatggcggtgttcagtgggtcgggatcgtcttcgggtgtcgggtcggccagtacatcggcgcccagcccggccgccgcagccgccgcggcttcgtcgggggtgataacagtgatcgagcgcagtgaaccgacacccgccgcggcggtcaacgtgtcgacgagcatggccagcaccacgttctcgcgagtctgcgccgagaacaccggggccagcctggttttggccgcggccaagcgcttgacggcgatgatcaagccgatatcgccgtcgtccggtgtgccgctcatgaagtcatcctgccagcgtcgatccacgcggcacacttcgacggcattgccgccacggtcgtggccggggcccaggcacggtcccgacggcaaccgcggcgcagattag

SEQ ID NO: 139 - DNA, 837 bp, Mycobacterium tuberculosis
ttgcgcggcaggttgatccgatacgcggtgttgttgtctccgagcttgccgctacgtcccagcgcgtcggccaccggcttccagtcggcatcggtggtggtcaccgccgaacgagctttgccggcgtggccgctgcccgctccacccttggagcccgaactgcacgccgccagtatcaccgccgccgcggtggtgatcgcgacgattctcccagcatgtttggcgcccgccatgcgcgttccctccatccgttgcatccacggcgtggatggcagttcggttagccatggtctatcgggtgattatgaaaccacgatgaagctcgatcgcaccgatccgggcacggccagacgtcctcatcgacgccctgggcgcgtatctgctggccgccgcggctcttcgacccgtggaacgcatgcgcatccgcgccgcgggcatcagcgccaccgacccacatgcccgtctgccattgccactggctcgagacgaaatccggtatcttggaacaacattcaacgaccttctgcagcggctgcaagacgcgctcgagcgagaacgtcaattcgtcagcgatgcgggccacgaacttcgcacccccttagcctcctgaccaccgaactcgaactcgccctgcggcgtccacgaagcaaccccgaactgctcgccgcaatccgctcggctctcgcggaaaccaccgacaccgcgcgcaccaccggcggcaccgggcttggactggccatcgtcgacaccctcagccaacgcaaccacgccagcgtcaccgcccgaaaccgcgccgcaggcggtgccgaaatctccctccggcttgctcttggctga

SEQ ID NO: 140 - DNA, 558 bp, Mycobacterium tuberculosis
ttgcttgggctgcccgacccccgccccgtcccacgcaacccggctgcccgtcgtcgggcgacatcccggtctctatcggcggacccgagcagccgcccggctagccagtcgcggccaaggccagggacgtggtgtacgagtgaaggttcctcgcgtgatccttcgggtggcagtctaggtggtcagtgctggggtgttggtggtttgctgcttggcgggttcttcggtgctggtcagtgctgctcgggctcgggtgaggacctcgaggcccaggtagcgccgtccttcgatccattcgtcgtgttgttcggcgaggacggctccgacgaggcggatgatcgaggcgcggtcggggaagatgcccacgacgtcggttcggcgtcgtacctctcggttgaggcgttcctgggggttgttggaccagatttggcgccagatctgcttggggaaggcggtgaacgccagcaggtcggtgcgggcggtgtcgaggtgctcggccaccgcggggagtttgtcggtcagagcgtcgagtacccgatcatattgggcaacaactga

SEQ ID NO: 141 - DNA, 933 bp, Mycobacterium tuberculosis
atgattttctgggcaaccaggtactgcacgatctggttgccgccttcaccctcgtcggtgaccttctccccggcagtcttggccggtttgggcgtcgacgccagcacggtggatccggcgttggccagccccacctcgtcgctctcgacaccgatctcggccagggtcagcacggtaacttccttcttcttggcggccatgatgcctttgaaggacgggaagcgcggctcgttgatcttctcgttcacgctgatcaccgcgggcagcgtggcctcgagggtgaatacgccctcatcggtctcacgctcgccggtgatcttgccgccctcgatcgacactttgcgcaggtgggtgagctgcggcaggcccaggtactcggcgatgatggccggcaccgcaccgcccaccccgtcggtcgattcgttgcctgcgatcaccagctcggtgccctcgatggtgcccaacgcgcgcgccaaagcccacccggtttggatgacgtccgagccgtgcatgccgtcgtcctttaggtggacggccttgtcggcacccatcgacagcgccttgcggatcgcctcggtggcgcgctcggggcccgccgtcagcacggttaccgacccttcgatgccgtcggcggcctctttctcccgaatctgtagcgcttcctccacggcgcgctcgttgatctcgtccagcaccgcgtcggcggcctcgcggtccagcgtgaaatcgccgtcggtcagcttgcgctccgaccaggtatctgggacctgcttgatcaggaccacgatgttcgtcatgactgtggttcgtcctcctcgaaggcggcccgcagcgctcgactgcggaacctcggtcacacgttttgcaaccgcacagcgatattactattcggtaagttcgcgtggtgcgccctcacaccatagcgggtggtag

SEQ ID NO: 142 - DNA, 459 bp, Mycobacterium tuberculosis
ttgctctcctcctggccaaggccagggacgtggtgtacgagtgaaggttcctcgcgtgatccttcgggtggcagtctaggtggtcagtgctggggtgttggtggtttgctgcttggcgggttcttcggtgctggtcagtgctgctcgggctcgggtgaggacctcgaggcccaggtagcgccgtccttcgatccattcgtcgtgttgttcggcgaggacggctccgacgaggcggatgatcgaggcgcggtcggggaagatgcccacgacgtcggttcggcgtcgtacctctcggttgaggcgttcctgggggttgttggaccagatttggcgccagatctgcttggggaaggcggtgaacgccagcaggtcggtgcgggcggtgtcgaggtgctcggccaccgcggggagtttgtcggtcagagcgtcgagtacccgatcatattgggcaacaactga

SEQ ID NO: 143 - DNA, 648 bp, Mycobacterium tuberculosis
ttgatcagatcgatcgatcgctgggggtccgctgccgggggggcggtcggcacgcccggtgggaccgactgtaatggccgctcctcccacccagctcggtctgcggcgacgaacacatcgatctcggcccagggcgccgcgggtccctgggtcaagaatcgggggcgttccagttttccggtggcctcatgcagccgcaccgccgccgagacgacctcatcatgcctaggctccggcgcgccggcgacgaacgtgtctgcccgccaaccagacaccacgtaccggccgtcggtcgatcggacgggccgagccaggcgtacgccgtcgacgaacaacgtctcgcgcacccgggccgaccaggccgcgcgggcgttgtcggccaccatcgacaacaccacctcgccgcatcgccagccaccttcccaaccggcacccaacaggatgggttgcgcacctgccaaaccgaacgccaccaacacgtgctcgggcggcggctcgacattcacaccggtcagcctagtagagcccatcggggtgtattgggcctgtatcggtcctagtacatcaccatgtcgggctgcatctgcttggcccacgcgacgatcccaccctgcaggtgtaccgcgtcggagaaaccggctttcttga

SEQ ID NO: 144 - DNA, 897 bp, Mycobacterium tuberculosis
atgcggtgtagggcggcgttgagctggcggttgcccgagcggctgagccgcatctggccggcggtgttgcccgaccacaccgggatgggagccactgcggcatggcaggcgaaggcggcttcgcttttgaaccgggtcactccggcggcttcgccgacgattttggctgcagtcagctccgcgcagccagggatttccagcagtgcgggggcgacctggtggactcgggcgctgatgcgctgggctagggtgttgatctcgccggtgagccggatgatgtcggtcagctcggcgcgcgcgagttcggcgaccaatcctggctgggtgtccagccaggtccgcagggcctgctggtgcttggcggcatcgagcgagcgtgctgccggtgcccgctcgggatcgagttcatggacgagccagcgcaaccggttgatcgccgacgtgcgttgggccacaaggacatctcgacggtcagtcaacaacttcaactcccgcgacgtctcgtcgtgggtggccaggggtaggtcggtttcacgcagcaccgcccgcgccaccgccagcgcatcgatcggatccgacttgccccgactgcgcgccgacttgcgggtctgggccatcagcttggtgggtacccgcaccacctgctggccggccgccagtaggtcacgctccagacgcgccgacatgttgcggcagtcctcgatgccccagatcagctcgaggccgaactgttcacgggcccacatgatggctgtggcgtgcccggccgtggtggccttgacggtcttctcaccgagttggcgacccacttcgtcggtggccacaaaggtgtggctgtacttgtgcgcatcggttccaacaacaaccatggtggttgcctctgaaccgccccggtga

SEQ ID NO: 145 - DNA, 1024 bp, Mycobacterium tuberculosis
gtgccggatctcctcgagtttgcggcccttggtctccggcgcaaagcggtacacgaccacgaacgcgacgacggcgaacgtgccgaagaccgcgaaaacgcctgcgccgccgagcacacgcagcatggtgagcgagaaggcggcaacgatcgcgttggccgtcagtgtcgaggtgagcatcgggctcgatcccatcgaccgcagccgggacgggaagctctccgcggcgtacacccagaccagcgagccgaatccgaagttgaacccgatgatgaacagcagcacgccggcgaaccccaacaccagccccgtgccaccatcggagtcgttggcgaatacggtgatcagcacggcatctgcggtgatcatcgtcgcgatgccggacaacaggatcgggcgacggcccagccgatcgaccagaaacagcgaggcacacaccgccgccaagccggcgacttgcaccatcgcgggcagggcaagcatcgcgaaatagcccgcgaagcccatggcggcgaaaagtcgcggactgtagtagatgatcgcgttgatcccggtgatctggacgaggaagccgagcgcgatgacgaacagcgtggcccgcagatacggccgccgcaccatttcgccgataccgccgccgcgttcgtcgaccgcggccgccatatcggccagctcggcatcgatgtcggcctccggctggatccgccgcagcgcgctacgcgcgtcggcgatccggcccttgagcagataccagcgggcggtatcgggcatgcgccacaacaacggcaacagcagcgtggccggcgcggcggccagcccgaacatcgcgcgccagccgtgcgatccggccaacaggtagccgaccaggtaaccgacgacgatgccgctaagcgtcgccagctgatacgcggtcaccaacgacccacgcaccgccgccggcgccgactcggccacatacaccggcaccaccaccaccgacaggccgattgtcacacccagcagcagacgcgccaccaccagcatc

SEQ ID NO: 146 - DNA, 1024 bp, Mycobacterium tuberculosis
gtgtctgacgctacgacagtgttgttcgggctgccaggagcacgggttgagcgtgtcgagcgccgcagtgacgggacccgggtggtcgatgtgatcaccgatgagccgacggcggcggcgtgcccgtcgtgcgggggtggtctcgatatcagtgaaggaatacgcggttacctcaccgaaagatctaccttatggcgaagaccgcatcatggtgcgctggaacaaaattcgctggcgatgccgagaagactactgcaagctggggccgttcaccgaggccatcacccaggtacctgcccgcgtccgcagcacgctgcggctgcgtcggcagatggccaaggcgatcggggatgcggcccgctcggtgggccgaggtcgcccaggctgacgccgtgtcgtggccgacggcacatcgggcgtttgttgcctacgccgagacgggtattgaccgagccgttgcccaccccggtgctgggcgttgaccagacacggcgaggaaaacccagatgggagcgctgcgccaagactggccggtgggtacgggtcgacccgtgggataccgggttcgtcgacctggccggtgatcaggggtttatggggcagcatgaaggccgcggcggcgcggcggtgctggcatggctgcaagcgcgcacaccgcagttccgggagagcatccagtacggtggccatcgaccccgccgctgcctacgcctcggcgatccgcacgcccgggctgctgcccaacgccaagctcgtcgtcgaccacttccatgtgaccacgctggccaacgacgcgctgaccgcggtgcgccgccgggtgacctgggcgttccacgaccggcgcggccgcaagatcgacccgcagtgggccaaccgacgtcgcttgctgaccgcccgggaacgcttgtcggacaaaagcttcgccaaaatgcggaatcggatcaacgccgtcgacccccgcgcgcagattctctcggcctggatcgccaaagaggagctgcgcaccctgctgtcgaccgtgcgc

SEQ ID NO: 147 - DNA, 219 bp, Mycobacterium tuberculosis
gtgcaggcattgcccgaaagccagctgccagagctggccgtgcagatgcgtcggcggctcatagaaacagtgacggctaccggtggccatctcggcgcgggacttggcatggtagagctgaccatcgcattgcatcgggtgttcacctcgccacacgacatcggtgttcgacaccgggcaccaaacctatccgcacaagctgctcaccggccgcggtaa

SEQ ID NO: 148 - DNA, 720 bp, Mycobacterium tuberculosis
atgtcttcagaggggggttggcccaacgtcggaaacctcgcgcgcagcgcatcaatgacatcggcagtttcatcaagtgccagggttgtctgggtcagatacgatagctgggtaccctcgggcaggttcaacgctgccacatcagcgggtgtctgcaccaataatgttgaccgcggagcgacgccaagcgtgccttcggtctcctcatgtccggcgtgcccgatgaagaccaccgtgtcaccgcgcgcggcaaaccgtgcggcttcagcgtggactttcgccaccagtgggcaggtcgcgtcgacgacctgcagtccccgctcatcagcgcccgcgcgcaccgccggggaaaccccatgcgcggagaacaccacgaccgcccccggcggcggcggatcgggaatctcgtcgagatcctcgacgaacactgctccccggtcccgcaactcggcaaccacaacagtgttgtgcacgatttgcttgcgcacatacaccgggccttcggccacgtcaagcactcgcttgaccgtctcgatagcacgctctacaccggcgcaaaacgaccgcggcgacgccaacagcaccgtgacttcacccgaagcgtatccctgtgcgaccggtcccacgaacacctcagccatcagcactcccggcgacatatcagttgcgacaacgcgatcaggtctggggatcgcaccgcatcgggcagtgccgcaatag

SEQ ID NO: 149 - DNA, 522 bp, Mycobacterium tuberculosis
ttgcctgggcatcgtcggggcacgtcggcttcaagggttcccggaaatcgaccccgtttgcggcccagctggccgcggagaacgccgctcgcaaggcccaagaccacggggtgcgcaaggtcgacgtgttcgtcaagggcccgggctcgggccgcgagaccgcgatccggtcgctgcaggccgccggcctggaggtgggcgcgatctcggatgtcaccccccagccgcataacggtgtccggccccccaagcgccggcgcgtctaggagagaagatggctcgttacaccggacccgtcacccgcaaatcacggcggttgcgcaccgacctcgtcggtggcgaccaggccttcgagaagcgtccctacccgcccggccaacacggtcgcgcgcggatcaaggaaagcgaatatctgcttcagctgcaggagaagcagaaggcccgtttcacatacggcgtaatggaaaagcagttccgccgctactacgaagaggccgtgcggcagcccggcaagacgggtga

SEQ ID NO: 150 - DNA, 642 bp, Mycobacterium tuberculosis
gtgggacgccgtgatcgcggtgcacctgcgcggccattttctgctcacccgcaacgccgctgcctactggcgggacaaagccaaggatgccgaagggggatcggtcttcggccggctcgtcaacacctcgtcggaggcgggtctggtgggcccggtggggcaggcgaattacgccgccgccaaggctggcatcaccgcgctaaccctgtcggcggcgcgggcgctcgggcgctacggcgtttgcgccaatgtgatttgtccgcgggcgcgcaccgcgatgacggccgatgtcttcggcgccgcacccgatgtcgaagcgggccagatcgacccgctgtcgccgcagcatgtggtaagcctggtccagtttctggcgtccccggctgccgcggaagtcaacggtcaggtgttcatcgtctacggtccgcaggtgacgctggtgtcaccgccgcacatggagcgccggttcagcgcggacggcacgtcctgggatcccaccgagctcaccgcgacgctgcgggactactttgctggtcgggatccggaacagagcttttcggcgaccgatctgatgcgtcagtgacccgtggatataggcggccgattattggaatcggtgtccgaatcaccacgccaacatag

SEQ ID NO: 151 - DNA, 576 bp, Mycobacterium tuberculosis
ttgccttggacggcatgttgctccccttattcgaacgacaaccggaccaaacccagcccggtgaagtcggcgacaaactcgtcgccggcccgcgcctcgaccgcgaacgtgcatgacccgggtaacacgatgtcgcctttgcgcagccgcacgccgaaactctcgaccttgccggccagccaagccaccgcggtcgccgggttacccaacaccgcatcactgcggccctcggccaccacctcgccgttgcgggtcagcttcgcatcgatcgccctgacgtcaagatcggccggcggcacccgggccgcgcccaacacgaagcccgccgccgaggcgttgtcggcgatggtgtcgcagatcttgatctgccaatccttgatcctggtgtcgatcagctcgatggcgggcaccagggcctcggtggccgccagcacgtcgtcctcggtgcagcccgcacccggtaggtcggcggccaggatgaagcccacctccacctcaacccgcggagacaggtaccgggacgcctggaccggcgtgtcttcgaacacctgcatgtcgtcgagcaggtgtccgtag

SEQ ID NO: 152 - DNA, 639 bp, Mycobacterium tuberculosis
gtggttcactctcggcgctcatgggcgccatcccgccgcccgcatcgcggcatcgacgcggccaacgaacgtgccccggcggtaccagagcagctcactggtgaccctgatgatcgtccagcccagatccagcaacgcggtggaccgctcgatgtcccgagcccgctgcgccgggtctgtccaatgctgtggcccgtcatactcgacaccgactcgcaattgctcgtagcccaggtcgatgcgggcgacgaagtccccgtagtcgtcaaacactctgatctgtgtttgcggcttcggcagaccggcatcgatcaacaccaatcgggtccacgtctcctgtggggattccgcacccccgtcgatcagcggcagcaccgcacggaggcggaccaggccgcgcgcaccggtatgttcggcaatgacggcctgcacgtcggcgaccttgacatcggtcgaattcgccaacgcgtccagccgttgaacggcctgcagccgcgagggtgtgcgccgcccgatatcgaaggcggtgcgcgccggggtggttaccgcgacaccgtcaaccgcaaccgtctcgtgcggcgccaatcgatccgtgtgcacgacgatgcgcggcggaggctttcgattggcgtgcactaa

SEQ ID NO: 153 - DNA, 705 bp, Mycobacterium tuberculosis
gtgtcgcgctaccccaacagctggcgcaggttgaacaaccccgatatggcggtgcccatgttaaacaggcccgtgttcaagccgctccggacggagccaaagagggtgcccgggacgccgatgttgccaatgcccgaggtctggccgttgatgacagtgcccccgctggccgtgttgaagaacccggagacgtcgacggctaaggggccggtgggggtgttgaagaagcccgagacgtcggtgccggtgttgccgaagcccgagttggtcaggccgctgtcggtaatgatcccgaaaccggtgttcacattgcccgcattccacgagccggtgttgatgttgcccgagttcccattgccggtgttgacgttgccggagttgtcaaaccccgtgttgacgaagcccgcgtttccgaagccggtgtttaattcacccgcgttccccaagccggtgttgaggatgctcgcgttcccgaagccggtgttgagaacgcccgcgttcccgaagccgatgttggcgttgccggaattcccgacgcccaggttgttgaggtcgccaggcaccagggtattggctccggtgttgaagacgccgatgttgccgctgccggagttgaacaagccgatgttgttggtgccggagttgccgatgccgatattgccgctgccggagttcagcagcccggccaggttgatgcccatctga

SEQ ID NO: 154 - DNA, 243 bp, Mycobacterium tuberculosis
ttgagctcaaatcatgcgattctgcgtctgctcgcgcccttgcggctagatccccagaacctgggcgctggcccacagcgcgagcaccgccatcgccagggccgcaggcacggtgcacagtcccagtcgggtgtactcgccgacgctggcgtcgacgttgtgccggcgcagcacgccccgccacagcaggttagacagcgaaccggcataggtcaggttgggtccgatgttgaccccgagtag

SEQ ID NO: 155 - DNA, 345 bp, Mycobacterium tuberculosis
ttgtgccagggtgtacccgcccgattgccgccggcaaccgacactgttggtgtagtgaccaaatcagcagtgccccgggtgggtcttgacgtgcaaatcgactacagtcttggtgaccgtccggtacccgggcatgggactggaacgaaccaagaaacctgtgaggccgtctgctatggagcggttcgacggtttgcgtccggccaggctcaaggtggggatcatctcggctggccgggtcggcaccgcgctaggggtcgcgctgcagcgcgccgaccatgttgtggtggcgtgcagcgccatctctcatgcgtcccggcggcgcgcgcagcgccggctgcctga

SEQ ID NO: 156 - DNA, 603 bp, Mycobacterium tuberculosis
atgcggcccgcaaaacgggccgaggaggagccaggcaatcaccccagagccgggtgcagcgggtcgccaccatcagccccgtggcgatcgcaaaccccgcgcctggcgacaatgcggcccgcaaaacgggccgaggaggagccaggcaatcaccccagagccgggtgcagcgggtcgccaccatcagccccgtggcgatcgcaaaccccgcgcctggcgacaatgcggcccgcaaaacgggccgaggaggagccaggcaatcaccccagagccgggtgcagcgggtcgccaccatcagccccgtggcgatcgcaaaccccgcgcctggcgacaatgcggcccgcaaaacgggccgaggaggagccaggcaatcaccccagagccgggtgcagcgggtcgccaccatcagccccgtggcgatcgcaaaccccgcgcctggcgacaatgcggcccgcaaaacgggccgaggaggagccaggcaatcaccccagagccgggtgcagcgggtcgccactggctagaccaacgaccggtagttcccgacggcgtcggaaaatccgacagctgagcgttcgggtcaaacacgcggtgcaccggacctga

SEQ ID NO: 157 - DNA, 225 bp, Mycobacterium tuberculosis
atgcgcactacgatcgacctcgatgacgacatactgcgggcgttgaaacgacgccagcgcgaggagcgcaaaacgttagggcagctcgcctccgaattgcttgcgcaagctctggcggccgagcctcctccaaacgttgacatccgctggtcgactgccgacttgcggccccgtgtggatcttgacgacaaggacgctgtttgggcgattttggaccgtgggtga

SEQ ID NO: 158 - DNA, 357 bp, Mycobacterium tuberculosis
gtgtcacgttgtcggattcactgtcgccggctagcgctttcccgtcagaagacgagaagcctccccgatctccaactagcatcgagatcgggcttgcgaaggttgggttgcaaaatggatgtcatcagatgggctcgccggcttgcggtggtggcgggcacagcagcggcagtgaccactcctgggctactgagtgcgcacgttccgatggtctccgccgaaccgtgtcccgacgtcgaggtggtgtttgcccgtggcaccggggagccacctggtattggcagcgtcggaggactgttcgtcgacgcactgcgtttcccaggttggcgccaagtcactcggggtctacgccgttaa

SEQ ID NO: 159 - DNA, 414 bp, Mycobacterium tuberculosis
gtggatgcatgtcattcccgggcgcggcgcggcgtggttgatcgtcgacgtccgagatgtggcggcactgcacgcggcgttgttggaatccgggcgtgggccgcgccgctacactgcgggaggtcatcggattccggtgcccgagctcgcgaaaattctgggcgggtcgccggcaccacgatgctggccgtcccggtgcccgattccgcgctgcgtgtcgcgggatcggtgctggatcaagccgggccctatctgcctttcaatactccgttcaccgcggcaggtatgcagtactacacacagatgccggagtccgacgattcgccgagcgaaaaagaactaggcatcacctaccgcgatccgcgcgacaccgtggccgacaccgtcacggccctgcgcggcctgggcagctaa

SEQ ID NO: 160 - DNA, 732 bp, Mycobacterium tuberculosis
ttgatgtggaagccgcgctggcgatggtgttcgacggcttcggagcggcgaaccaccgccagcccagatgcctgccgcaacgtatcgcggtgccggtcaccaagcttaagacttgccggctcgggatcaccgtggcatcggatgcgatcgagatccacggcggcaatggctacatcgagacctggccggtggcccggttgctgcgtgacgcgcaagtcaacacgatctgggagggccccgacaacatcctgtgtctggatgtgcggcgcgggatcgagcagacgcgcgctcacgagacactgttggcgcggctgcgcgatgcggtgtcggtgtccgacgatgacgacaccacgcggctggtctcgcgccgcattgaggacctcgacgcggcgatcaccgcttggaccaaactcgacaggcagctggccgaggcgcggctgttcccgctggcccaattcatgggcgacgtctacgccggcgcgttgctcaccgagcaggccgcctgggaacgggcaacccgcggcaccgaccgcaaggcactcgtcgcccgcctgtacgcgcgccggtatctcgccgaccaaggcccgctgcgcggtatcgacgcagattgcgatgaggcgctgcagcgtttcgacgaactcgtggcgggcgcgttcactgccgagcagacgtaaaagcccccaattcgtggctcttctgacacttccgtgggtgagtttgtgtcctgagtag

SEQ ID NO: 161 - DNA, 594 bp, Mycobacterium tuberculosis
gtgcgggccccggcgacccgcgcggccagccgcggctcttcgaggaattccgaccagcgcccgtcgggcaggtcggtgatcccgtcgcggccttccagcagcgcctgccaggtctgctcgggggtgttcatctcgcccgggaagcgggtggacaagcccacgatcgcgatgtcgacgcgctcggccgggccggtgcgcgaccagtcttcggcgtcatcgcccgctaggtcggtctccggctcgccctcgatgatccgggtggccagcgattcgatggtcggatgcgcgaacgccaccgcgaccgacagcgtgaccccggtcaggtcttctatgtcggcggccatcgcgacggcatcgcgcgacgacagacccagctccaccatgggcaccgattcgtcgatcgagtccggtgcctttccgacggccttacccacccagttgcgcagccactggcgcatctcggggaccgttagctcggccctttcggcgggggcgttctcctgggattccgctacgtcagccatgggtcctcagtccgaagtggcgaagaccgtcggggaacccacgccactgcgcaggctgccgtcgaggtag

SEQ ID NO: 162 - DNA, 693 bp, Mycobacterium tuberculosis
ttgcacgaggacccgcacactggcgtcgagccgggtgccgttacggcgcaccgagattgccagcacccgcgcccggcctgtggcgatgagccgttcaatccggcgtgtgttctcgtgcgtacggacggtcccgacgaccggaagtgtgagatgacggcgatcaggttcgacgcgcatcgctccggtcgtgaatgtcacgcggtcctgatcgcggcctttcttcttgaaccgggggaagcccattgtcttgccctcacgtttaccggatcgggagttctgccagttccagtacgcatcgacagcgccgccaatgccgtcggcgtaagcctctttcgagcactccggccaccacaccgccccggtctcggcgttgacacacacctcgtccttgacggtgttccaccgtttacgaagcacccgcagcgacggcttgacagtcccgataccagtaacgcgccacgcctcgatatcggctttcaaagtagcgaccgcccagttgtaggccttgcggcgagcgccgaaatgccgcgccagcgcgcgggcctggtcctcggttgggtccagcgtgaaccggaacgcctgcacacaccagccttctggcacctcgaatctggccatcaagctgcctccgcgtccccgaccgcagcagcaagggcacgcttggccccgttctgtgcagcgcgttcaccatag

SEQ ID NO: 163 - DNA, 447 bp, Mycobacterium tuberculosis
ttgcgcccgtcaaggtccaccctgatagccaaatgcgccagctggcggcaaccaccccgttgtcttcgatccgcagccgtaaaccgtcgttcgtcggcgcccgtcgcccaacgtgaactgagggcggagaatcggccggaatctcgccctcagttcacgctcggcgccgtttggcctcacccagtcaatgtgatctgtgcgggcgggcgttggcgcgtagcgaaccccagtggcgccggcccgccaagcacgccccggcgcggccagctcatcagcggctacgcaagcgcaacggcgcccgcgatgggctgtggaagaacccggaggatctcaccgaacaccagaatgccaagctgtcgcgctcatctactcaaagaaggcctacggcacctgttttcggtcaaaggcgaagagagtaagcaggcactggaccggttgatcttctag

SEQ ID NO: 164 - DNA, 537 bp, Mycobacterium tuberculosis
gtgcattcggctagctcggttgccacacccgtcaggggttcgacgttggcgggttcggcgggccccagcaccgctgtcaccatgcccgccaagccgacctgcggcgccaccaactgcagcaccagcatgtcgccgtcgcgcgccgcgatcacatggcggtcgcccctgcggcacacgacgaagcgcaccatgacgccgccaatgtcgcgccgccaccagcgaccctccaaggtccgatctggcctgcccagggtttcgaccatctccgcgaccgtcggttggggctccccgtggaggtcgagcaccccttgcgctgtgaggtcacgctgcacctgttcccagacgatgtctcgcagatcctcttgcgggatattcggccgaatcccaagcgtgacagggaaatcaaccaggtgtaaccgatcggcgatcaccaacatgccgtcgatggttacctcgacgccgaccacgttgtcggcggtgcccgcgcggcctgcagcggacggacccgtcatgatcaaccgaaaatcttgtcgataa

SEQ ID NO: 165 - DNA, 462 bp, Mycobacterium tuberculosis
atggaccgactctgcggtgcgccgctatgtcaccgacgccggggccctactgccacggctgcacaagctggtgcgcgccgactgcacgacccgcaacaagcgccgggccgcgcggttgcaggccagttacgaccggctggaagagcggatcgcggagctggccgcccaggaggatctggatcgggtgcgccccgacctggacggcaaccagatcatggcggtgctcgacattccggcgggcccgcaagtcggcgaggcgtggcgctacttgaaggagctgcggctagagcgcggcccgttgtccaccgaggaggcgacaaccgagctgctgtcctggtggaaatcacgggggaaccgctagcttgggagtcgcgtcagaacggttgtggagtactgcatagccggcgacgacggcagcgccgggatctggaaccgcccgttcgacgtcgacctcgacggtga

SEQ ID NO: 166 - DNA, 525 bp, Sars coronavirus
gtgacgagcttggcactgatcccattgaagattatgaacaaaactggaacactaagcatggcagtggtgcactccgtgaactcactcgtgagctcaatggaggtgcagtcactcgctatgtcgacaacaatttctgtggcccagatgggtaccctcttgattgcatcaaagattttctcgcacgcgcgggcaagtcaatgtgcactctttccgaacaacttgattacatcgagtcgaagagaggtgtctactgctgccgtgaccatgagcatgaaattgcctggttcactgagcgctctgataagagctacgagcaccagacacccttcgaaattaagagtgccaagaaatttgacactttcaaaggggaatgcccaaagtttgtgtttcctcttaactcaaaagtcaaagtcattcaaccacgtgttgaaaagaaaaagactgagggtttcatggggcgtatacgctctgtgtaccctgttgcatctccacaggagtgtaacaatatgcacttgtctaccttga

SEQ ID NO: 167 - DNA, 207 bp, Sars coronavirus
ttggacctgagcatagtgttgcagattatcacaaccactcaaacattgaaactcgactccgcaagggaggtaggactagatgttttggaggctgtgtgtttgcctatgttggctgctataataagcgtgcctactgggttcctcgtgctagtgctgatattggctcaggccatactggcattactggtgacaatgtggagaccttga

SEQ ID NO: 168 - DNA, 186 bp, Sars coronavirus
atggtgacttcttgcattttctacctcgtgtttttagtgctgttggcaacatttgctacacaccttccaaactcattgagtatagtgattttgctacctctgcttgcgttcttgctgctgagtgtacaatttttaaggatgctatgggcaaacctgtgccatattgttatgacactaatttgctag

SEQ ID NO: 169 - DNA, 237 bp, Sars coronavirus
ttggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctag

SEQ ID NO: 170 - protein, 174 aa, Haemophilus influenzae
VTSLALIPLKIMNKTGTLSMAVVHSVNSLVSSMEVQSLAMSTTISVAQMGTLLIASKIFSHARASQCALFPNNLITSSRREVSTAAVTMSMKLPGSLSALIRATSTRHPSKLRVPRNLTLSKGNAQSLCFLLTQKSKSFNHVLKRKRLRVSWGVYALCTLLHLHRSVTICTCLP

SEQ ID NO: 171 - protein, 68 aa, Haemophilus influenzae
LDLSIVLQIITTTQTLKLDSAREVGLDVLEAVCLPMLAAIISVPTGFLVLVLILAQAILALLVTMWRP

SEQ ID NO: 172 - protein, 61 aa, Haemophilus influenzae
MVTSCIFYLVFLVLLATFATHLPNSLSIVILLPLLAFLLLSVQFLRMLWANLCHIVMTLIC

SEQ ID NO: 173 - protein, 78 aa, Haemophilus influenzae
LAPAILITMLPPCYNFLKEQHCQKASTQREAEAAVKPLLAPHHVVAVIQEIQLLAAVGEILLLEWLAEVVKLPSRYCC

SEQ ID NO: 174 - protein, 210 aa, Haemophilus influenzae
LLLKGVIMQVSRRKFFKICAGGMAGTSAAMLGFAPANVLAAPREYKLLRAFESRNTCTYCAVSCGMLLYSTGKPYNSLSSHTGTNTRSKLFHIEGDPDHPVSRGALCPKGAGSLDYVNSESRSLYPQYRAPGSDKWERISWKDAIKRIARLMKDDRDANFVEKDSNGKTVNRWATTGIMTASAMSNEAALLTQKWIRMLGMVPVCNQANT

SEQ ID NO: 175 - protein, 808 aa, Haemophilus influenzae
MTNNWVDIKNANLIIVQGGNPAEAHPVGFRWAIEAKKNGAKIIVIDPRFNRTASVADLHAPIRSGSDITFLMGVIRYLLETNQIQHEYVKHYTNASFLIDEGFKFEDGLFVGYNEEKRNYDKSKWNYQFDENGHAKRDMTLQHPRCVINILKEHVSRYTPEMVERITGVKQKLFLQICEEIGKTSVPNKTMTHLYALGFTEHSIGTQNIRSMAIIQLLLGNMGMPGGGINALRGHSNVQGTTDMGLLPMSLPGYMRLPNDKDTSYDQYINAITPKDIVPNQVNYYRHTSKFFVSMMKTFYGDNATKENGWGFDFLPKADRLYDPITHVKLMNEGKLHGWILQGFNVLNSLPNKNKTLSGMSKLKYLVVMDPLQTESSEFWRNFGESNNVNPAEIQTEVFRLPTTCFAEEEGSIVNSGRWTQWHWKGCDQPGEALPDVDILSMLREEMHELYKKEGGQGIESFEAMTWNYAQPHSPSAVELAKELNGYALEDLYDPNGNLMYKKGQLLNGFAHLRDDGTTTSGNWLYVGQWTEKGNQTANRDNSDPSGLGCTIGWGFAWPANRRVLYSRASLDINGNPWDKNRQLIKWNGKNWNWFDIADYGTQPPGSDTGPFIMSAEGVGRLFAVDKIANGPMPEHYEPVESPIDTNPFHPNVVTDPTLRIYKEDREFIGSNKEYPFVATTYRLTEHFHSWTAQSALNIIAQPQQFVEIGEKLAAEKGIQKGDMVKITSRRGYIKAVAVVTKRLKDLEIDGRVVHHIGLPIHWNMKALNGKGNRGFSTNTLTPSWGEAITQTPEYKTFLVNIEKVGEA

SEQ ID NO: 176 - protein, 65 aa, Haemophilus influenzae
VMSRHRGAKHRRRYELLGGISLLSPEYLLSVERWPFHSEPPDHYDLLSYLLDLSVSQLSLLIPLH

SEQ ID NO: 177 - protein, 59 aa, Haemophilus influenzae
VFMLYLEFLFLLLMLYIGSRYGGIGLGVVSGIGLAIEVFVFRMPVGKHRLMLCLSFLQW

SEQ ID NO: 178 - protein, 99 aa, Haemophilus influenzae
MAAAIQQRAELQRRIWQIANDVRGSVDGWDFKQYVLGTLFYRFISENFANYIEAGDESVNYAQLPDEIITQMPLKRKATLFTQANYLRMLRLMLAAILI

SEQ ID NO: 179 - protein, 273 aa, Haemophilus influenzae
LNTDLKQIFTDIENSATGFPSEQDIKGLFADFDTTSNRLGNTVKDKNDRLTAVLKGVAELDFGKFEDNHIDLFGDAYEYLISNYAANAGKSGGEFFTPQSVSKLIAQIAMHGQTSVNKIYDPAAGSGSLLLQAKKQFDEHIIEEGFFGQEINHTTYNLARMNMFLHNINYDKFDIALGNTLMEPQFGDNKPFDAIVSNPPYSVKWAGSDDPTLINDERFAPRRRACTKIQSGLCLYFTCVKLSFSKRPRGDCFLPWYFLSWRCRAKNSSIFGG

SEQ ID NO: 180 - protein, 108 aa, Haemophilus influenzae
MMNDLPPAGVLAPKSKADFAFILHALSYLSAKGRAAIVSFPGIFYRGGAEQKIRQYLVDNNYVDAVIALAPNLFFGTSIAVNILVLSKHKPNLSMPAVYLNLPLITTF

SEQ ID NO: 181 - protein, 67 aa, Haemophilus influenzae
VPHLAKSISFEEIAQNDYNLAVSSYVEQKDTREVINIDELNAQIRETVTNIDHLRAEIDKIVAEIEG

SEQ ID NO: 182 - protein, 163 aa, Haemophilus influenzae
MTQYKTIAESNNFIVLDQYNKFVEESNAGYQTERSLEREFIRDLQAQGYEYLQWLNNHDELIKNLRAQLQRLNNVVFSDAEWQRFLEEYLDKPSDNLIEKTRKIHDDYIYDFVFDNGRIQNIYLLDKKNLANNSLQVINQFKQTGSYDNRYDVTILVNGLPLY

SEQ ID NO: 183 - protein, 868 aa, Haemophilus influenzae
MVYPFIELKKRGVAIREAFNQIHRYSKESFNKENSLFKYIQIFVISNGTDTRYFANTTKRNKNSYDFTMNWATAKNTLIKDLKDFTATFLQKNTLLNVLVNYCVFDVSDTLLIMRPYQIAATERILWKIQISYLAKNWSNRESGGYIWHTTGSGKTLTSFKASRLATELDFIDKVFFVVDRKDLDYQTMKEYQRFSPDSVNGSESTAGLKRNIEKDDNKIIVTTIQKLNNLMKSEENLSIYQKQVVFIFDEAHRSQFGEAQKNLKRKFKKFYQFGFTGTPIFPENALGAETTASVFGAELHSYVITDAIRDDKVLKFKVDYNDVRPQFKALETEKDPEKLTALEQKQAFLHPERIKEISQYLLNNFKQKTHRLNATGKGFNAMFAVSSVEAAKRYYETLQNLQAEQEYPLKIATIFSFAANEEQDAIGDIPDETFEPTALNSTAKEFLTKAIDDYNHYFGTNYGVDSQSFQNYYRDLAKRVKNQEVDLLIVVGMFLTGFDAPTLNTLFVDKNLRYHGLMQAFSRTNRIYDTTKTFGNIVTFRDLEQNTIDAITLFGDKNTKNVVLEKSYDSYFNGDDNQRGYAEIVKELKESFPDPTEIETEQDKKEFVKLFGEYLRVENILQNYDEFAALQALQAVDLNDPIAMEKFKQVHYVNDEQIAEMLKVPTLPVRAEQDYRSTYNDIRDWLRQRKEGNDKDNSPINWDDVVFEVDLLKSQEINLDYILALIFEHHKKNQDKEVLIDEIRRTVRSSLGNRAKESLIVDFINQTNLDDIPDKATLIDSFFLFAQAEQRKEAESLIQEENLNVDAAKRYISTSLKREYASENGTALNEVLPKMSLLKPQYLTKKQKIFQKIAAFVEKFKGVGGKI

SEQ ID NO: 184 - protein, 347 aa, Haemophilus influenzae
MDIIKPICTGFFYNDNNVLGDLMKNFKYFAQSYVDWVIRLGRLRFSLLGVMILAVLALCTQILFSLFIVHQISWVDIFRSVTFGLLTAPFVIYFFTLLVEKLEHSRLDLSSSVNRLENEVAERIAAQKKLSQALEKLEKNSRDKSTLLATISHEFRTPLNGIVGLSQILLDDELDDLQRNYLKTINISAVSLGYIFSDIIDLEKIDASRIELNRQPTDFPALLNDIYNFASFLAKEKNLIFSLELEPNLPNWLNLDRVRLSQILWNLISNAVKFTDQGNIILKIMRNQDCYHFIVKDTGMGISPEEQKHIFEMYYQVKESRQQSAGSGIGLAISKNLAQLMGRGFNS

SEQ ID NO: 185 - protein, 65 aa, Haemophilus influenzae; identical to SEQ ID NO: 176

SEQ ID NO: 186 - protein, 653 aa, Haemophilus influenzae
VNIHGLAKLNGNVTLIDHSQFTLSNNATQTGNIKLSNHANATVNNATLNGNVHLTDSAQFSLKNSHFWHQIQGDKDTTVTLENATWTMPSDTTLQNLTLNNSTVTLNSAYSASSNNAPRHRRSLETETTPTSAEHRFNTLTVNGKLSGQGTFQFTSSLFGYKSDKLKLSNDAEGDYTLSVRNTGKEPVTLEQLTLIESLDNKPLSDKLKFTLENDHVDAGALRYKLVKNKGEFRLHNPIKEQELLNDLVRAEQAEQTLEAKQVEQTAEKQKSKAKARSRRAVLSDTPSAQSLLNALEAKQVEQTTETQTSKPKTKKGRSKRALSAAFSDTPFDLSQLKVFEVKLEVINAQPQVKKEPQDQEEQGKQKELISRYSNSALSELSATVNSMFSVQDELDRLFVDQAQSALWTNIAQDKRRYDSDAFRAYQQKTNLRQIGVQKALDNGRIGAVFSHSRSDNTFDEQVKNHATLTMMSGFAQYQWGDLQFGVNVGAGISASKMAEEQSRKIHRKAINYGVNASYQFRLGQLGIQPYLGVNRYFIERENYQSEEVKVQTPSLAFNRYNAGIRVDYTFTPTNNISVKPYFFVNYVDVSNANVQTTVNSTMLQQSFGRYWQKEVGLKAEILHFQLSAFISKSQGSQLGKQQNVGVKLGYRW

SEQ ID NO: 187 - protein, 709 aa, Haemophilus influenzae
MKKTVFRLNFLTACVSLGIASQAWAGHTYFGIDYQYYRDFAENKGKFTVGAKNIEVYNKEGQLVGTSMTKAPMIDFSVVSRNGVAALVGDQYIVSVAHNGGYNDVDFGAEGRNPDQHRFTYQIVKRNNYQAWERKHPYDGDYHMPRLHKFVTEAEPVGMTTNMDGKVYADRENYPERVRIGSGRQYWRTDKDEETNVHSSYYVSGAYRYLTAGNTHTQSGNGNGTVNLSGNVVSPNHYGPLPTGGSKGDSGSPMFIYDAKKKQWLINAVLQTGHPFFGRGNGFQLIREEWFYNEVLAVDTPSVFQRYIPPINGHYSFVSNNDGTGKLTLTRPSKDGSKAKSEVGTVKLFNPSLNQTAKEHVKAAAGYNIYQPRMEYGKNIYLGDQGKGTLTIENNINQGAGGLYFEGNFVVKGKQNNITWQGAGVSIGQDATVEWKVHNPENDRLSKIGIGTLLVNGKGKNLGSLSAGNGKVILDQQADEAGQKQAFKEVGIVSGRATVQLNSTDQVDPNNIYFGFRGGRLDLNGHSLTFKRIQNTDEGAMIVNHNTTQVANITITGNESITAPSNKKNINKLDYSKEIAYNGWFGETDKNKHNGRLNLIYKPTTEDRTLLLSGGTNLKGDITQTKGKLFFSGRPTPHAYNHLDKRWSEMEGIPQGEIVWDYDWINRTFKAENFQIKGGSAVVSRNVSSIEGNWTVSNNANATFGVVPN

SEQ ID NO: 188 - protein, 131 aa, Haemophilus influenzae
VGENAMNLSRRDFMKANAAMAAATAAGLTIPVKNVVAAESEIKWDKAVCRFCGTGCAVLVGTKDGRVVASQGDPDAEVNRGLNCIKGYFLPKIMYGKDRLTQPLLRMTNGKFDKNGDFAPVSWDFAVQNNG

SEQ ID NO: 189 - protein, 721 aa, Haemophilus influenzae
LIRTAILRQFLGILPFKTMAEKFKEAFKKNGQNAVGMFSSGQSTIWEGYAKNKLWKAGFRSNNVDPNARHCMASAAVAFMRTFGMDEPMGCYNDIEQADAFVLWGSNMAEMHPILWSRITDRRISNPDVRVTVLSTYEHRSFELADHGLIFTPQTDLAIMNYIINYLIQNNAINWDFVNKHTKFKRGETNIGYGLRPEHPLEKDTNRKTAGKMHDSSFEELKQLVSEYTVEKVSKMSGLDKVQLETLAKLYADPTKKVVSYWTMGFNQHTRGVWVNQLIYNIHLLTGKISIPGCGPFSLTGQPSACGTAREVGSFPHRLPADLVVTNPKHREIAERIWKLPKGTVSEKVGLHTIAQDRAMNDGEMNVLWQMCNNNMQAGPNINQERLPGWRKEGNFVIVSDPYPTVSALSADLILPTAMWVEKEGAYGNAERRTQFWRQQVKAPGEAKSDLWQLMEFAKYFTTDEMWTEDLLAQMPEYRGKTLYEVLFKNGQVDKFPLSELAEGQLNDESEYFGYYVHKGLFEEYAEFGRGHGHDLAPFDMYHKARGLRWPVVEGKETLWRYREGYDPYVKEGEGVAFYGYPDKKAIILAVPYEPPAESPDNEYDLWLSTGRVLEHWHTGTMTRRVPELHRAFPNNLVWMHPLDAQARGLRHGDKIKISSRRGEMISYLDTRGRNKPPRGLVFTTFFDAGQLANSLTLDATDPISKETDFKKCAVKVEKAA

SEQ ID NO: 190 - protein, 65 aa, Haemophilus influenzae; identical to SEQ ID NO: 176

SEQ ID NO: 191 - protein, 216 aa, Haemophilus influenzae
LVMFNDFLATFSQQLTPQMWGVVATATYETVYISFASTLLAVLVGVPVGIWTFLTGKNEILQNNRTHFVLNTIINIGRSIPFIILLLILLPVTRFIVGTVLGTTAAIIPLSICAMPFVARLTANALMEIPNGLTEAAQAMGATKWQIVRKFYLSEALPTLINGVTLTLVTLVGYSAMAGTQGGGGLGSLAINYGRISQYALCNLGGNHYYCAIRYD

SEQ ID NO: 192 - protein, 65 aa, Haemophilus influenzae; identical to SEQ ID NO: 176

SEQ ID NO: 193 - protein, 45 aa, Haemophilus influenzae
LRKDALPAFFTDVNQMYDALLNKSGATGVFTDFPDTCVEFLKGIK

SEQ ID NO: 194 - protein, 65 aa, Haemophilus influenzae; identical to SEQ ID NO: 176

SEQ ID NO: 195 - protein, 170 aa, Haemophilus influenzae
LPKPEPIPRPRRLALCFAPSAGDRVFKRISYSSTLTMYETWLIIPRTAGVSINSTVWCIWRRPRPRKVALCFGKRAIELRTCVTLIVLAIIHYPKISSTVLPRFAATISGDFIFANASIVARTTLIGLVEPYALERTLRTPATSNTARIAPPAMIPVPSLAGCINTREPV

SEQ ID NO: 196 - protein, 335 aa, Haemophilus influenzae
LFIYGGINMQITLSNTLANDAWGKNAILSFDSNKAMIHLKNNGKTDRTLVQQAARKLRGQGIKEVELVGEKWDLEFCWAFYQGFYTAKQDYAIEFPHLDDEPQDELLARIECGDFVRGIINEPAQSLTPVKLVERAAEFILNQADIYNEKSAVSFKIISGEELEQQGYHGIWTVGKGSANLPAMLQLDFNPTQDSNAPVLACLVGKGITFDSGGYSIKPSDGMSTMRTDMGGAALLTGALGFAIARGLNQRVKLYLCCAENLVSNNAFKLGDIITYKNGVSAEVLNTDAEGRLVLADGLIEADNQNPGFIIDCATLTGAAKSGCRKRLSFCIIYG

SEQ ID NO: 197 - protein, 121 aa, Haemophilus influenzae
VAVGNDYHSVLSMDDELVKNLFQSAQAENEPFWRLPFEDFHRSQINSSFADIANIGSVPVGAGASTATAFLSYFVKNYKQNWLHIDCSATYRKSGSDLWSVGATGIGVQTLANLMLSRSLK

SEQ ID NO: 198 - protein, 841 aa, Haemophilus influenzae
LPIELKVEGLVGKPNEKISAAEFRQKCREYAAEQVEGQKKDFIRLGVLGDWDNPYLTMNFDTEANIIRTLGKVIENGHLYKGSKPVHWCLDCGSSLAEAEVEYEDKVSPSIYVRFPAESADEIEAKFSAQGRGQGKLSAIIWTTTPWTMPSNRAIAVNADLEYNLVQLGDERVILAAELVESVAKAVGIEHIEILGSVKGDDLELSRFHHPFYDFTVPVILGDHVTTDGGTGLVHTAPDHGLDDFIVGKQYDLPMAGLVSNDGKFISTTEFFAGKGVFEANPLVIEKLQEVGNLLKVEKIKHSYPHCWRHKTPIIFRATPQWFIGMETQGLRQQALGEIKQVRWIPDWGQARIEKMVENRPDWCISRQRTWGVPMTLFVHKETEELHPRTLDLLEEVAKRVERAGIQAWWDLDEKELLGADAETYRKVPDTLDVWFDSGSTYSSVVANRLEFNGQDIDMYLEGSDQHRGWFMSSLMLSTATDSKAPYKQVLTHGFTVDGQGRKMSKSIGNIVTPQEVMDKFGGDILRLWVASTDYTGEMTVSDEILKRAADSYRRIRNTARFLLANLNGFDPKRDLVKPEKMISLDRWAVACALDAQNEIKDAYDNYQFHTVVQRLMRFCSVEMGSFYLDIIKDRQYTTKADSLARRSCQTALWHIAEALVRWMAPILSFTADEIWQHLPQTESARAEFVFTEEFYQGLFGLGEDEKLDDAYWQQLIKVRSEVNRVLEISRNNKEIGGGLEAEVTVYANDEYRALLAQLGNELRFVLITSKVDVKSLSEKPADLADSELEGIAVSVTRSNAEKCPRCWHYSDEIGVSPEHPTLCARCVENVVGNGEVRYFA

SEQ ID NO: 199 - protein, 33 aa, Haemophilus influenzae
LENKMTVDYKNTLNLPETSFPMRGDLAKREPDK

SEQ ID NO: 200 - protein, 35 aa, Haemophilus influenzae
MKITHCKLKKSIQNKLLEFFVLEVTARAAADLLDI

SEQ ID NO: 201 - protein, 167 aa, Haemophilus influenzae
LFLVGNLLRWVWLALFIIAQIWAYVQTPDSWLAMISGISGILCVVLVSKGKISNYFFGLIFAYTYFYVAWGSNFLGEMNTVLYVYLPSQFIGYFMWKANMQNSDGGESVIAKALTVKGWMTLIVVTTVGTLLFVQALQAAGGSSTGLDGLTTIITVAAQILMILPLS

SEQ ID NO: 202 - protein, 248 aa, Haemophilus influenzae
MFSGEHDACDCYVDLQAGSGGTEAQDWTEMLLRMYLRWAESKGFKTELMEVSDGDVAGLKSATIKVSGEYAFGWLRTETGIHRLVRKSPFDSNNRRHTSFSAAFVYPEIDDDIDIEINPADLRIDVYRASGAGGQHVNKTESAVRITHMPSGIVVQCQNDRSQHKNKDQAMKQLKAKLYELELQKKNADKQAMEDNKSDIGWGSQIRSYVLDDSRIKDLRTGVENRNTQAVLDGDLDRFIEASLKAGL

SEQ ID NO: 203 - protein, 81 aa, Haemophilus influenzae
LLGNEKQAEAQAKYAEDTLKQARDFAKQHHKTAYLARNADGLQTGQKGSIHTEAMELVGLENVAEGEQKGLTQVSMEQLLL

SEQ ID NO: 204 - protein, 178 aa, Haemophilus influenzae
LPRIFAACFVGAALACGGATYQGMFKNPLVSPDILGVSAGAGFGASLAIFYNLPMIYIQFFAFSGGILAVLCVSLIASRSRTQDPILVLVLSGIAIGSLLGAGISLLKILADPFTQLPSITFWLLGSLTAINQQDLIQLIPMLLLGIVPIFLLLTDTLARTIAPIELPLGILTSACGY

SEQ ID NO: 205 - protein, 65 aa, Haemophilus influenzae
LKNSLRELKDYTVVIVTHNMQQATRCSDYTAFMYLGELVEFGQTQQIFDRPKIQRTEDYIRGKMG

SEQ ID NO: 206 - protein, 206 aa, Haemophilus influenzae
MISLQETKIAVQNLNFYYEDFHALKNINLRIAKNKVTAFIGPSGCGKSTLLRSFNRMFELYPNQKATGEINLDGENLLTTKMDISLIRAKVGMVFQKPTPFPMSIYDNIAFGVRLFEKLSKEKMNERVEWALTKAALWNEVKDKLHKSGDSLSGGQQQRLCIARGIAIKPSVLLLDEPCSALDPISTMKIEELITGVKLYCGYSNS

SEQ ID NO: 207 - protein, 65 aa, Haemophilus influenzae
MSQLNIQFPTKFKPLFESIWRFIIFYGGRGSGKSFSIARALVLRAYQSPVRVLCSVKFRNRFLIL

SEQ ID NO: 208 - protein, 285 aa, Haemophilus influenzae
VVPEFIIVSLILVAQSMKLALNKWLIIFGNAIALHIKYALLRLNFEGVVGEILEKVDNGQMGVVLKRMMVRAASKVAQRFNIEAIVTGEALGQVSSQTLTNLRLIDEAADALVLRPLITHDKEQIIAMAKEIGTDDIAKSMPEFCGVISKNPTIKAVREKILKEEGHFNFEILESAVQNAKYLDIRQIAEETKAVVEVEAISVLGENEVILDIRSPEETDEKPFESGTHDVIQMPFYKLSSQFGSLDQSKSYVLYCERGVMSKLQALYLKENGFSNVRVFAKNIH

SEQ ID NO: 209 - protein, 108 aa, Haemophilus influenzae
LAIAIGGGNRGNASGVLRQNFAEDKAKKTASKLVGVMAHYFGGKSFYLPAGDKIKEALRDAQIYQEFNGKNVPDLIKKYRLSESTIYAILRNQRTLQRKRHQMDFNFS

SEQ ID NO: 210 - protein, 273 aa, Haemophilus influenzae
LFRWHYLGGFTVMPDTNNTETNNKIELYLNGKILSGWKSLNLQRSLESMSGRFDLGIAVRPEDDISVLAAGSPLVLKMGGQTVITGYLDEIKQRVSGNDKTISVSGRDKTCDLVDCAIIHNSYQFKNQTAKQIAEAICKPFGISVVWQVQAPEANERIPVWQVEPGETAFDNLSKIARHKGVLVTSDVDGNLLFTEPSNKQVGNLTLGENLLELEQTDSWLQRFSLYRVIGDAEQGGAKGDTKTKNKAAKGKEKDDGVVEDPDIYPGPAEGGK

SEQ ID NO: 211 - protein, 171 aa, Haemophilus influenzae
MKVSYRLNNCLSLKLALIPLLILLFVVMGSVLSLIAKLDFYFFQQILFNSELHFALLMSLGTSLFSLILALCIAIPSAWRMSQVRLPFQSFFDTLFDLPMVLPPLVTGLSLLLLFSSQGILAELLPFISKWIFSPVGIIIAQTYIASSILLRCSEPLKLRKKTIKTTKIKP

SEQ ID NO: 212 - protein, 670 aa, Haemophilus influenzae
LTKRKNVSFTYENYTVTPFWDTLKLSYSQQRITTRARTEDYCDGNEKCDSYKNPLGLQLKEGKVVDRNGDPVELKLVEDEQGQKRHQVVDKYNNPFSVASGTNNDAFVGKQLSPSEFWLDCSIFNCDKPVRVYKYQYSNQEPESKEVELNRTMEINGKKFATYESNNYRDRYHMILPNSKGYLPLDYKERDLNTKTKQINLDLTKAFTLFEIENELSYGGVYAKTTKEMVNKAGYYGRNPTWWAERTLGKSLLNGLRTCKEDSSYNGLLCPRHEPKTSFLIPVETTTKSLYFADNIKLHNMLSVDLGYRYDDIKYQPEYIPGVTPKIADDMVRELFVPLPPANGKDWQGNPVYTPEQIRKNAEENIAYIAQEKRFKKHSYSLGATFDPLNFLRVQVKYSKGFRTPTSDELYFTFKHPDFTILPNPNMKPEEAKNQEIALTFHHDWGFFSTNVFQTKYRQFIDLAYLGSRNLSNSVGGQAQARDFQVYQNVNVDRAKVKGVEINSRLNIGYFFEKLDGFNVSYKFTYQRGRLDGNRPMNAIQPKTSVIGLGYDHKEQRFGADLYVTHVSAKKAKDTYNMFYKEQGYKDSAVRWRSDDYTLVDFVTYIKPVKNVTLQFGVYNLTDRKYLTWESARSIKPFGTSNLINQGTGAGINRFYSPGRNYKLSAEITF

SEQ ID NO: 213 - protein, 248 aa, Haemophilus influenzae
LRERSSLSALMAKTIEWDFITENPLKYLEKPKAPAPRTRRYNEHEIERLIFVSGYDVEHIEPPKTLQNCTGAAFLFAIETAMRAGEIASLTWNNINFEKRTTFLPITKNGHSRTVPLSVKAIEILQHLTSVKTESDPRVFQMEARQLDHNFRKLKKMEGLENANLHFHDTRRERLAEKVDVMVLAKISGHRDLSILQNTYYAPDMAEGYKTKAGYDLTPTKGLSQRNFFFFNENFIVFTTNPPIVIKL

SEQ ID NO: 214 - protein, 105 aa, Helicobacter pylori
MATIIKNGKRWHAQVRKFGVSKSAIFLTQADAKKWAEMLEKQLESGKYNEIPDITLDELIDKYLKEVTVTKRGKREERIRLLRLSRTPLAAISLQEIGKAHFREW

SEQ ID NO: 215 - protein, 529 aa, Helicobacter pylori
MEAVQLDKNQEPNYKGYSGSLIHPAFQQQTTKREKPSTPLPSLDLLLKYPPNEQRITPDEIMETSQRIEQQLRNFNVKASVKDVLVGPVVTRYELELQPGVKASKVTSIDTDLARALMFRSIRVAEVIPGKPYIGIETPNLHRQMVPLRDVLDSNEFRDSKATLPIALGKDISGKPVIVDLAKMPHLLVAGSTGSGKSVGVNTMILSLLYRVQPEDVKFIMIDPKVVELSVYNDIPHLLTPVVTDMKKAANALRWCVDEMERRYQLLSALRVRNIEGFNEKIDEYEAMGMPVPNPIWRLGDTMDAMPPALKKLSYIVVIVDEFADLMMVAGKQIEELIARLAQKARAIGIHLILATQRPSVDVITGLIKANIPSRIAFTVASKIDSRTILDQGGAEALLGRGDMLYSGQGSSDLIRVHGAYMSDDEVINIADDWRARGKPDYIDGILESADDEESSEKGISSGGELDPLFDEVMDFVINTGTTSVSSIQRKFSVGFNRAARIMDQMEEQGIVSPMQNGKREILSHRPEY

SEQ ID NO: 216 - protein, 298 aa, Helicobacter pylori
MNKIFKVIWNVVTQTWVVVSELTRAHTKRTSATVATAVLATVLSATVQAINDAGTFVKVQSTEDDIEDSAATKDDNKNQALKAGDTLTLKAGKNLKAKLDQGGKSVTFALAKDLDVKTAKVSDTLTIGGNTPAAGGATPKVSITSTADGLKLAKGTNGDTAVHLNGLASTLPDVTTNTGASTSVTFSPSDIEKTRAATIKDVLNAGWNIKGAKVAGGNTENVDLVAGYDNVEFITGDKNTLDVVLTAKENGKTTEVKFTPKTSVIKDNNGKLLTGKQLKDANTGTATNATEDTDEAMA

SEQ ID NO: 217 - protein, 65 aa, Helicobacter pylori; identical to SEQ ID NO: 176

SEQ ID NO: 218 - protein, 112 aa, Helicobacter pylori
MFAVHAAMITTLKKEVFFLYLYIKSLKIPIPTTLKYMISLGKIRELDVLANLAKLCPTCHRALKKGSSEEEFQKRLIRNILNRNKDNLEFAQLRFETDDFSTLIDRICESLK

SEQ ID NO: 219 - protein, 265 aa, Helicobacter pylori
MIKQTLIILAPFFIATLLYFLGAPDGLRPNAWLYFCIFMGMIIGLILEPVPSGLIALSALVLCIALKIGASDKVASANKAISWGLSGYANKTVWLVFVAFILGLGYEKSLLGKRIALLLIRFLGQTPLGLGYAIGLSELCLAPFIPSNSARSGGILYPIVSSIPPLMGSTPNNNPDKIGAYLMWVALASTCITSSMFLTALAPNPLAMEIAAKMGVNEISWFSWFLAFLPCGVVLILLVPLLAYKTCKPTLKGSKEVSLWAKKRN

SEQ ID NO: 220 - protein, 72 aa, Helicobacter pylori
MSRHRGAKPPRRCELLGEISLLSPGYLLSFERWPFHTEPPDHYDRLSSLLDLYVLQSGWLVPLHSTCDFQPQ

SEQ ID NO: 221 - protein, 294 aa, Helicobacter pylori
VQLHCHNLPCVSIDILLGGPPCQSYSTLGKRKMDEKANLFKEYLRLLDLVKPKIFVFENVVGLMSMQKGQLFKQICNAFKERDYILEHAILNALDYGVPQMRERVILVGVLKSFKQKFYFPKPIKTHFSLKDALGDLPPIQSGENGDALGYLKNADNVFLEFVRNSKELSEHSSPKNNEKLIKIMQTLKDGQSKDDLPESLRPKSGYINTYAKMWWEKPAPTITRNFSTPSSSRCIHPRDSRALSIREGARLQSFPDNYKFCGSGSAKRLQIGNAVPPLLSVALAQAVFDFLKG

SEQ ID NO: 222 - protein, 89 aa, Helicobacter pylori
LMEFDVTIIDETGRATAPEILIPALRTKKLILIGDHNQLPPSIDRYLLEQLESDDIQNLDAIDRQLLEESFFENLYKYIPESNKAMLNE

SEQ ID NO: 223 - protein, 184 aa, Helicobacter pylori
MPASIGSLVSQLFYKEKLKNGVIKNTSQFYDPKNIIRWINVEGEHQLEKTSSYNKNQVQKIIELLEQINRVLNQRKIRKTIGIITPYNAQKRCLRSEVEKYGFKNFDELKIDTVDAFQGEKADIIIYSTVKTYGNLSFLIDSKRLNVAISRAKENLIFVGKKSFFENLRSDEKNIFSAILQVCR

SEQ ID NO: 224 - protein, 216 aa, Helicobacter pylori
LIIETQQDPKELPESCKITPQKISFNQVVFKKIKRKLNRFIGSILARTEVYKNLVAKYDELTGKYESLLAKEANIKETFWERRADSEKEAFFLEHFYLTSVYVASTAGYYITPKGAKTFIEATERFKIIEPVDMFINNPTYHDVANFTYLPCPVSLNKHAFNSTIQNAKKPDISLKPPRKSYFDNLFYDQLNTRKCLKAFHKYSRRYAPLKTPKEV

SEQ ID NO: 225 - protein, 293 aa, Helicobacter pylori
LMEILVLNLGSSSIKFKLFDMKENKPLASGLAEKIGEEIGQLKIKSHLHHNDQELKEKFVIKDHASGLLMIRENLTKMGIIKDFNQIDAIGHRVVQGGDKFHAPVLVNEKVMQEIGNLSILAPLHNPANLAGIEFVQKAHPHIPQIAVFDTAFHATMPSYAYMYALPYELYEKYQIRHYGFHRTSHHYVAKEAAKFLNTAYEEFNAISLHLGNGSSAAAIQKGKSVDTSMGLTPLEGLIMGTRCGDIDPTVVEYTAQCANKSLEEVMKMLNHESGLKGICGDNEKHRSQKRKR

SEQ ID NO: 226 - protein, 73 aa, Helicobacter pylori
MPNSQVAGQASVFIFPDLNAGNIAYKAVQRSAKAVAIGPILQGLNKPINDLSRGALVEDIINTVLISALQAQD

SEQ ID NO: 227 - protein, 123 aa, Helicobacter pylori
VSLVSSVFLMCLDTQVLVFGDCAIIPNPSPKELAEIATTSAQTAKQFNIAPKVALLSYATGDSAQGEMIDKINEALTIAQKLDPQLEIDGPLQFDASIDKSVAKKKCLTAKWLGKLAFLFSRI

SEQ ID NO: 228 - protein, 98 aa, Helicobacter pylori
LKAAHRLNLMGAVGLILLGDKEAINSKNLNLNLENVEIIDPNTSHYREEFAKSLYELRKSKGLSEQEAKQLVLDKTYFATMLVHSGYVHAMVSGVNHS

SEQ ID NO: 229 - protein, 285 aa, Helicobacter pylori
VKQISISCSHRKYFVSFSVEYEQDITPIKNTKNGVGLDLNILDIACSCEINNHDKLTDFKQYQTDMKELLGIEIDEELDTKRLIPTYSKLYSLKKYSKKFKRLQRKQSRRVLKSKQNKTKLGGNFYKTQKKLNQAFDKSSHQKTDRYHKITSELSKQFELIVVEDLQVKNMTKRAKLKNVKQKSGLNQSILNASFYQIISFLDYKQQHNGKLLVKVPPQYTSKTCHCCGNINHKLKLNHRQYWCLECGYREHRDINAANNILSKGLSLFGVGNIHADFKEQSLSC

SEQ ID NO: 230 - protein, 157 aa, Mycobacterium tuberculosis
MKVNKGFKFRLYPTKEQQDKLQHCFFVYNQAYNIGLNELQEQYETNKDSPPKERKYKKSSELDNAIKQCLRARDLPFSAVIAQQARMNVERALKDAFKVKNRGFPKFKNSKSAKQSFSWNNQGFSIKESDDECFKTFTLMKMPLLMRMHRDFPLILK

SEQ ID NO: 231 - protein, 107 aa, Mycobacterium tuberculosis
LIFITHFSTEPLPLPILVSKGLAVKGLSGNTLIHTLPALLMCLVMATLADSIWRESILPCSMALIAISPNAMELPRWAFPRLRPFICFLYFVLFGINMIIASLFCFF

SEQ ID NO: 232 - protein, 72 aa, Mycobacterium tuberculosis; identical to SEQ ID NO: 220

SEQ ID NO: 233 - protein, 195 aa, Mycobacterium tuberculosis
LNAAFKERRFILVQLDEKIDPKEDKSAYDFCLNTLKSPSPSIFDITEERIKRAGAKIKEACAHLDVGFRAFEIIDDETHANDKNLSQAHQKDLFAYSNLDRMETQTILIKLLGCEGLELTTPITCLIENALYLALNTAFIVGDIEMSEVLENLKDKGVEKISMYMPAISNDNLCLELGSNLLDLKLESGDLKIRG

SEQ ID NO: 234 - protein, 234 aa, Mycobacterium tuberculosis
MYIRFYRDSLAEPATDIYAFAYVSFNKEAGTWHTPAQPTRNYGSGTPMTTAATAPLRHAPMSGRPPKRGSNACAGARSYSSAGVLNTRSSIGWSTAYGPASSFPAASTESANSSRQPTTCCVGLPAARSIPGSSRTMRLCWPATKDSRSPRCPGSWCTCRSHRLAHNRPLDARSASPAVAKPSVIRLGSRVRRRSGSPDHLPSARICVSSRRSPRRLLWCYRRPLARCSESTIR

SEQ ID NO: 235 - protein, 169 aa, Mycobacterium tuberculosis
LMFCASRKEMAMSNSSSSSVINWNSLSESKPRSSTSTWFAVMPRSVRKIRWMVALMASFIARLLAGSGPRQGRQTRARPGRGQIVGGRLGSWCGIPNAPPARLGGPPGSHTPRSASAADSPHAPRSGCPGSPARSRFRDTRPDSPAVPGRWPCTRPRPAPEPAGRVHAD

SEQ ID NO: 236 - protein, 187 aa, Mycobacterium tuberculosis
VPPPIPRCAAASTSDPMASVKYGATRRWWPPAPSLTTSSCSAACGLCPKSSPGSSIPSDEPDSTATVGQHSTMLTATLMASPPAEVSLYLVCMSAPVARMVSMTWSRVTRWMPSPRNAIRAALMALPAEIALRSMQGICTSPAMGSQVRPRLCSMAISAAFSTCRGVPPRISANPAAAMAAAEPTSP

SEQ ID NO: 237 - protein, 263 aa, Mycobacterium tuberculosis
MIPMDVIFGCPLYANFCKPSVVRKTLGILASAVEFGTMPTTRNLRAPTSTVSPRCRPIVLDAATSSGFDGDRPSETRGMPGPCSGAPKTVTFRVDVPSFMIVPTLPNGAAAMTPGSAATRARSTSGNGIEPRKGPAAPDLTTNTSTPMESTVCRASTRKPFASPVKTSVIPKISPVLMIVMTRRRFLHCMSRRAAKSIPRGYQRGALVGPGLDVLWSGRGPLVVEEAFGVVVVVGVGTAVEVGWRDPFRLAVGPFPCLPAFPD

SEQ ID NO: 238 - protein, 281 aa, Mycobacterium tuberculosis
MSRAIRTKPKSASSRGSAGYRVYLGQRLGVAVFAQRDDHPVTGLEDADERAVISGPVGAHTVAMPLDHYRFTLVDAADEFDVDLEDLLAPLDCSPKRLLVQFRTGDDAPVGEVVAEQREAFVEISALAEALQEHPGQFGLRVVERRHHIAILSRETACGQLTWSSKRWSPSRGRPASRTPWRRCVAVSRIHAFGSPVTALSGGPACCPPGRSPRGSAVLGATPPVAWRGAAVPRSLSTWRPPCWAPPTTPAISCRCIRPWPPRTAGCRTCAWAAPARCWKP

SEQ ID NO: 239 - protein, 163 aa, Mycobacterium tuberculosis
VRPGHRQVDGCRRGQPLCGAHERVGLVRVVGAFGLAQQGCDAGQHLVVGHGAKTSGGLRQVGSAYNRSVSQATTSSSTWLRSGSLNTSWYSPGYSFSCTSADPTHSTRRRLPSMGISRSSVPCSTSSGAVNAGARRGMVSPTCSSARPIPAGTRPWCTSGSVL

SEQ ID NO: 240 - protein, 171 aa, Mycobacterium tuberculosis
VCKACLGHHTHHHRTSRPLRNRCQHDQPRPAHRHGFHPNPRFRRQRHRGRVPLRLRLAAEPGILQLDHNPVVGLLQLRRRWRIGLPQRRRSRRVGPGKRLHRDFGLLQCWRRRNSGFQNFGNLLSGWANLGNTVSGFYNTSMLDLATQALISGFGNHGARLSGILNNGSGP

SEQ ID NO: 241 - protein, 586 aa, Mycobacterium tuberculosis
VLSLSAGGPEPRMRPGHNPVTFHAEQTRNRTARTSRVRFRVCSSDKSAQDQRVGVGADVDRHGIAVVHLAGQQHLGQLVTDGLLHQPAQRPRPVHRVESALRQPALGRQRDLQLQPPLRQPLAQLRQLDVDDAHQLFGVETLKDEHVVEPVDELRLERSAHRGQHLLGAATRPQVGRQDQDGVAEVDRAAVPVGEPALVEDLQQHVEHVRVRLLDLVEQHHRVGTPAHRLGQLTARLVSHIAGRGADQPSHGVLLAVLAHVDADHRPLVVEQEVGQRLGQLGLADTGRAEEHERPGGPVGVGHPGPAAPHRIRDCGNRGLLPDDPLAQLVFHAQQLGGLAFQQPTGRDAGPRRHHVGDVVGTDLLLEHHLLPGLRLRQRRVELLLHLGDASVAQLGGLGQVAVAFGPLGFPAQGFQLLLEVADDFDRVLLVLPAGGELGQLLFLVGQLGAQLGQPLRRRLVFFFGQRHLFDLQPAHQPLDLVDLDGPRVDLHPQPAGRLVDQVDGLVGQEAGGDIPVAQSGSCHQRRVGDAHPVVHLVAVFEPAQDADGVLHRRLADVHLLETALERGVLLDVLAVFVQRGRPDQP

SEQ ID NO: 242 - protein, 371 aa, Mycobacterium tuberculosis
LLADFDVGQHLFQLVVGGLGTQHGFGVQRVALPDRLGPDRRQLQELVVDVGLDQTARRAGAHLALVEGEHGEAFQRLVAEVVVGGQHVGEEDVGALAAEFQGDRDQVVRGVLHDQPPRGGFPGERDLGDAVAGGQRLAGLGAESVDHVDHPGRQQITDQRHQVEHRSGCLLGGFEHRRVAGRQRRRQLPGRHQDGEVPRNDLAHHAERLVEVVGHGVLVDLAQRALLGANRRGEVPEVIDRQRDIGGQRFPDRFPVVPDLGHRQRGGVLVDAVGNHVEDRRPFGRCGLAPPRRRRVRGVERLVDVGRVGARHLAERLAGHRRRVLEVAPMDRRDPLAPDEVLVPGFIGHQRPGGTGTGKDSHRIRLLVKIM

SEQ ID NO: 243 - protein, 153 aa, Mycobacterium tuberculosis
VYLPPKLIPRRIPAQVRPTMVAPQVPHVLSITPNGRSGEVCPASGSTRPKLGVQPPAASGWPLPTRPGPRFSRCHRRPTLPACARSSSATGSTPKSDNPANPAGTSSRGGRSSTTRRCWLPAAIRAALKSRFSARPTDSGAVGRAGRPHPAQA

SEQ ID NO: 244 - protein, 78 aa, Mycobacterium tuberculosis
MTSTNGPSARDTGFVEGQQAKTQLLTVAEVAALMRVSKMTVYRLVHNGELPAVRVGRSFRVHAKAVHDMLETSYFDAG

SEQ ID NO: 245 - protein, 132 aa, Mycobacterium tuberculosis
VAESVAIRGCLLRCGPRSRPRRRSRRSGICACRPRCSATSRPPCPRRSTCPPRRRSMTSAPSMWPPGRQRSRASRCIATAAGKDRYCPTPRRNRYWRRLTRSSAAAVRAAPASSDGGSHGASRRRIAQNQRF

SEQ ID NO: 246 - protein, 84 aa, Mycobacterium tuberculosis
LLHSSFGHLEGIQQPLIDELAELDHVLGKLPDAYRIIGRAGGIYGDFFNFYLCDISLKVNGLQPGGPVRTVKLFGQPTGRCTPQ

SEQ ID NO: 247 - protein, 293 aa, Mycobacterium tuberculosis
LLGALHQYPHTRIQPGAVAAHRDRQHPRPVFGDEALDAAGVLMRTHAADHRQSEVSTVGLNAHRTRGERHAIGVAALLLESREAHSLAVALASTPLLPVPVRVDRARDPVGVGLFRAFRPPHGASLGVDTHLVFHRVPAFPQYPKRRLRRLGAGRAPRLDIGFQLRDGPVVGLAAGAEMPRQRVCLLGGRIECEPERLHTPAVGDLQTRHLRPPHDHRQRQPRRPAWPGSEQHVCHTTLRTSRSESRSYPIPGHRQPRPSPPRPTPDPERPAQRGHTPNRTGRTDPDAQPQSA

SEQ ID NO: 248 - protein, 55 aa, Mycobacterium tuberculosis
MASSTDVRPKITLACEVCKHRNYITKKNRRNDPDRLELKKFCPNCGKHQAHRETR

SEQ ID NO: 249 - protein, 213 aa, Mycobacterium tuberculosis
LVCAAAPGRRRPLGVGGQVEAGTESLAATGHQNDMHAWIQIGTLHQSRQLQRGVCDDRVALLRPVEGDPRNPTGDLIGHRLQVVEIDRPDRVCHQRPLSLLPAHARGWARDPDRPAWCRTLRPTGRRAEWPETPRRRRDVRGAPTTIPATPGRCLRQSCGLDNRSCQDRPAADAAFRRGRPAWGPGLRCGPARQTAPRRMRAGLPWRARYLAR

SEQ ID NO: 250 - protein, 131 aa, Mycobacterium tuberculosis
LGLVAPAGDGRAAKKRPAGRRGSDRRRRMRLRGVVRPTPARRCHDLWGLHHRVHCHAVAAHRLQNGTGRWSTGASTSMRSTTVASAAARGSRPSTSAETTDPSTAQINVHTSSICAERPERSMASATASAR

SEQ ID NO: 251 - protein, 298 aa, Mycobacterium tuberculosis
MRCRAALSWRLPERLSRIWPAVLPDHTGMGATAAWQAKAASLLNRVTPAASPTILAAVSSAQPGISSSAGATWWTRALMRWARVLISPVSRMMSVSSARASSATNPGWVSSQVRRACWCLAASSERAAGARSGSSSWTSQRNRLIADVRWATRTSRRSVNNFNSRDVSSWVARGRSVSRITARATASASIGSDLPRLRADLRVWAISLVGTRTTCWPAASRSRSRRADMLRQSSMPQISSRPNCSRAHMMAVACPAVVALTVFSPSWRPTSSVATKVWLYLCASVPTTTMVVASEPPR

SEQ ID NO: 252 - protein, 265 aa, Mycobacterium tuberculosis
LRRRAAVPVGLHRRRSDRAGATQRDRRRYRRWVHACRLCAAWRRDRRTSGPDRARSLRYLCHRRRRRRGGQCAGSRPGQTRRRHHRDGLVGSAFQWVLAGPQGVAGDRPDESGRSCGGVRSHLGRRVIGADSHLRQRLFGLGRRNPCPDVLPRHRRRARRQPATGHPAWPHRRGRPRHLDTRAGIHHDCPARPGQAHRDGEDVQHGCRHDRRRCPRRHDARPGRPDRAAPGLLGIGNRLQRRKTRPAGKTGWAAPEILRTRPNRV

SEQ ID NO: 253 - protein, 248 aa, Mycobacterium tuberculosis
VVAVRIEVVGHRVHHLAGHLEFRGFDLHLLVQHREVGVADLIGPQQRVHHHHLSLAEILDAQRRQPGLVAQREMHDRHPVGLGECLSQQHIRFRRLRIRLQKVAAVEHHRVHVGGGDELQHLDLPAAFFRQAGDVVVGDRHHLAVAGLVGPGKIAVVDHLATRLADALVPDASVVLGVHLVEPDVVVCGSAVHLDRHVHQPEGDRTRPNGSHVSEYALIVRERNVTAKFHAIFDRDVTLATCVTDRLR

SEQ ID NO: 254 - protein, 208 aa, Mycobacterium tuberculosis
LRSARVNPPARSAASTPWYPSGSVTTAALGWFLAAARTIAGPPISICSTQSSTLAPDSTVWLNGYKLTTTSSKASIPSCSRAAACSDLRRSASSPACTRGCSVLTRPSSTSGKPVSCSTGVTGIPVSAMVLAVDPVEMISTPAALRPCARSTSPVLSYTLISARRIGRLPSSVLILWLPFVPSSLFVRPPSRHGWPVRPPPLPTAVVR

SEQ ID NO: 255 - protein, 220 aa, Mycobacterium tuberculosis
VRADPPTTACNTRCTPSVCVPSMCGTSTTSMPPRSCVPEKVTLLQSFPGLGAGSGWDVSTAMTTNRLPLPSAETAAMLPCNPVGSWGPAATCAQFAGSKLSPSGSLRAEKNPGSMALGVTSVTVYSGPKPDFTSATLAMSPVEAVVELAPDEQPTSQHTDPTASTALRIVVNLPNAAPELRNVDTVLTSRSAANCGASGGRTDPGSVISRRPRSLAGLPG

SEQ ID NO: 256 - protein, 238 aa, Mycobacterium tuberculosis
VGTAQERVRSRSGPVPHHALRHLRGSPHRGTADPAGDAGVGRQNFGPARPGPKPAVVRRRRCSADPRHSAAAAHRGISPLPAAATTRRQVSGPQRRESHLRSVDRGLRVAWDVERGDGIKPGIVAAVAGQQHGRIVHHMGAVRFVLLPVDRGPQRVVARGQAGQINANRLGDRRRCRLVAAAIAALVGDQRLQVHRCRQRPNHLSGGIHQPVAGHPLFGGGSSAVVGPGDRDRRDLAR

SEQ ID NO: 257 - protein, 238 aa, Mycobacterium tuberculosis
MSISGIERWSATENIRISVISSPQNSTRTGCSAVGAKMSRIPPRTANSPRRPTISTRVYASSTSRATTPSKGDSSPTVSVRGSIMPSCGVMGCSSERTEVTTTPSGGPSWASSGWASRRSAIRRVPTVSTPGESRSCGSVSQDGNNATASPNTPRSSAARSSASRPVAVTTSNGPCRASALATNSRALAGAMSVNSSGRPPARCMSCWNVGALSANSTSPAIGVSEQAGPGAVMMRPF

SEQ ID NO: 258 - protein, 154 aa, Mycobacterium tuberculosis
VLAFYLRPRPGTWCTSEGSSRDPSGGSLGGQCWGVGGLLLGGFFGAGQCCSGSGEDLEAQVAPSFDPFVVLFGEDGSDEADDRGAVGEDAHDVGSASYLSVEAFLGVVGPDLAPDLLGEGGERQQVGAGGVEVLGHRGEFVGQSVEYPIILGNN

SEQ ID NO: 259 - protein, 88 aa, Mycobacterium tuberculosis
LTTAGISGSKGRTGTGEPCGLLSAAGFRAGASGGLTAAERSTARASSANLTRRYLTHAELLMLARATGRFETLTLVLGYCGLRRFTVR

SEQ ID NO: 260 - protein, 181 aa, Mycobacterium tuberculosis
MGQCPRPVRHWPPAVIVCSRTKLRRACLRDYRRPAPSDKKPNKSYRVMTPTGLPSSTTINASQSRNALPAALTNSPAPIIRSGGLMCADTASANLARPSNTAESSSRSETLPATSPAITGGSAPTTGICDTPYSRRIPMASRTVSDGWVCTRAGSAPDLRRNTSPTVDCSVDPSRRLRRNP

SEQ ID NO: 261 - protein, 205 aa, Mycobacterium tuberculosis
LAAIPRRSRCSVNPRGNRHDPARHPGGRGSVRGGDRPELTGDIGLRPGEGSARRGLRPRQAGNRPVRCAQVHEVPTAAILSASSEVFNEVPVRNPGTLAFVPIVDGDLLPDYPVKLAQEGRSHPVPLIIGTNKHESALFRLMRSPLMPITPRDHVDVHPDCRRTARSASANRGADRLRVLAMAAQSTLIEYGYRRRLPDAVGVAR

SEQ ID NO: 262 - protein, 145 aa, Mycobacterium tuberculosis
VLALRPQRHFTQSRSARRLRCVLDDDVWVPWARSGGCRTATRHLSVRCIAGTCWGPPVRFCRLRATPSTVSCSARRRYRSRLTCHRSTDTSWSLSATRLAELLAPLEPVTVTFTPTFGEPDMVHLSGTKFGGLVPALFEGVRAGF

SEQ ID NO: 263 - protein, 286 aa, Mycobacterium tuberculosis
MTSSAPKPAASRASDWPTTSPAPSCSPTANSTVPQSSYAMTSTCWAAGSEWASKPSATGSPHCSARGSEGYRSSSSAPTRPETSQSDSPRRRFTSAGSAAAARCGWSTTRSPSQRGSSARWRKCPTAGRTSGWPRPPLPTGSGIWARTRTSRSGWAATSRTPINSSTPPVSSWTTRARRSRSGRAARSATERRAPNVRSPISVVASRSTRTRAAACLIRRPSNRFDRPTPQQTTKPLILLWFQQALGKHCCRCLHIAFSHVFHSGGDHGGLRVIGYRAVPRAGADL

SEQ ID NO: 264 - protein, 80 aa, Mycobacterium tuberculosis
MQLGNQNTMRFAGRPQRFRQSAYPLFNPNSAIALGHPFGGSGARLMTTVLHHMPDKGIRYGLQTMCEGRGQANATIVELL

SEQ ID NO: 265 - protein, 101 aa, Mycobacterium tuberculosis
VTVYRRGMAVLTDEQVDAALHDLNGWQRAGGVLRRSIKFPTFMAGIDAVRRVAERAEEVNHHPDIDIRWRTVTFALVTHAVGGITENDIAMAHDIDAMFGA

SEQ ID NO: 266 - protein, 103 aa, Mycobacterium tuberculosis
VGAVRLQPHRMGGGMAALHRHTGTADQLLLLPRRAHRAGSPVQCDRLRGRDSHFQPGTNQYRNGHRGIDQPIHQHRDQLDTRLPAAVAANQPAGIPVFALTSD

SEQ ID NO: 267 - protein, 235 aa, Mycobacterium tuberculosis
MPSPVSSGPTSHGTNKGCGLIRSESMNTTMSPLVAASERHNASPLPGRTGTSGNACSRLTTRAPEAMARISVSSVEPESSTINSSTRPSTSGEMLSITDSMVASSLRAGSTTEIVRPAFAASNSPIVQPGRCQVVSKGSAPGALPPARSPATSSDAVMRVLSPCASAAGPPESMPPFPAPAGWRRPHAPETCAPRRPQPTRWLPAFPQAVRSNPRPESPRQRPCCSKPSARATRS

SEQ ID NO: 268 - protein, 122 aa, Mycobacterium tuberculosis
MLSAVILTERGYPAVPLAGQLVHQRFVRPGPLVLGTGFLKFLTRAADRDRTVSRRSKPSSRAALMGEQPNPWDLLQPQDATSRHRGAKPSRRYGLLGKISLLSPGYLLSVERHPFHSGVPDH

SEQ ID NO: 269 - protein, 362 aa, Mycobacterium tuberculosis
LVGRSRVLVLFGAGEHVDVVALLGERAHRLIGEHVVQTVVGHVVQNRNVAVLVTRPAIHQQVGRLRHRLLTAGHHHVELSGPNELISQRDCVDAGQAHLVDRQRRDIPTDAGRHCRLPCGHLPGTRGQHLAHDHVLDQGRRHVGLLQGALNGDGTQLAGAEILQGAHQLADGCTRASNNHRCRYDYLLSAPESRSDRPGEADSFPSGYRCVMTTDQVHARHMLATSLVTGLDHVGIAVADLDVAIEWYHDHLGMILVHEEINDDQGIREALLAVPGSAAQIQLMAPLDESSVIAKFLDKRGPGIQQLACRVSDLDAMCRRLRSQGVRLVYETARRGTANSRINFIHPKDAGGVLIELVEPAP

SEQ ID NO: 270 - protein, 472 aa, Mycobacterium tuberculosis
LRAATKSPSSSCWRACATAGSTSVDSSELAAPLSFPAVADNRESTQRLSWSAGWRPWKLEIGCPAAKATTVGTAWTPNICATLGATSTLTDASDHLPLAAAAKPDSVSSKSTHTSLRGDHSNTTTGTSSDRTITSSSKFASVISVTPDGVDSARSASVLAAAFCWARCLMPERSTAPAMAGPSGGRGRVTPSSLSCRCGHRSTRWRRPCGRSRHTAIGWYDQDHTGRHRPLNRYPARNISASPCPPAPHNAATPTPDPRRANCSAACSVIRVPDMPRGCPTAIAPPLTLTIWGFSPSSRIEANATAANASLISTTSSWSTEMPSRSSALLIALAGCDCSVESGPATTPWAPISASQVSPSSWAFSWFMTTTAAAPSEICDADPAVMVPSPRNAGFRPASAAAVVLARIPSSSVNCSGSPVRCGMFTGITSSANTPSFHAAAAFWWDAAAYSSCSERVNMSTSLRCSVSAPIG

SEQ ID NO: 271 - protein, 244 aa, Mycobacterium tuberculosis
VRSRRLAPTRPRSRRTASPATATRAAAPPRTTPPSAAPATRCPPLARQRNKTRAAQSRLAWRGGRSEQGLSRCGSSGAVLRCGDRHPAALAGVPQPAVASARGKQLLVGAAFDDPTMIEHDDLVGPGDGMQSMGDYQHGAVPGQPVKRLLHKVFRFRIGKRGGLVEDEDRSVAEDGTGNGEPLSLPARKTTVGSEHGIVAVRQPKHPVVDLRFAGRDLDLFGGGIRYRQRDVFGGGAMHKLGFL

SEQ ID NO: 272 - protein, 244 aa, Mycobacterium tuberculosis
VSAVLALSAAVSARRAKAAEAHSAPSSNGTPASAATPSCQEIGNRASAITAGSRIALVNGVTRLTTRPTSSGPVAAIACRAVAVFSAVNQSNRTTGSRSATSCWVWLRTAKPSSIPMRAVTASSTHPATVAADSQPSHSHARCGASPNNAAISGTSNTVPTARATTEQNASSAKPISLARWSFGTRAIQVRIIGCRPALRRPPPGCPGRCPTAGSSVRPRQATPRGCRVRRSDHDRARRSGRPG

SEQ ID NO: 273 - protein, 107 aa, Mycobacterium tuberculosis
MPSVIRDPDPGAAPAPTVADRSAEVPSVLQRSRRCDAYHRYSRWRLSYSASPLGGSRRQPGIATDGRTRGTQPRPAGAAHSRARPDVGRSVAATRPPSAGSAGTARP

SEQ ID NO: 274 - protein, 318 aa, Mycobacterium tuberculosis
VLRPIRAGQPGRHLAPPRPATRRQGTGAGTGARRRLGTGVAPPAGVSVDEPSGCARLGMRVAELPGVAAPHLARPHCRREARAGVGQGKHRRLRRGSEFRCHQRRFGRRPSVRPGGVDPQRSAISARVRTGRHLGGGSGSGIRALRLVYDRCAGASGIRRVARNVRGETEIQHAPRHLRRCLTDPPCAGRRPTVLRSARPPRLPDPRGRSPCVRRGTAGGVEVARRLRGPAPRPTRLRRLRLPAGASHRRGRGPLPVLGVRDQPAGHVVSYRPAIAIPRHAPARPVPVRWHRPSRRCRWPPRVWSPGRNPDNPGRRSR

SEQ ID NO: 275 - protein, 295 aa, Mycobacterium tuberculosis
MTASRRSDHTDATRRALVDAGRYLFARRDYGDVSIEDIVTRARVTRGALDYHFDSKKDLFQTVLEVVEADLVADVEAAIAKVTDAWICWSSASTPSLTRRPNRMRCRSLRLTARQCSGGANGAGSTCARAWSAGRGSRTRDGRRGDSARTVATTFASAAGRANRIRAADRGRDGQRPDQSRGRTRIYGPTRRSTGVARPRSATATDHRPQSRPASRNAPRPATPRRPGHHRRHPGPRCRRRFWRSPSRRRAPAPYRQSSARPTRPTLFGSPHTPPGRRRRWPPARCRSPRPVRRR

SEQ ID NO: 276 - protein, 255 aa, Mycobacterium tuberculosis
VRLRSESAGLAHAADDVSGVVLGDDPDHDPPVAVLDFLVPEDVFPVVVATGQMVVAVILGRDLDVLPAHIQMGFRPAPFVAHRDLRLGARKAGADQQQAQPGFLGGLGTAVDEVQSGSCGLHATAAPIALDQRLDVGHLQIGGLYQGVDGRDGGVQWKSTGQVERRSLRCGHAHALDDADLVGLDALFPDLQPRGTAAVGVDDRGGKIRVDPLGAMEGRSRVAGQHAAAARAQPQRFCTQLRGQFHTLRHVHVFM

SEQ ID NO: 277 - protein, 430 aa, Mycobacterium tuberculosis
LGVRAAVGVDDVTRGRRQPVRQQRAHRLGDRRRILHVPADRSALIPALLEQLDLGAGLLAERTADDRTHRQRPDRACGNEIRTHTVLAGLARHEPVDRLQRALGDRHPVVGRHRPARVEVHADDGTSGVHDRQQRLGHRSIRIRRDVDALGHIRVGRVEERVDAHPGLRHEPNRMHHPVELVARPDRLGHPAGQAGQVLLVLHVEFEQRGLCRQPVGDALNQPQPVEPGEHQLGALLLGYPCDVKRDRRVGDDSANQNPFAVQQSCHVRPCVVSVAHTHAAVDRDDRTGDIARILGSQEADHPGDLGGGADPLRWDKLQRPLLNPLIQRAGHIGVDVARGHHIRGHVCLRQLAGDRAGHANHSGLGGCVVGLVADAPAAGDRTYEYHSTEFVALHAARCPLSHPERPGEVGVDDLLELFLGHPHEECVRG

SEQ ID NO: 278 - protein, 225 aa, Mycobacterium tuberculosis
MVPSMRVRSDWEPIAQSRSRLAVTAPRNTSGGRFIWILLGSARNGSRAPWLPTRSPGSLDRIFLVATDNRTSLPKGRWAPTSRMNPQPRPDVMPWRRATGRSGNPVKRALITGITGPDGSYLAKLPLKGYVAAGSPAEVYFCWATRNYRELYGLLAVNSIWFNHESPRHGETFMTRNPAPYRGRQRGADRCADADAPAHPDRYQYWGVPASVRGVIDRAMGVCVE

SEQ ID NO: 279 - protein, 265 aa, Mycobacterium tuberculosis
LSGQPSALRRPTVSPSACRRPTVSKSKPKIDRMTSRMAPPTTDGSATLNTGHQPTDKKSTTCPRSGPGARKKRSTRLPMAPPRIIPRPSAHHGDTSRRPIQKIPTTTPVAISVKTQVYPVAIEKAAPELRTRVQVTVSPIIDTGWPGGNSWTATTLVTMSSVSTTTATDSSMRSRRGGAGALGSPAPPASSVEVSGSADPVGSSGTPSSSPRADMARPDPAAGWEQTTCAMIPSWPASPSSLLEGQSRPPPAPMGCYGQPIAGRR

SEQ ID NO: 280 - protein, 259 aa, Mycobacterium tuberculosis
VPPNRPERRLGKPLTAPLRRAPAHPLRDGPGSVGNPPKTSRRHQLLRSPKPRRRPGCPQSRTPRTSREAPPATQRPGPPGSGFERRERPAASICGRARRWSAEKRQERTFPGTRRRSRGRWSPRCRARWCRARFGLRQTAARPCGKGCYSKFGHHLADARNARNRLWRMAAAIRVPAAPRRLPGVSGCRDAKLDCITHKRSSPVRGKRVEPVAVVARRHRQSLLGGRGQAKLGGQAQQMHARRLRNRHRRVPVHDTGFR

SEQ ID NO: 281 - protein, 151 aa, Mycobacterium tuberculosis
LWKWKPRLAFHRATWRRRWTATTPTPRAAQIPISTSSRNSLQHKTTGRGGRSTCRWARRCMPDSLWAGWPRRWTVKYCATTARWWPACTRSGHARPISPRTARDMPAGPSWVRGRFSGVAPERMRQPERRACKPPRAATGNPATRHADKAS

SEQ ID NO: 282 - protein, 310 aa, Mycobacterium tuberculosis
LWPRISAAPSNRRSTVGGVWCRRRPNWVSRPRDSRRPCRITTRCAPRGCPLHSPRPSATSSAHTPTAGSTNQASSTHYGVQTAPKYRCSGLELKGGKGVSDEISRRAPTRVRPDIQRRVHRSEPIRGRVALRRRFVHRRRLGHHHSGSGRQYDRGSRAADGRDGRPPRWHRNPAAGSADPGGKADGGVRQKPGPGARHPSDAGTRRFGVRRHGAHPQARTWRRGGHPRGSPDRIGARIVLPGRGSLHPGARYRRDGLCDRSSGNRATQDLRPAGARPGRRCGADRRRRHVGGSAKPHRGYPRRYLHPGHR

SEQ ID NO: 283 - protein, 371 aa, Mycobacterium tuberculosis
LGISPGDRGDRVRGNAAGRDRHPGRLAAFLGADHYSVFSNGPAVEREDRRGQHGAVRTAGRPDGAGAGPQPRTTGAVVTTADPVTAGAAAGSRGYRVALRVRPARPDRALPGGGRHQHRIQYRGCGAGADLCLAAVSGDFPRGCSPHRRSRLRGGGGDTWGAARHCLVARDPAVAAPGRGVRISTGVCPLARRVWRDPNLCRFPARGHPYPSAGDLPAAGDRSGRGGGIVTAARCGSGTGGAGCGCSYADRDRYQVAGHEQAAAARGRRRPAFGRRILGVRGRGACSARAQRCGQVHRPACYRGAASPRRGLGTFGGPGVDRHRGRGECGDPRPSSRAAVARPVVVSTPERGQKRGLRTTMPSRDVWVRAR

SEQ ID NO: 284 - protein, 171 aa, Mycobacterium tuberculosis
LPTPVPARTGTPSRSANPGATGRPTPETANTADCSSSRPPGPHSAVSATQQLPLGNNKSQLPIGFSPNRDWTRGRRAAPPLAFRSHCGRNPRRASSKSSTRSFGQAFRQVFRADGWRRVRSMTRSTYVFGSGHGRFGHSSHGSAAGQDLDIDRGCPQYRPVLAGNLRGRVA

SEQ ID NO: 285 - protein, 202 aa, Mycobacterium tuberculosis
MRRLRSSDPRCHRLHVGARPAPVLPPGQDHRGAFREQRSKSCAARRTRGACESLGAQRGQRRFVVGFLRDFRHQFRVGDVAVRADHHDCAGEQPGHRPVGDGHAVILAEAVPECRRGHDVFGALGAAEALLGERQILRDTQHGSATCRRTLVEGSHTRRAHRCVHGWKDVQQHGLTPELVAADHPQIAPGQGEGRGRGSDSR

SEQ ID NO: 286 - protein, 305 aa, Mycobacterium tuberculosis
LRGSGRTQIQDHAAALSRHPRQRAVEFLAAAARRRAEHVARQALDVDVQRHGHPGTDRTHDDRQMLAEVVNVTKADDTRGAGPGGQRRCRKPDHLGLDPPAIRHQLPDRDHGQSVFDGEFDRLGVVRHLDGIIGRDDLAERGGRPPFRQAGQVDGRLGGSPPTQHTVGLRLHGHHMARTLEIGGDGGGRSQCRDGPGAIARRDSGAGAANVDRHAMRGVSVTHGRQVQSLAFGARQRDAQITRGVPDRKGNQPRRRGLGGEDEIAIAIGVAGQDHGVTARHRRDRTTYPHIGRLHRDSNRRNRLP

SEQ ID NO: 287 - protein, 82 aa, Mycobacterium tuberculosis
MKTAISLPDETFDRVSRRASELGMSRSEFFTKAAQRYLHELDAQLLTGQIDRALESIHGTDEAEALAVANAYRVLETMDDEW

SEQ ID NO: 288 - protein, 77 aa, Mycobacterium tuberculosis
MSTSTTIRVSTQTRDRLAAQARERGISMSALLTELAAQAERQAIFRAEREASHAETTTQAVRDEDREWEGTVGDGLG

SEQ ID NO: 289 - protein, 419 aa, Mycobacterium tuberculosis
VATSTSPAGGLPQARSQPTKCRCPADSTFSDRAASARTSAAECAQPGLPVQALMFSQGEFSSNTRPSGASTRSAASAVASSRSQISTDRHGVITSGASIAARHSATRAGKTPSGTAAPSVTRLSSWGIQPTGVLVTGRTDGPSSTPDCSSPISANSVTRQAVSRILTKRNATSIRVSATSATRTPVSRPVNSSRGPSGNTCTPTSAPRPDTSARPSSRPNQNRPPSSASRGSARIAASSSPTHARTSASPPARPDSGEATILRTRSCVADGSSPALATASATAATSRIPRNWTLPRAVSSSVAEPKSLATLASVASCAAVIIPPGSRIRASAPSAAWCGRNAPGQASASRVPATRPPYGRMGRRLAALRSRREAEDQGQGVFDCAHRGGFEGAESLHESGTSDRADAAAHRDAIGSYTF

SEQ ID NO: 290 - protein, 338 aa, Mycobacterium tuberculosis
MTGRVRQTGITRLVVHQRGPVLPQRLMTVHAGPVVAEQRLGHERDRFAVLPGGVLDDVLVQLHVVGGVQQRIELVVDLGLSAAAHLVVALLQDEAGVDQVGQHLVAQVDVLVVGGHWEIPALVADLVAPVGTAVGLGRRAGVPPPRDGVHLVEGAVGARVEAHRIENVELGLGAEVCGVGDASADQVVLGLAGDVARVAGVRLQGERVVHKEVDIQRLGRAERVDARRLGIGKKQHVGFVDRLEPANRRAVKGQAVVKHALVKGRSRNREVLHDARQVTEPDVDIFDLLVLGKFEDVVGRLFRHRMLLYCIRGRRYGADIARQSTPCCADVTDRAAHH

SEQ ID NO: 291 - protein, 155 aa, Mycobacterium tuberculosis
VAGVCALFSGASRWPSGELRHRPQGSRRGPSRLRCTFPRQNVSSRRPGVPTVGADLTRRSGGTGQPRGMGSPGPVGQTVPCHLRLSRPDTRASGRSADQAHSRRGGSAARPHQGQPLHPGGQRNRTRRTHALLAAGNVTATAADEGSAEWRWRWR

SEQ ID NO: 292 - protein, 197 aa, Mycobacterium tuberculosis
MTDNECPADSRRRHVLRLALFAGILLGLFYLVAVARVIHVDGVRSAIVVATGPIAPLAYVVVSAALGALFVPGPILAAGSGVLFGPLLDTFVTLPAFSAGAQAGMTPRRCWVSIAPIASMHRSNGADCGRWSVSASSPASRMRWPRTPSGRSEFRCGRWSLGRSSGRRHGCSSTPRWARRSPTCRRRWFTRRSRCGA

SEQ ID NO: 293 - protein, 144 aa, Mycobacterium tuberculosis
LWAVVGQRFVPGISDALASYTFGAFGVPLWQMVVGSFIGSAPRVFVYTALGASITNLSSPLVYSAIAVWCVTAIIGAFAARRWYRKWRARPRRRCGLAQLTTGSQQRHTSHRTPAGVVMPGSLSEHRRLRQEAPDRIEHHPPIE

SEQ ID NO: 294 - protein, 165 aa, Mycobacterium tuberculosis
LSAVLPARCIRALADRVYRHVRCHGGCARNHHPRSRPGRIDYLGVHRGQRVPGAKGWIDIRHFHTGRGDLDGRAAVVRQPLSGGEQYCSDDRVGGRHAVVDHLRVTGTAHDRLVERVSVLDNGGGVCTGRDPWRHVLNSVAPRTRHRIRPAVPRRRCRSRGSQDR

SEQ ID NO: 295 - protein, 67 aa, Mycobacterium tuberculosis
VGPMNGFLSWWDGVELWLSGLPFALQALAVMPVVLALAYFTAALLDALLGRVIQLIRRARRPDQAPR

SEQ ID NO: 296 - protein, 577 aa, Mycobacterium tuberculosis
MADDVSGAVYRAGTAHGRPTGRIEHRDRQVVTRRATDTRAELDGLSDHQLAEVQRSRENHYPAGCLVIPQPLNRRPEHQPAPPQRHWALAGGDRDQRGGAKCHGDWVAIDRLGAQRDRKPVPRAHHTDRDQAGADRTQSRSVPRPARHTPPQCAAAEGHHDAAQGTHVADRPHDPGRRHNPADQRRRDQAYVQTGRAEAHMAHRYQTRTRLRRLSSRAGPMPSTSASWSTLVNLPLRCRHATIAAAVTGPMPGRASSCSTVAVLRSSTSAVLGAVLGPVVAVPEVPAGPGVPAPTELPSTLGCPEGGASPTTICSPSPSCRAMFSPTVSAPSTAPPAACSASAIRAPGARVTSPGVCTRPTTLTTTGRPERSGEPGLADDLGFVGETGSTGGSLADITGSVRSRIKVNTVTSTARAAITANATAPARPGSARILSAQPCPREVSGSQRGSSEFGSSRGSSWSGPSSVGSCGSGSKCADAACESISGTAPSRLCSRSAGSSVRMGRPQLRGPPEPARTTASRCPAVDQSEAVDKPLWRWIKMGQTAPTSPNNQHRAATSIRTRLTAIESVLGNAIREC

SEQ ID NO: 297 - protein, 88 aa, Mycobacterium tuberculosis
MRGTAYATRRSMLPNTRAVWLATVVQCVTGGLGVTLIPQTAAAVETTRSRLELARFVAPARRDESVWCLALSAAARSPTSVLPGLSAS

SEQ ID NO: 298 - protein, 402 aa, Mycobacterium tuberculosis
MRRVFSGWTTLVRCSTAATTVTIRAMTKHVPVIHSSEPTRPLTPRKPVDPVRRCCSQLRQPNPTTLKTAKSASAKSAAVVVRISGSPSVRSSGHEPLWRLARRAASIAAAAGAQSPTPDTKVASAPAAQARRNARSRSAGEPGLRGQFAARTPANTSPAPAVSTGVTVGAGTSNTPSAPTYRAPRTPRVITKCVGGDGQSFASCSLAITTSAIAAKSCKELRSWPAGEALTMTTASADWAARAAASAVATGISNWVNSTSQLATAEGTGVRCALAPGATSTVFSALASTTIIAVPLGPGTVTVLSSPTALARRWARSWAAAGSSPNAPENCTCAPARAAATAWLAPFPPGVRVNDAASTVSPGRGSASTTNVRSMFTLPTTHTRGAMGPTLVSLAFAMLAVG

SEQ ID NO: 299 - protein, 226 aa, Mycobacterium tuberculosis
MSASASADKVVCECCELCVPKQLASAIRNPYGLVRGWRCRICNEHQGQPVKMAQDHEEEVRIRWGETVDELHAALDRAGPRPGTWCTSEGSSRDPSGGSLGGQCWGVGGLLLGGFFGAGQCCSGSGEDLEAQVAPSFDPFVVLFGEDGSDEADDRGAVGEDAHDVGSASYLSVEAFLGVVGPDLAPDLLGEGGERQQVGAGGVEVLGHRGEFVGQSVEYPIILGNN

SEQ ID NO: 300 - protein, 622 aa, Mycobacterium tuberculosis
VGSLTVFTSSARMSRTAANTSARALHSMTTGSGGKSRMLNTIASPPPTSASKRRSKTTLPLDAKTKTSTAETAAWLMNIKPCATNPRAHSTATDNAMNTTTPMAIGPEPSRACTPAPSTIPSATPTIICWARRARSTLVADMHTTAEIGAKNACGWLNTSWVRYHARPATTDVWVIGHSTVRNRWATARPPQAVLTSSEALMNAYLFTLGCDVVTAKIWSCLLDPLGLGIYSGLLTLLSGNGRRRVGERIDAAAGLRERDHLTDRVHPGQQRGGPVPPERDSAVRRCAKHERLQQESELFLRLGLVQAHHREHPFLDITAVDTHRAATDLVAVADDVVRVGQHAAGIGFDAVLPFRFRRGEGMVHRGPGPRADRDLTGGGRFVGRLEQRRVNDPDECPRIGVNQAQPVGDLDAGRAQQCPRRFDRTGREEDAIAGFGPDMVGQSGALGLGQVFGHRTAQRAVFGDQHVGQSAVAALLGPVLPAVQRAPRLRRPARHHHRAHIRCLEDTKCGVGEEIRAFDELQPEPQVGFVRTESAHRFGIADPRDGRRNPVAYQRPQLGQNFLGDRDDVLGVDEAHLHIELGEFGLAVGAEVLVAVAAGDLVVAFHPRHHQQLLEQLRALR

SEQ ID NO: 301 - protein, 328 aa, Mycobacterium tuberculosis
VIGDFAEMLGGQDGVAELVQHVAVHPFDGVDELVEADGVGGGCGLRHDVNSRLTLCIVSTVIGCVVGSAALPGRCGQGGADRGHQAGVGVAGDQRDPGQAAGDQVAEERQPAGPVLGGGDLDAQDLSVALGVDAGGDQGVHPDDAACLAHLEHQGVGGEEGIRAGIERAGPKRLYGFVELFGHDRHLRLGKLCHTKCFDQALHPASGYSQQVAGRHHAGQCAFSSLAALQQPVREIAALAQLGDRDVDGCGTGVEITVAVAVALIGPLIAAFAVARPAQGVGFSPHQGGDERREQPAQQIRARLCELVSQKLLGVDKMRRGHCVISFD

SEQ ID NO: 302 - protein, 126 aa, Mycobacterium tuberculosis
LDEPAHRARPKGNGANHDGAQPCCGIGACGNRGDPRARAHLPLPKGGRAGGAWHGVHRRPRRNLRASRSQRRGQVHHPEASHRAAARPRRPGHGVGQRAGRVGTRLLRAHRGLLRAAQPLPKAHRV

SEQ ID NO: 303 - protein, 178 aa, Mycobacterium tuberculosis
MIPQMTVSCPPPSTSEREEQARALCLRLLTARSRTRAELAGQLAKRGYPEDIGNRVLDRLAAVGLVDDTDFAEQWVQSRRANAAKSKRALAAELHAKGVDDDVITTVLGGIDAGAERGRAEKLVRARLRREVLIDDGTDEARVSRRLVAMLARRGYGQTLACEVVIAELAAERERRRV

SEQ ID NO: 304 - protein, 484 aa, Mycobacterium tuberculosis
LVTTLAPILDSASMTPKTASSLPGISDDDNTMRSPAVNVMLRCSPRDIRDNADIGSPWVPVVINTTWSGAIVSAAAMSMRSESATRRKPSCLATRMLRTIDRPTNDTRRPNATAASMICCTRSTLEAKHATITRPSAPRMSRCSVGPTSLSDGPTPGISAFVESHSNRSTPVSPSRDMPGRSVGRPSGGNWSNLMSPVCRMVPAPVYTAMANASGVEWLTAKYSHSNTPCRVLWPSRTSTNTGVMRYSRHFSATRAKVNFEPTTGMSGRSLSRNGIAPMWSSCPWVNTSASMSSSRSSTWRMSGRIRSTPGSSWPGNNTPQSIINSRPRCSKTVMLRPISLMPPSAVTRNPPEVRGPGGGRSTSTSGPPFGSPLDHRSTEAARMSAANASICSGVAATWGSRGSPTSMPCSRKPALDNVTPPRRLIALHSGATAMLILRAVAISPEPKADNNSRSCPAARWAITLMKPVAPMASQGRLSASSPE

SEQ ID NO: 305 - protein, 86 aa, Mycobacterium tuberculosis
MSKRSDGPSTGNAIRARHRISVMTAQRSTSHATRTPVASSAQLGPPSSVEPTVRPGLAGLVAVKRGREAAARLPNNPETGCKSRDH

SEQ ID NO: 306 - protein, 158 aa, Mycobacterium tuberculosis
VATKNAAWPSSTSCSNYSPNATIESQRPDGCTSSRACVTPPVTQRLFSSLLTGYTNGSKIRQTPSNSRPRCTSTSIALARRSPNERHPRRLCETGRSNSRPAKEKERLRADHNPAAGATQPDRTALRRGAAERQPHAPASAEGEGPVPAGPVRLPVRA

SEQ ID NO: 307 - protein, 93 aa, Mycobacterium tuberculosis
MSAPDVRLTAWVHGWVQGVGFRWWTRCRALELGLTGYAANHADGRVLVVAQGPRAACQKLLQLLQGDTTPGRVAKVVADWSQSTEQITGFSER

SEQ ID NO: 308 - protein, 178 aa, Mycobacterium tuberculosis
MLHDVVHGRRCSENGHRRRITQYRIGTFIGNAALWNRKRHGDAPGLQRAEKGDDVLESLRSRDHHAVTRGTTTAQLLCHIQRSPIQLRPRQGYRNAVPVLFVIHKREGRVMGLQTRTRAQRSGKGTHTHGHHVTGHAWSCRSRRRGVLALRGLSQVASGQLSRGLPARHGSTIGHGRM

SEQ ID NO: 309 - protein, 176 aa, Mycobacterium tuberculosis
MPTTKATQRRDVSTEIAYLTRALKAPTLRESVSRLADRARAENWSHEEYLAACLQREVSARESHGGEGRIRAARFPARKSLEEFDFEHARGLKRDTIAHLGTLDFITARDNVVFLGPAWHREDSSCGRPGDTRVSGRSSGAVRHRRRMGSTARRGSPRRAHLRRTHPALPLSAPGG

SEQ ID NO: 310 - protein, 156 aa, Mycobacterium tuberculosis
MQWGYRPLAGDEAMRWGYRPLARESGALDPDHRRCRRRPAHCRPTTRNQTYHRSGARVAIQHRDCAAGSDRSGGVGPLCGFRRPGAGGVVAGSGVRAVRGVRPAQRGRHCAQHRGPRSLRCDAAPGRGGGRRGGRDHVPGGSGVGRPALQRRLRRR

SEQ ID NO: 311 - protein, 281 aa, Mycobacterium tuberculosis
LGGVASTRQASVRRWSAVHPLDASPALPRPGQRCATARAVAGPTPSWRAAVRSAGVSTSQRRPGQAPVSSTAPERRCRADESGPNRGCSAVPNAHSTAVPVPSRSATKLRRWWRAAEIASASSCVCNAGKSPCSTTMLEAPSATTRSAAVMAVFSGSGSSSGVGSASTSAPSPAAAAAAASSGVITVIERSEPTPAAAVNVSTSMASTTFSRVCAENTGASLVLAAAKRLTAMIKPISPSSGVPLMKSSCQRRSTRHTSTALPPRSWPGPRHGPDGNRGAD

SEQ ID NO: 312 - protein, 278 aa, Mycobacterium tuberculosis
LRGRLIRYAVLLSPSLPLRPSASATGFQSASVVVTAERALPAWPLPAPPLEPELHAASITAAAVVIATILPACLAPAMRVPSIRCIHGVDGSSVSHGLSGDYETTMKLDRTDPGTARRPHRRPGRVSAGRRGSSTRGTHAHPRRGHQRHRPTCPSAIATGSRRNPVSWNNIQRPSAAAARRARARTSIRQRCGPRTSHPLSLLTTELELALRRPRSNPELLAAIRSALAETTDTARTTGGTGLGLAIVDTLSQRNHASVTARNRAAGGAEISLRLALG

SEQ ID NO: 313 - protein, 185 aa, Mycobacterium tuberculosis
LLGLPDPRPVPRNPAARRRATSRSLSADPSSRPASQSRPRPGTWCTSEGSSRDPSGGSLGGQCWGVGGLLLGGFFGAGQCCSGSGEDLEAQVAPSFDPFVVLFGEDGSDEADDRGAVGEDAHDVGSASYLSVEAFLGVVGPDLAPDLLGEGGERQQVGAGGVEVLGHRGEFVGQSVEYPIILGNN

SEQ ID NO: 314 - protein, 310 aa, Mycobacterium tuberculosis
MIFWATRYCTIWLPPSPSSVTFSPAVLAGLGVDASTVDPALASPTSSLSTPISARVSTVTSFFLAAMMPLKDGKRGSLIFSFTLITAGSVASRVNTPSSVSRSPVILPPSIDTLRRWVSCGRPRYSAMMAGTAPPTPSVDSLPAITSSVPSMVPNARAKAHPVWMTSEPCMPSSFRWTALSAPIDSALRIASVARSGPAVSTVTDPSMPSAASFSRICSASSTARSLISSSTASAASRSSVKSPSVSLRSDQVSGTCLIRTTMFVMTVVRPPRRRPAALDCGTSVTRFATAQRYYYSVSSRGAPSHHSGW

SEQ ID NO: 315 - protein, 152 aa, Mycobacterium tuberculosis
LLSSWPRPGTWCTSEGSSRDPSGGSLGGQCWGVGGLLLGGFFGAGQCCSGSGEDLEAQVAPSFDPFVVLFGEDGSDEADDRGAVGEDAHDVGSASYLSVEAFLGVVGPDLAPDLLGEGGERQQVGAGGVEVLGHRGEFVGQSVEYPIILGNN

SEQ ID NO: 316 - protein, 215 aa, Mycobacterium tuberculosis
LIRSIDRWGSAAGGAVGTPGGTDCNGRSSHPARSAATNTSISAQGAAGPWVKNRGRSSFPVASCSRTAAETTSSCLGSGAPATNVSARQPDTTYRPSVDRTGRARRTPSTNNVSRTRADQAARALSATIDNTTSPHRQPPSQPAPNRMGCAPAKPNATNTCSGGGSTFTPVSLVEPIGVYWACIGPSTSPCRAASAWPTRRSHPAGVPRRRNRLS

SEQ ID NO: 317 - protein, 298 aa, Mycobacterium tuberculosis
MRCRAALSWRLPERLSRIWPAVLPDHTGMGATAAWQAKAASLLNRVTPAASPTILAAVSSAQPGISSSAGATWWTRALMRWARVLISPVSRMMSVSSARASSATNPGWVSSQVRRACWCLAASSERAAGARSGSSSWTSQRNRLIADVRWATRTSRRSVNNFNSRDVSSWVARGRSVSRSTARATASASIGSDLPRLRADLRVWAISLVGTRTTCWPAASRSRSRRADMLRQSSMPQISSRPNCSRAHMMAVACPAVVALTVFSPSWRPTSSVATKVWLYLCASVPTTTMVVASEPPR

SEQ ID NO: 318 - protein, 515 aa, Mycobacterium tuberculosis
VPDLLEFAALGLRRKAVHDHERDDGERAEDRENACAAEHTQHGEREGGNDRVGRQCRGEHRARSHRPQPGREALRGVHPDQRAESEVEPDDEQQHAGEPQHQPRATIGVVGEYGDQHGICGDHRRDAGQQDRATAQPIDQKQRGTHRRQAGDLHHRGQGKHREIAREAHGGEKSRTVVDDRVDPGDLDEEAERDDEQRGPQIRPPHHFADTAAAFVDRGRHIGQLGIDVGLRLDPPQRATRVGDPALEQIPAGGIGHAPQQRQQQRGRRGGQPEHRAPAVRSGQQVADQVTDDDAAKRRQLIRGHQRPTHRRRRRLGHIHRHHHHRQADCHTQQQTRHHQHRYGHRGRAEQGEHCVAGDDEHHRFLASDRVGEDAAAKRPGDLAEHRRGGQQLLFSSGEFEFLAERQQRTRDGGKVVPVEDADAGGGEPDEERPAPRSGQLTGTGALSTSTTRSGSSGAPAGVNPASWYRAVVISMRLPQRRHAVNRWSSPDFGADQGRLGCPPANDAEGIGVSS

SEQ ID NO: 319 - protein, 376 aa, Mycobacterium tuberculosis
VSDATTVLFGLPGARVERVERRSDGTRVVDVITDEPTAAACPSCGGGLDISEGIRGYLTERSTLWRRPHHGALEQNSLAMPRRLLQAGAVHRGHHPGTCPRPQHAAAASADGQGDRGCGPLGGPRSPRLTPCRGRRHIGRLLPTPRRVLTEPLPTPVLGVDQTRRGKPRWERCAKTGRWVRVDPWDTGFVDLAGDQGFMGQHEGRGGAAVLAWLQARTPQFRESIQYGGHRPRRCLRLGDPHARAAAQRQARRRPLPCDHAGQRRADRGAPPGDLGVPRPARPQDRPAVGQPTSLADRPGTLVGQKLRQNAESDQRRRPPRADSLGLDRQRGAAHPAVDRAHRRGPPPGAPSPTPLPAWRIDSQIPELLTLATTID

SEQ ID NO: 320 - protein, 72 aa, Mycobacterium tuberculosis
VQALPESQLPELAVQMRRRLIETVTATGGHLGAGLGMVELTIALHRVFTSPHDIGVRHRAPNLSAQAAHRPR

SEQ ID NO: 321 - protein, 239 aa, Mycobacterium tuberculosis
MSSEGGWPNVGNLARSASMTSAVSSSARVVWVRYDSWVPSGRFNAATSAGVCTNNVDRGATPSVPSVSSCPACPMKTTVSPRAANRAASAWTFATSGQVASTTCSPRSSAPARTAGETPCAENTTTAPGGGGSGISSRSSTNTAPRSRNSATTTVLCTICLRTYTGPSATSSTRLTVSIARSTPAQNDRGDANSTVTSPEAYPCATGPTNTSAISTPGDISVATTRSGLGIAPHRAVPQ

SEQ ID NO: 322 - protein, 173 aa, Mycobacterium tuberculosis
LPGHRRGTSASRVPGNRPRLRPSWPRRTPLARPKTTGCARSTCSSRARARAARPRSGRCRPPAWRWARSRMSPPSRITVSGPPSAGASRREDGSLHRTRHPQITAVAHRPRRWRPGLREASLPARPTRSRADQGKRISASAAGEAEGPFHIRRNGKAVPPLLRRGRAAARQDG

SEQ ID NO: 323 - protein, 213 aa, Mycobacterium tuberculosis
VGRRDRGAPARPFSAHPQRRCLLAGQSQGCRRGIGLRPARQHLVGGGSGGPGGAGELRRRQGWHHRANPVGGAGARALRRLRQCDLSAGAHRDDGRCLRRRTRCRSGPDRPAVAAACGKPGPVSGVPGCRGSQRSGVHRLRSAGDAGVTAAHGAPVQRGRHVLGSHRAHRDAAGLLCWSGSGTELFGDRSDASVTRGYRRPIIGIGVRITTPT

SEQ ID NO: 324 - protein, 191 aa, Mycobacterium tuberculosis
LPWTACCSPYSNDNRTKPSPVKSATNSSPARASTANVHDPGNTMSPLRSRTPKLSTLPASQATAVAGLPNTASLRPSATTSPLRVSFASIALTSRSAGGTRAAPNTKPAAEALSAMVSQILICQSLILVSISSMAGTRASVAASTSSSVQPAPGRSAARMKPTSTSTRGDRYRDAWTGVSSNTCMSSSRCP

SEQ ID NO: 325 - protein, 212 aa, Mycobacterium tuberculosis
VVHSRRSWAPSRRPHRGIDAANERAPAVPEQLTGDPDDRPAQIQQRGGPLDVPSPLRRVCPMLWPVILDTDSQLLVAQVDAGDEVPVVVKHSDLCLRLRQTGIDQHQSGPRLLWGFRTPVDQRQHRTEADQAARTGMFGNDGLHVGDLDIGRIRQRVQPLNGLQPRGCAPPDIEGGARRGGYRDTVNRNRLVRRQSIRVHDDARRRLSIGVH

SEQ ID NO: 326 - protein, 234 aa, Mycobacterium tuberculosis
VSRYPNSWRRLNNPDMAVPMLNRPVFKPLRTEPKRVPGTPMLPMPEVWPLMTVPPLAVLKNPETSTAKGPVGVLKKPETSVPVLPKPELVRPLSVMIPKPVFTLPAFHEPVLMLPEFPLPVLTLPELSNPVLTKPAFPKPVFNSPAFPKPVLRMLAFPKPVLRTPAFPKPMLALPEFPTPRLLRSPGTRVLAPVLKTPMLPLPELNKPMLLVPELPMPILPLPEFSSPARLMPI

SEQ ID NO: 327 - protein, 80 aa, Mycobacterium tuberculosis
LSSNHAILRLLAPLRLDPQNLGAGPQREHRHRQGRRHGAQSQSGVLADAGVDVVPAQHAPPQQVRQRTGIGQVGSDVDPE

SEQ ID NO: 328 - protein, 114 aa, Mycobacterium tuberculosis
LCQGVPARLPPATDTVGVVTKSAVPRVGLDVQIDYSLGDRPVPGHGTGTNQETCEAVCYGAVRRFASGQAQGGDHLGWPGRHRARGRAAARRPCCGGVQRHLSCVPAARAAPAA

SEQ ID NO: 329 - protein, 200 aa, Mycobacterium tuberculosis
MRPAKRAEEEPGNHPRAGCSGSPPSAPWRSQTPRLATMRPAKRAEEEPGNHPRAGCSGSPPSAPWRSQTPRLATMRPAKRAEEEPGNHPRAGCSGSPPSAPWRSQTPRLATMRPAKRAEEEPGNHPRAGCSGSPPSAPWRSQTPRLATMRPAKRAEEEPGNHPRAGCSGSPLARPTTGSSRRRRKIRQLSVRVKHAVHRT

SEQ ID NO: 330 - protein, 74 aa, Mycobacterium tuberculosis
MRTTIDLDDDILRALKRRQREERKTLGQLASELLAQALAAEPPPNVDIRWSTADLRPRVDLDDKDAVWAILDRG

SEQ ID NO: 331 - protein, 118 aa, Mycobacterium tuberculosis
VSRCRIHCRRLALSRQKTRSLPDLQLASRSGLRRLGCKMDVIRWARRLAVVAGTAAAVTTPGLLSAHVPMVSAEPCPDVEVVFARGTGEPPGIGSVGGLFVDALRFPGWRQVTRGLRR

SEQ ID NO: 332 - protein, 137 aa, Mycobacterium tuberculosis
VDACHSRARRGVVDRRRPRCGGTARGVVGIRAWAAPLHCGRSSDSGARARENSGRVAGTTMLAVPVPDSALRVAGSVLDQAGPYLPFNTPFTAAGMQYYTQMPESDDSPSEKELGITYRDPRDTVADTVTALRGLGS

SEQ ID NO: 333 - protein, 243 aa, Mycobacterium tuberculosis
LMWKPRWRWCSTASERRTTASPDACRNVSRCRSPSLRLAGSGSPWHRMRSRSTAAMATSRPGRWPGCCVTRKSTRSGRAPTTSCVWMCGAGSSRRALTRHCWRGCAMRCRCPTMTTPRGWSRAALRTSTRRSPLGPNSTGSWPRRGCSRWPNSWATSTPARCSPSRPPGNGQPAAPTARHSSPACTRAGISPTKARCAVSTQIAMRRCSVSTNSWRARSLPSRRKSPQFVALLTLPWVSLCPE

SEQ ID NO: 334 - protein, 197 aa, Mycobacterium tuberculosis
VRAPATRAASRGSSRNSDQRPSGRSVIPSRPSSSACQVCSGVFISPGKRVDKPTIAMSTRSAGPVRDQSSASSPARSVSGSPSMIRVASDSMVGCANATATDSVTPVRSSMSAAIATASRDDRPSSTMGTDSSIESGAFPTALPTQLRSHWRISGTVSSALSAGAFSWDSATSAMGPQSEVAKTVGEPTPLRRLPSR

SEQ ID NO: 335 - protein, 230 aa, Sars coronavirus
LHEDPHTGVEPGAVTAHRDCQHPRPACGDEPFNPACVLVRTDGPDDRKCEMTAIRFDAHRSGRECHAVLIAAFLLEPGEAHCLALTFTGSGVLPVPVRIDSAANAVGVSLFRALRPPHRPGLGVDTHLVLDGVPPFTKHPQRRLDSPDTSNAPRLDIGFQSSDRPVVGLAASAEMPRQRAGLVLGWVQREPERLHTPAFWHLESGHQAASASPTAAARARLAPFCAARSP

SEQ ID NO: 336 - protein, 148 aa, Sars coronavirus
LRPSRSTLIAKCASWRQPPRCLRSAAVNRRSSAPVAQRELRAENRPESRPQFTLGAVWPHPVNVICAGGRWRVANPSGAGPPSTPRRGQLISGYASATAPAMGCGRTRRISPNTRMPSCRAHLLKEGLRHLFSVKGEESKQALDRLIF

SEQ ID NO: 337 - protein, 178 aa, Sars coronavirus
VHSASSVATPVRGSTLAGSAGPSTAVTMPAKPTCGATNCSTSMSPSRAAITWRSPLRHTTKRTMTPPMSRRHQRPSKVRSGLPRVSTISATVGWGSPWRSSTPCAVRSRCTCSQTMSRRSSCGIFGRIPSVTGKSTRCNRSAITNMPSMVTSTPTTLSAVPARPAADGPVMINRKSCR

SEQ ID NO: 338 - protein, 153 aa, Sars coronavirus
MDRLCGAPLCHRRRGPTATAAQAGARRLHDPQQAPGRAVAGQLRPAGRADRGAGRPGGSGSGAPRPGRQPDHGGARHSGGPASRRGVALLEGAAARARPVVHRGGDNRAAVLVEITGEPLAWESRQNGCGVLHSRRRRQRRDLEPPVRRRPRR

SEQ ID NO: 339 - protein, 8 aa, SARS Coronavirus
RIRASLPT

SEQ ID NO: 340 - protein, 8 aa, SARS Coronavirus
RSETLLPL

SEQ ID NO: 341 - protein, 8 aa, SARS Coronavirus
LDKLKSLL

SEQ ID NO: 342 - protein, 8 aa, SARS Coronavirus
ATVVIGTS

SEQ ID NO: 343 - protein, 8 aa, SARS Coronavirus
NVAITRAK

SEQ ID NO: 344 - protein, 9 aa, SARS Coronavirus
LQGPPGTGK

SEQ ID NO: 345 - protein, 8 aa, SARS Coronavirus
RIRASLPT

SEQ ID NO: 346 - protein, 8 aa, SARS Coronavirus
RSETLLPL

SEQ ID NO: 347 - protein, 8 aa, SARS Coronavirus
LDKLKSLL

SEQ ID NO: 348 - protein, 8 aa, SARS Coronavirus
ATVVIGTS

SEQ ID NO: 349 - protein, 8 aa, SARS Coronavirus
NVAITRAK

SEQ ID NO: 350 - protein, 9 aa, SARS Coronavirus
LQGPPGTGK

SEQ ID NO: 351 - protein, 8 aa, SARS Coronavirus
TLSKGNAQ

SEQ ID NO: 352 - protein, 8 aa, SARS Coronavirus
VAQMGTLL

SEQ ID NO: 353 - protein, 8 aa, SARS Coronavirus
LVLVLILA

SEQ ID NO: 354 - protein, 8 aa, SARS Coronavirus
TQTLKLDS

SEQ ID NO: 355 - protein, 7 aa, SARS Coronavirus
GLLHRGT

SEQ ID NO: 356 - protein, 8 aa, SARS Coronavirus
LLPLLAFL

SEQ ID NO: 357 - protein, 8 aa, SARS Coronavirus
LLLFVTIY

SEQ ID NO: 358 - protein, 8 aa, SARS Coronavirus
QTLVLKML

SEQ ID NO: 359 - protein, 8 aa, SARS Coronavirus
DDEELMEL

SEQ ID NO: 360 - protein, 8 aa, SARS Coronavirus
LIVAALVF

SEQ ID NO: 361 - protein, 8 aa, SARS Coronavirus
RARSVSPK

SEQ ID NO: 362 - protein, 7 aa, SARS Coronavirus
QLLAAVG

SEQ ID NO: 363 - DNA, 30 bp, Artificial Sequence
  Illustrative polynucleotide sequence
atgcctaagtaccgttccgccaccaccact

SEQ ID NO: 364 - DNA, 30 bp, Artificial Sequence
  Illustrative polynucleotide sequence
caccggaatgaccgacgccgatttcggtaa

SEQ ID NO: 365 - protein, 10 aa, Artificial Sequence
  Illustrative peptide
MPKYRSATTT

SEQ ID NO: 366 - protein, 9 aa, Artificial Sequence
  Illustrative peptide
HRNDRRRFR

SEQ ID NO: 367 - protein, 10 aa, Artificial Sequence
  Illustrative peptide
CLSTVPPPPL

SEQ ID NO: 368 - protein, 9 aa, Artificial Sequence
  Illustrative peptide
TGMTDADFG

SEQ ID NO: 369 - protein, 7 aa, Artificial Sequence
  Illustrative peptide
VPFRHHH

SEQ ID NO: 370 - protein, 6 aa, Artificial Sequence
  Illustrative peptide
PTPISV

SEQ ID NO: 371 - protein, 33 aa, Artificial Sequence
  Illustrative peptide
AGTFYRYMGHVNMKIYTASLPTYRYGYFSHRED

SEQ ID NO: 372 - protein, 9 aa, Artificial Sequence
  Illustrative peptide
HGIEKSDWE

SEQ ID NO: 373 - protein, 6 aa, Artificial Sequence
  Illustrative peptide
DFGTRE